(19) United States
(12) Patent Application Publication
(10) Pub. No.: US 2026/0021107 A1
(43) Pub. Date: Jan. 22, 2026

(54) STIMULATOR OF INTERFERON GENES AGONISTS

(71) Applicant: INIMMUNE CORP., Missoula, MT (US)

(72) Inventors: Omer RASHEED, Missoula, MT (US); Uddav PANDEY, Missoula, MT (US)

(21) Appl. No.: 18/994,389

(22) PCT Filed: Jul. 25, 2023
(86) PCT No.: PCT/US2023/028578

§ 371 Date: Jan. 14, 2025

Related U.S. Application Data
(60) Provisional application No. 63/391,971, filed on Jul. 25, 2022.

(51) Int. Cl. A61K 31/5415; A61K 45/06; C07D 279/16; C07D 417/12

(52) U.S. Cl. CPC A61K 31/5415; A61K 45/06; C07D 279/16; C07D 417/12

(57) ABSTRACT

Stimulator of Interferon Genes (STING) modulators and their use for treating a disease, disorder, or condition associated with STING.

### BACKGROUND

Stimulator of interferon genes (STING), also known as transmembrane protein 173 (TMEM173) and MPYS/MITA/ERIS, is an endoplasmic reticulum-associated protein that binds to cyclic dinucleotides or cytosolic DNA to trigger cytokine production. STING is an innate immune adaptor protein that enables signaling from cytoplasmic receptors to the transcription factor interferon regulatory factor 3. Initiation of these pathways leads to the expression of type I interferons and proteins associated with antiviral and antitumor immunity. Compounds capable of triggering STING-dependent cellular processes are effective at blocking virus replication, enhancing vaccine efficacy, and facilitating an immune response to cancer cells.

### SUMMARY

In some aspects, the presently disclosed subject matter provides a compound of formula (I):

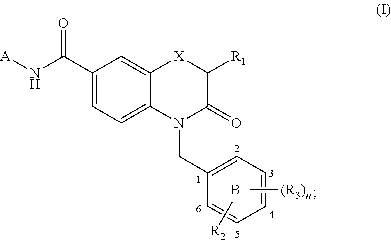

wherein: n is 1 or 2; X is selected from S, O, NRₓ, and CRₓ₁Rₓ₂, wherein Rₓ, Rₓ₁, and Rₓ₂ are each independently selected from H and C₁-C₄ alkyl; A is:

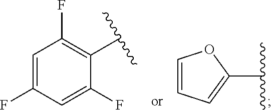 [SMILES: *c1c(F)cc(F)cc1F ; *c1ccco1]

R₁ is H or C₁-C₄ alkyl; R₂ is selected from —OR₄, —NR₅R₆, and —NO₂, wherein R₄ is H or C₁-C₂₀ alkyl and R₅ and R₆ are each independently H or C₁-C₄ alkyl; each R₃ can be the same or different and are each halogen; wherein: (i) if X is S, A is 2,4,6-trifluorophenyl, n is 2, and R₃ is F in the 2 and 6 position of ring B, then R₄ cannot be H or C₁ alkyl; or (ii) if X is S, A is 2,4,6-trifluorophenyl, n is 2, and R₃ is F in the 2 position of ring B, then R₄ cannot be C₁ alkyl; and pharmaceutically acceptable salts thereof.

In particular aspects, the compound of formula (I) is a compound of formula (I′):

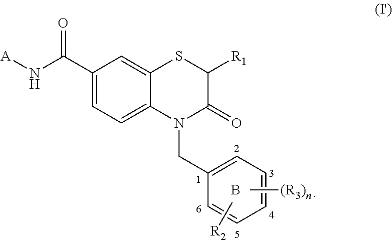

In certain aspects, the compound of formula (I′) is a compound of formula (Ia):

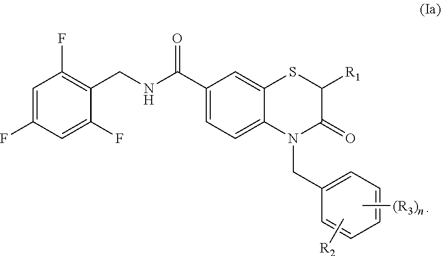

In certain aspects, the compound of formula (Ia) is a compound of formula (Ia-i):

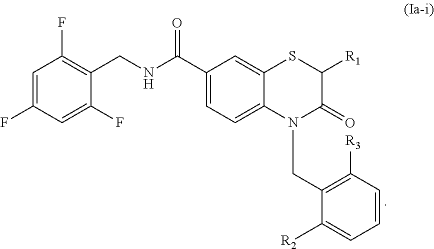

In particular aspects of a compound of formula (Ia-i), R₂ is —OR₄. In particular aspects, R₁ is H. In other aspects, R₁ is C₁-C₄ alkyl.

In certain aspects, the compound of formula (Ia) is a compound of formula (Ia-ii):

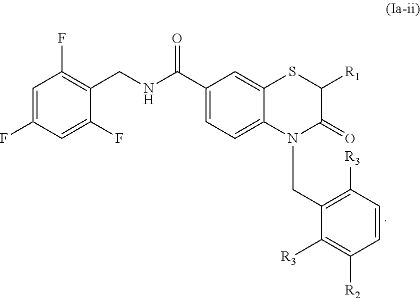

In particular aspects of the compound of formula (Ia-ii), R₂ is —OR₄. In particular aspects, R₁ is H. In other aspects, R₁ is C₁-C₄ alkyl.

In certain aspects, the compound of formula (Ia) is a compound of formula (Ia-iii):

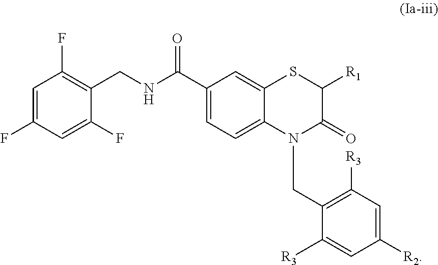

In particular aspects of a compound of formula (Ia-iii), R₂ is —OR₄. In certain aspects, R₁ is H. In other aspects, R₁ is C₁-C₄ alkyl.

In certain aspects of the compound of formula (I), R₂ is —NR₅R₆ or —NO₂. In certain aspects of the compound of formula (I-a), R₂ is —NR₅R₆ or —NO₂.

In certain aspects, the compound of formula (I) is a compound of formula (Ib):

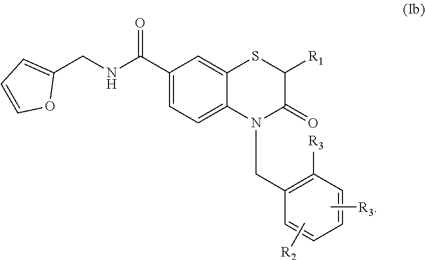

In particular aspects, the compound of formula (Ib) is a compound of formula (Ib-i):

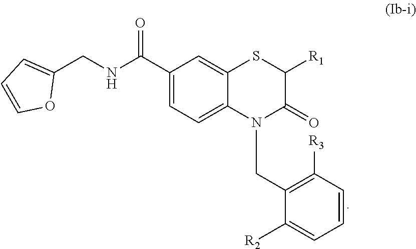

In certain aspects of the compound of formula (Ib-i), R₂ is —OR₄. In certain aspects of the compound of formula (Ib-i), R₁ is H. In other aspects of the compound of formula (Ib-i), R₁ is C₁-C₄ alkyl.

In certain aspects, the compound of formula (Ib) is a compound of formula (Ib-ii):

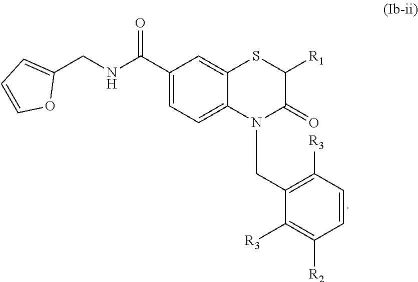

In certain aspects of the compound of formula (Ib-ii), R₂ is —OR₄. In certain aspects of the compound of formula (Ib-ii), R₁ is H. In other aspects of the compound of formula (Ib-ii), R₁ is C₁-C₄ alkyl.

In certain aspects, the compound of formula (Ib) is a compound of formula (Ia-iii):

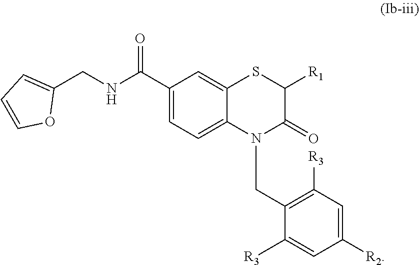

In certain aspects of the compound of formula (Ib-iii), R₂ is —OR₄. In certain aspects of the compound of formula (Ib-iii), R₁ is H. In other aspects of the compound of formula (Ib-iii), R₁ is C₁-C₄ alkyl. In certain aspects of the compound of formula (I-b), R₂ is —NR₅R₆ or —NO₂. In certain aspects of the compound of formula (I-b), R₂ is —NR₅R₆ or —NO₂.

In other aspects, the presently disclosed subject matter provides a method for modulating Stimulator of Interferon Genes (STING), the method comprising contacting a cell with a compound of formula (I), formula (I-a), formula (I-ai-iii), formula (I-b) and formula (I-bi-iii).

In other aspects, the presently disclosed subject matter provides a method for activating or agonizing STING, the method comprising contacting a cell with a compound of formula (I), formula (I-a), formula (I-ai-iii), formula (I-b) and formula (I-bi-iii).

In other aspects, the presently disclosed subject matter provides a method for treating a disease, disorder, or condition associated with STING, the method comprising administering a therapeutically effective amount of a compound of formula (I), formula (I-a), formula (I-ai-iii), formula (I-b) and formula (I-bi-iii) to a subject in need of treatment thereof.

In certain aspects, the disease, disorder, or condition is selected from cancer, a bacterial infection, a viral infection, a fungal infection, a parasitic infection, an immune-mediated disorder, a central nervous system disease, a peripheral nervous system disease, a neurodegenerative disease, a mood disorder, a sleep disorder, a cerebrovascular disease, a peripheral artery disease, and a cardiovascular disease.

In particular aspects, the disease, disorder, or condition is cancer. In more particular aspects, the cancer is selected from colorectal cancer, aero-digestive squamous cancer, lung cancer, brain cancer, liver cancer, stomach cancer, sarcoma, leukemia, lymphoma, multiple myeloma, ovarian cancer, uterine cancer, breast cancer, melanoma, prostate cancer, bladder cancer, pancreatic carcinoma, and renal carcinoma.

In some aspects, the presently disclosed method further comprises administering a second therapeutic agent. In certain aspects, the second therapeutic agent is selected from an antiviral agent, an anti-inflammation agent, a chemotherapeutic agent, an anti-cancer vaccine, and hormonal therapy.

In particular aspects, the second therapeutic agent is selected from a B7 costimulatory molecule, interleukin-2, interferon-g, GM-CSF, a CTLA-4 antagonist, an IDO inhibitor or IDO/TDO inhibitor, a PD-1 inhibitor, a PD-L1 inhibitor, an OX-40 ligand, a LAG3 inhibitor, a CD40 ligand, a 41BB/CD137 ligand, a CD27 ligand, Bacille Calmette-Guerin (BCG), liposomes, alum, Freund's complete or incomplete adjuvant, a TLR agonist, and a detoxified endotoxin.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 shows the THF luciferase activity for compounds INI-3067, INI-3069, INI-3070, and INI-3071;

FIG. 2 shows the luciferase activity of THE STING KO cells for compounds INI-3067, INI-3069, INI-3070, and INI-3071;

FIG. 3 shows the luciferase activity of THE TRIF/MAVS KO cells for compounds INI-3067, INI-3069, INI-3070, and INI-3071;

FIG. 4 shows the THF luciferase activity of compounds INI-3077, INI-3078, INI-3079, INI-3080, INI-3067, and INI-3069;

FIG. 5 shows the THF STING KO luciferase activity of compounds INI-3077, INI-3078, INI-3079, and INI-3080;

FIG. 6 shows the THF STING only luciferase activity of compounds INI-3077, INI-3078, INI-3079, INI-3080, INI-3081, and INI-3067;

FIG. 7 shows the THF luciferase activity of compounds INI-3067, INI-3111, INI-3112, INI-3069, and INI-3110;

FIG. 8 shows the THF STING only luciferase activity of compounds INI-3067, INI-3110, INI-3111, INI-3112, and INI-3069;

FIG. 9 shows the THF STING KO luciferase activity of compounds INI-3110, INI-3111, INI-3112 and IFNβ-plate 3;

FIG. 10 shows the THF luciferase activity of compounds INI-3071, INI-3105, INI-3106, INI-3107, INI-3108, and INI-3109;

FIG. 11 shows the THF STING KO luciferase activity of compounds INI-3105, INI-3106, INI-3107, INI-32018, INI-3109, and IFNβ-plate1;

FIG. 12 shows the THF STING only luciferase activity of compounds INI-3071, INI-3105, INI-3106, INI-3107, INI-3108, INI-3109, and INI-3069;

FIG. 13 shows the THF luciferase activity of compounds INI-3070, INI-3074, INI-3071, and INI-3075;

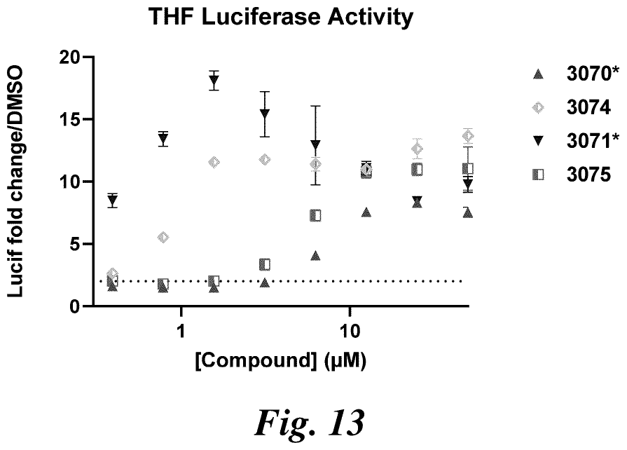

FIG. 14 shows the THF STING KO luciferase activity of compounds INI-3074, INI-3075, and IFNβ;

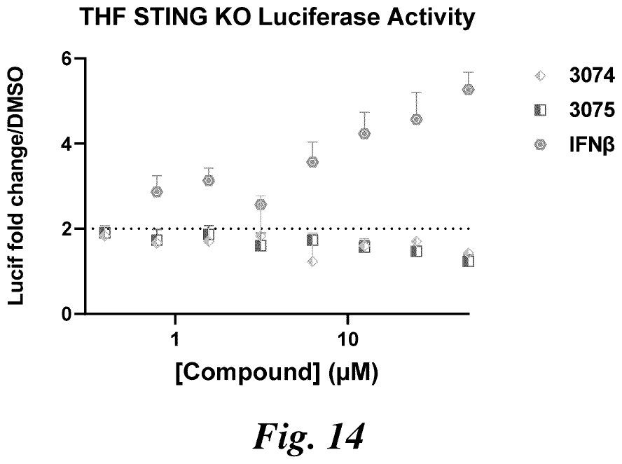

FIG. 15 shows the THF STING only luciferase activity for compounds INI-3070, INI-3074, INI-3071, and INI-3075;

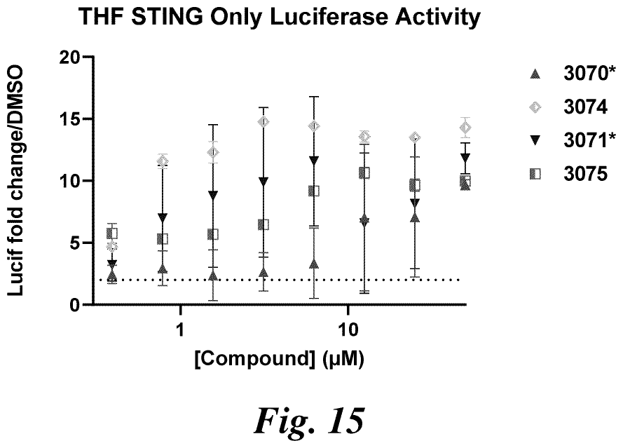

FIG. 16 shows the THF luciferase activity of compounds INI-3076, INI-3082, INI-3083, INI-3084, INI-3085, INI-3086, and INI-3069;

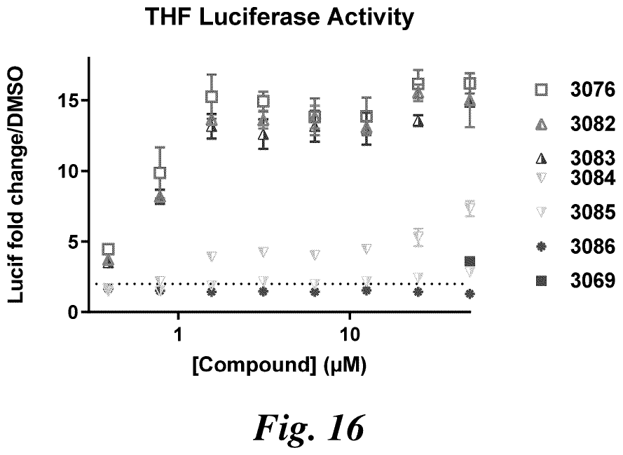

FIG. 17 shows the THF STING KO luciferase activity for compounds INI-3076, INI-3082, INI-3083, INI-3084, INI-3085, and IFNβ;

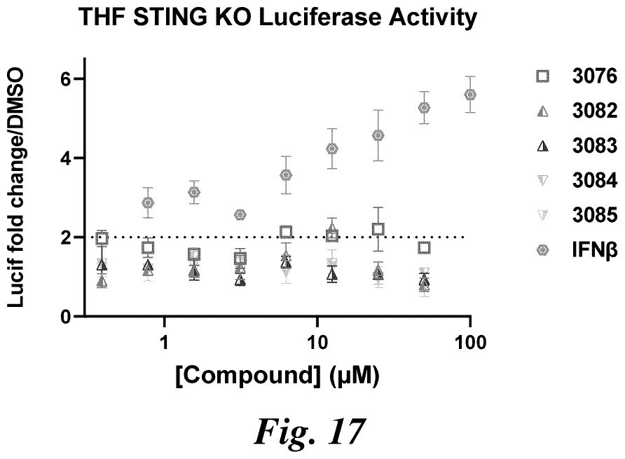

FIG. 18 shows the THF STING only luciferase activity for compounds INI-3076, INI-3082, INI-3083, INI-3084, INI-3085, and INI-3086;

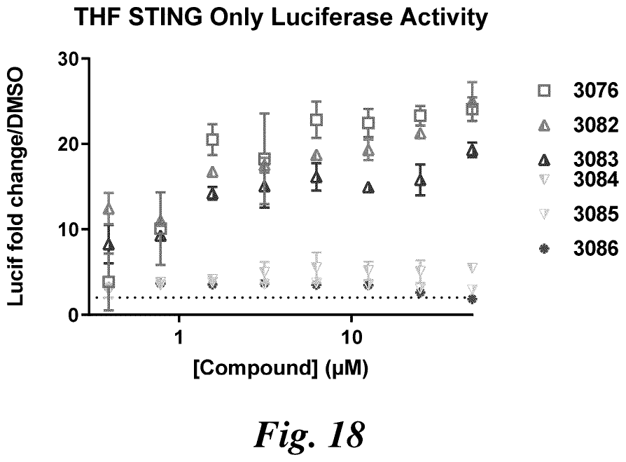

FIG. 19 shows the THF luciferase activity of compounds INI-3087, INI-3088, INI-3089, INI-3090, INI-3091, INI-3092, and INI-3069;

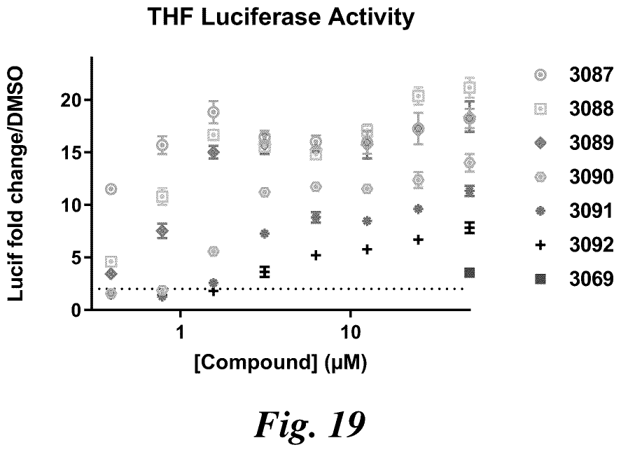

FIG. 20 shows the THF STING only luciferase activity of compounds INI-3087, INI-3088, INI-3089, INI-3090, INI-3091, and INI-3092;

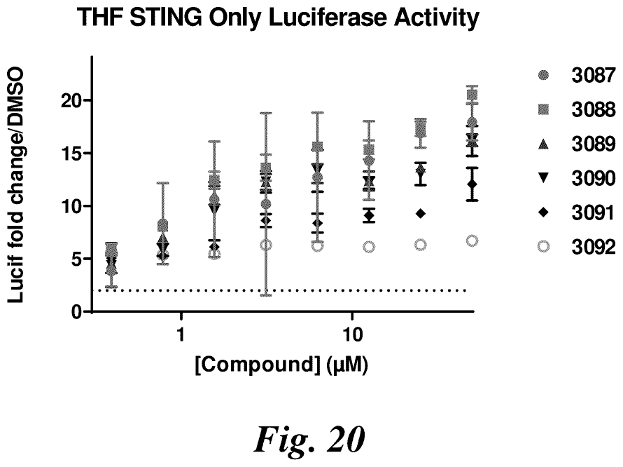

FIG. 21 shows the THF STING KO luciferase activity of compounds INI-3087, INI-3088, INI-3089, INI-3090, INI-3091, INI-3092, and IFNβ;

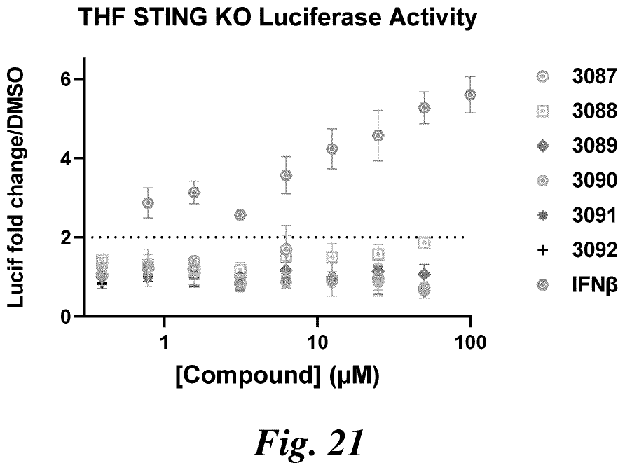

FIG. 22 shows the THF STING luciferase activity of compounds INI-3087, INI-3088, INI-3089, INI-3090, INI-3091, and INI-3092;

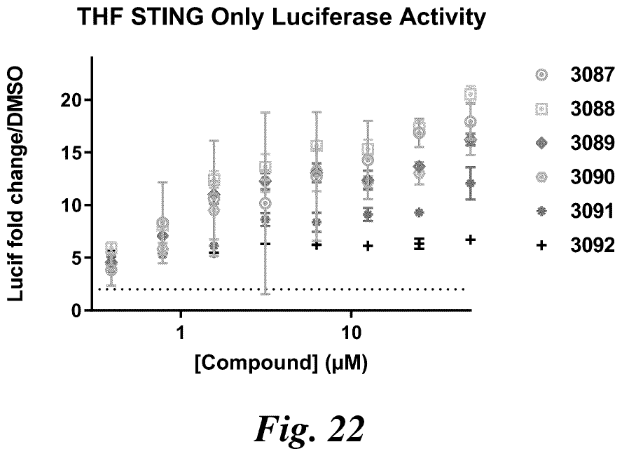

FIG. 23 shows the THF luciferase activity of compounds INI-3093, INI-3094, INI-3095, INI-3096, INI-3103, INI-3104, and INI-3069;

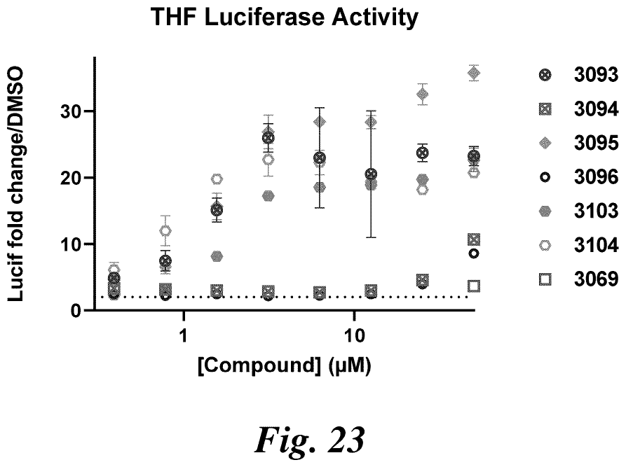

FIG. 24 shows the THF STING only luciferase activity of compounds INI-3093, INI-3094, INI-3095, INI-3096, INI-3103, and INI-3104;

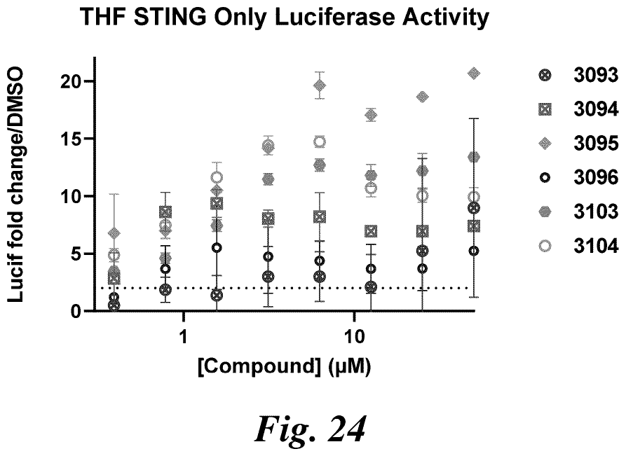

FIG. 25 shows the STING KO luciferase activity of compound INI-3093, INI-3096, INI-3103, INI-3104, and IFNβ.

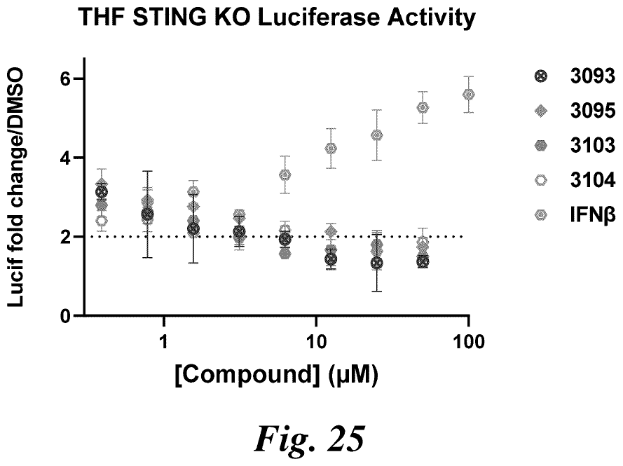

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Stimulator of Interferon Genes (Sting) Agonists

### A. Representative Compounds of Formula (I)

In some embodiments, the presently disclosed subject matter provides a compound of formula (I):

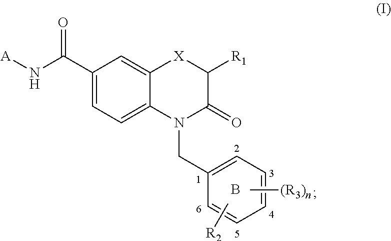

wherein:
n is 1 or 2;
X is selected from S, O, NRₓ, and CRₓ₁Rₓ₂, wherein Rₓ, Rₓ₁, and Rₓ₂ are each independently selected from H and C₁-C₄ alkyl;
A is:

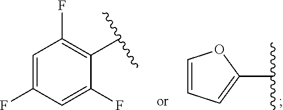 [SMILES: *c1c(F)cc(F)cc1F ; *c1ccco1]

R₁ is H or C₁-C₄ alkyl; R₂ is selected from —OR₄, —NR₅R₆, and —NO₂, wherein R₄ is H or C₁-C₂₀ alkyl and R₅ and R₆ are each independently H or C₁-C₄ alkyl; each R₃ can be the same or different and are each halogen; wherein: (i) if X is S, A is 2,4,6-trifluorophenyl, n is 2, and R₃ is F in the 2 and 6 position of ring B, then R₄ cannot be H or C₁ alkyl; or (ii) if X is S, A is 2,4,6-trifluorophenyl, n is 2, and R₃ is F in the 2 position of ring B, then R₄ cannot be C₁ alkyl; and pharmaceutically acceptable salts thereof.

Further, a structure represented generally by the formula:

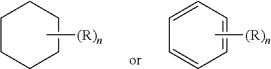

as used herein refers to a ring structure comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 2 would comprise compound groups including, but not limited to:

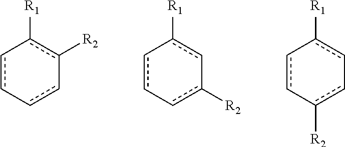

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol ( [symbol] ) denotes the point of attachment of a moiety to the remainder of the molecule.

As used herein, the term “alkyl” refers to a univalent group derived from an alkane by removal of a hydrogen atom from any carbon atom resulting in a substituent group with a formula of —CₙH₂ₙ₊₁. An alkyl group derived by removal of a hydrogen atom from a terminal carbon atom of an unbranched alkane form a subclass of normal alkyl (n-alkyl) groups having a formula —H(CH₂)ₙ. The groups RCH₂, R₂CH(R≠H), and R₃C(R≠H) are primary, secondary and tertiary alkyl groups, respectively.

The number of carbon atoms designated in the alkyl group (i.e., C₁-C₁₀ means one to ten carbons, including 1, 2, 3, 4, 5, 6, 7, 8, 9, and 10 carbons). In particular embodiments, the term “alkyl” refers to C₁-C₂₀ inclusive, including 1, 2, 3, 4, 5, 6, 7, 8, 9, 10, 11, 12, 13, 14, 15, 16, 17, 18, 19, and 20 carbons. An alkyl group can be linear (i.e., “straight-chain”) or branched. More particularly, as used herein the term “C₁-C₄ alkyl” refers to an alkyl group having 1, 2, 3, or 4 carbon atoms. Representative C₁-C₄ alkyl groups include methyl, ethyl, n-propyl, isopropyl, n-butyl, isobutyl, sec-butyl, tert-butyl. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁-8 alkyl), e.g., 1, 2, 3, 4, 5, 6, 7, or 8 carbon atoms.

In some embodiments, the alkyl group can be substituted. As used herein, the term “substituted alkyl” includes alkyl groups, as defined herein, in which one or more atoms of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, cyano, and mercapto.

The terms “halo,” “halide,” or “halogen” as used herein refer to fluoro, chloro, bromo, and iodo groups. In some embodiments, the halogen is F. In some embodiments, the halogen is Cl.

In particular embodiments, the compound of formula (I) is a compound of formula (I′):

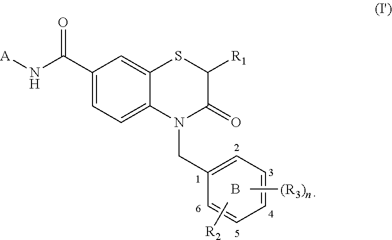

In certain embodiments, the compound of formula (I′) is a compound of formula (Ia):

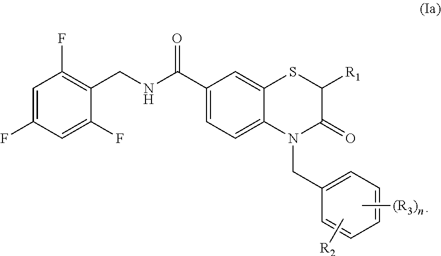

In certain embodiments, the compound of formula (Ia) is a compound of formula (Ia-i):

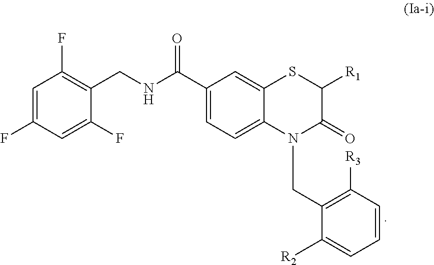

In particular embodiments of a compound of formula (Ia-i). R₂ is —OR₄. In particular embodiments. R is H. In such embodiments, the compound of formula (Ia-i) is selected from:

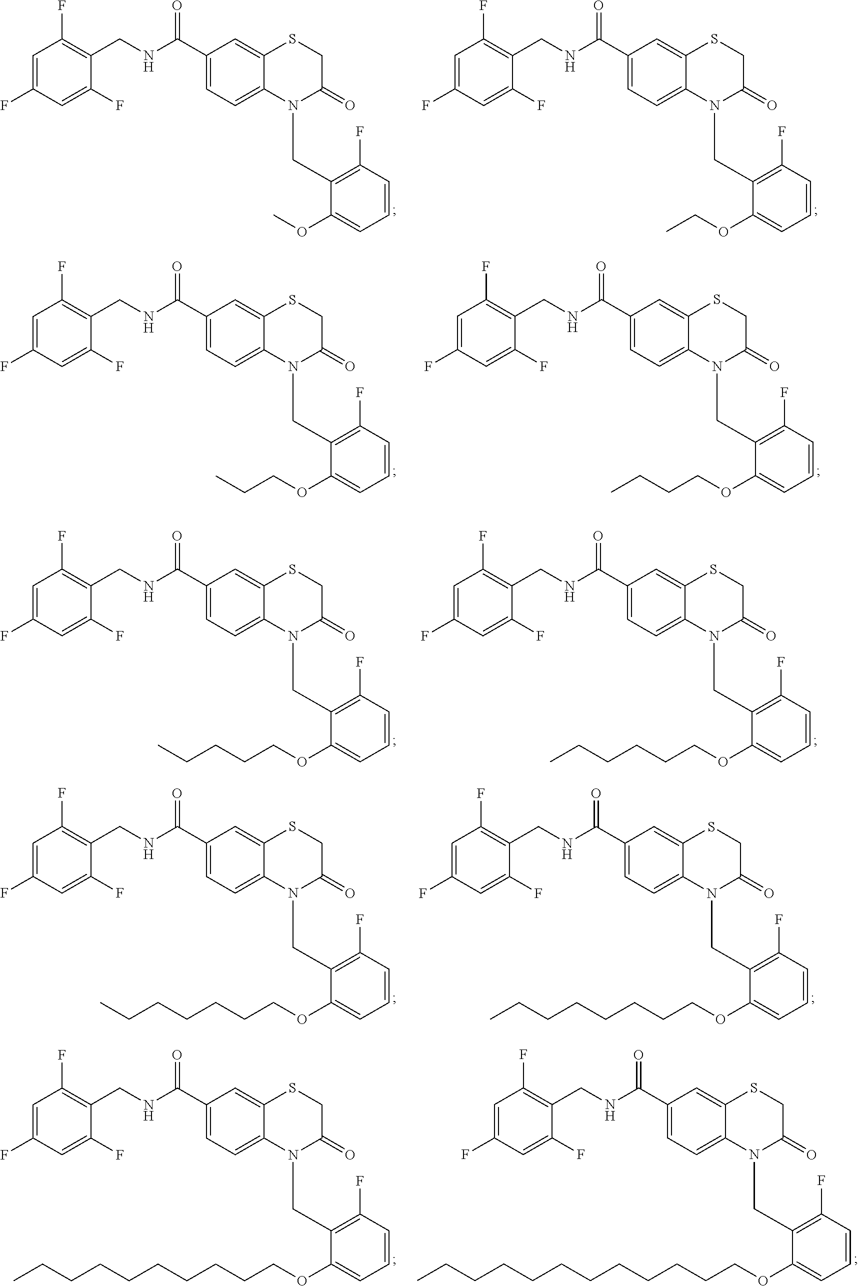

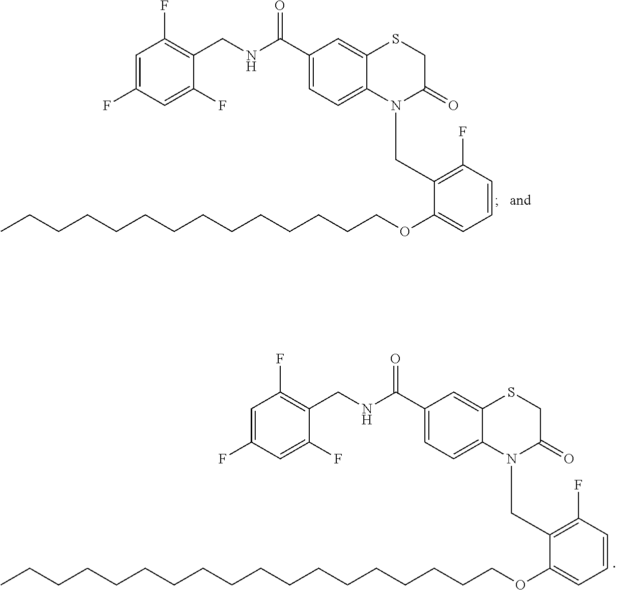 [SMILES: CCCCCCCCCCCCCCOc1cccc(F)c1CN1C(=O)CSc2cc(C(=O)NCc3c(F)cc(F)cc3F)ccc21 ; CCCCCCCCCCCCCCCCCCOc1cccc(F)c1CN1C(=O)CSc2cc(C(=O)NCc3c(F)cc(F)cc3F)ccc21]

In particular embodiments of the compound of formula (Ia-i). R₁ is C₁-C₄ alkyl, in such embodiments, the compound of formula (Ia-i) is selected from:

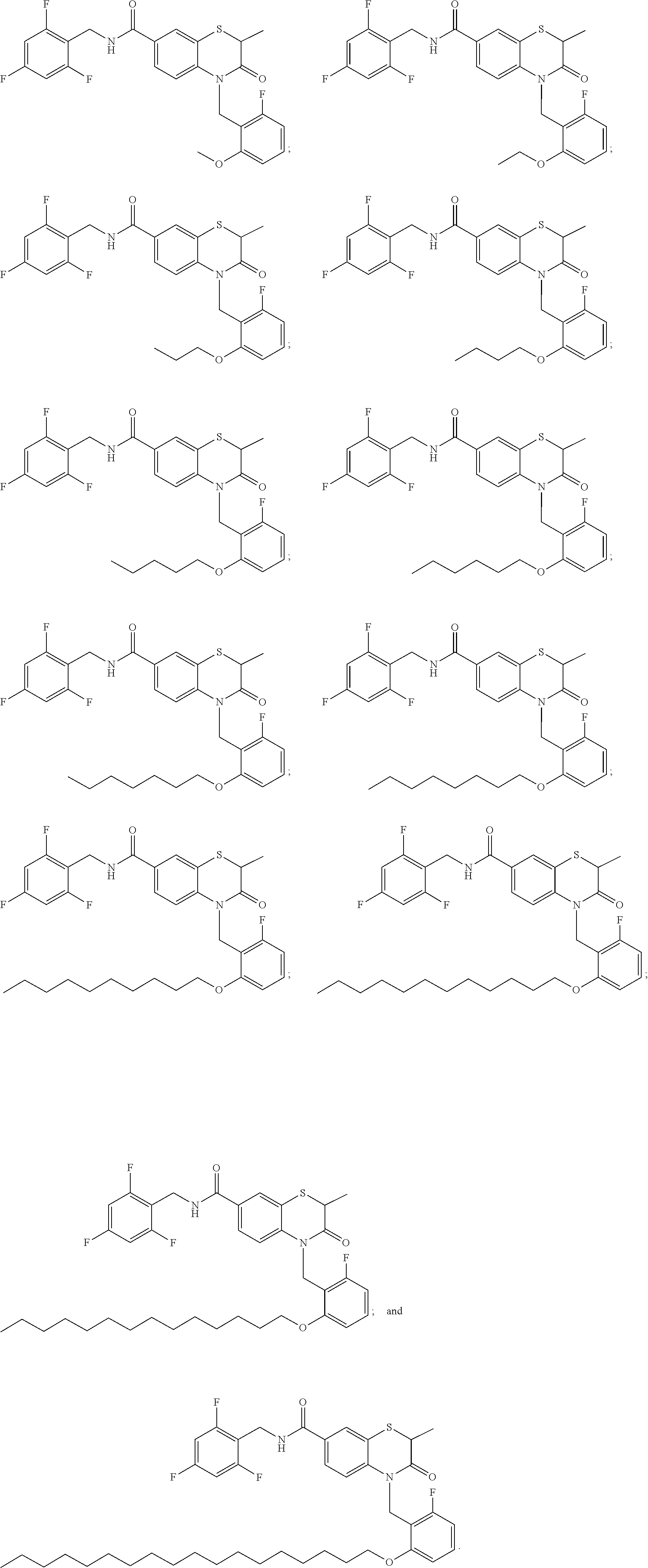

In certain embodiments, the compound of formula (Ia) is a compound of formula (Ia-ii):

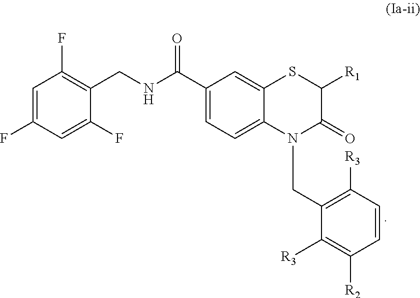

In particular embodiments of the compound of formula (Ia-ii). R₂ is —OR₄. In particular embodiments. R₁ is H. In such embodiments, the compound of formula (Ia-ii) is selected from:

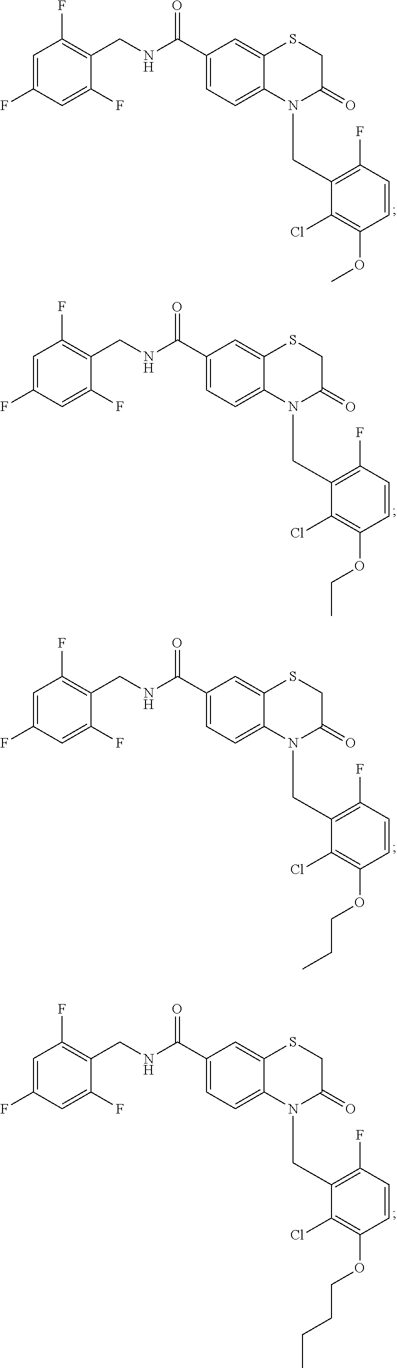 [SMILES: COc1ccc(F)c(CN2C(=O)CSc3cc(C(=O)NCc4c(F)cc(F)cc4F)ccc32)c1Cl ; CCOc1ccc(F)c(CN2C(=O)CSc3cc(C(=O)NCc4c(F)cc(F)cc4F)ccc32)c1Cl ; CCCOc1ccc(F)c(CN2C(=O)CSc3cc(C(=O)NCc4c(F)cc(F)cc4F)ccc32)c1Cl ; CCCCOc1ccc(F)c(CN2C(=O)CSc3cc(C(=O)NCc4c(F)cc(F)cc4F)ccc32)c1Cl]

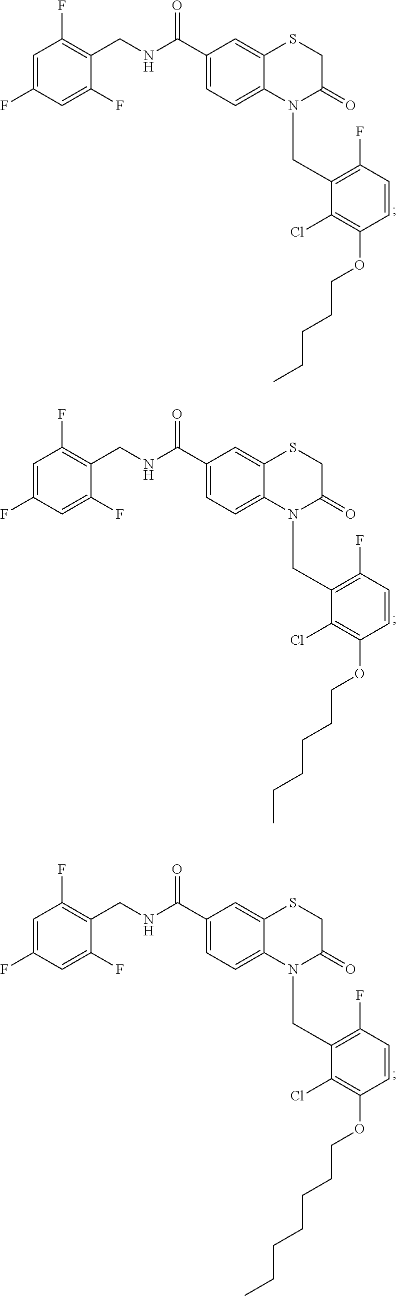 [SMILES: CCCCCOc1ccc(F)c(CN2C(=O)CSc3cc(C(=O)NCc4c(F)cc(F)cc4F)ccc32)c1Cl ; CCCCCCOc1ccc(F)c(CN2C(=O)CSc3cc(C(=O)NCc4c(F)cc(F)cc4F)ccc32)c1Cl ; CCCCCCCOc1ccc(F)c(CN2C(=O)CSc3cc(C(=O)NCc4c(F)cc(F)cc4F)ccc32)c1Cl]

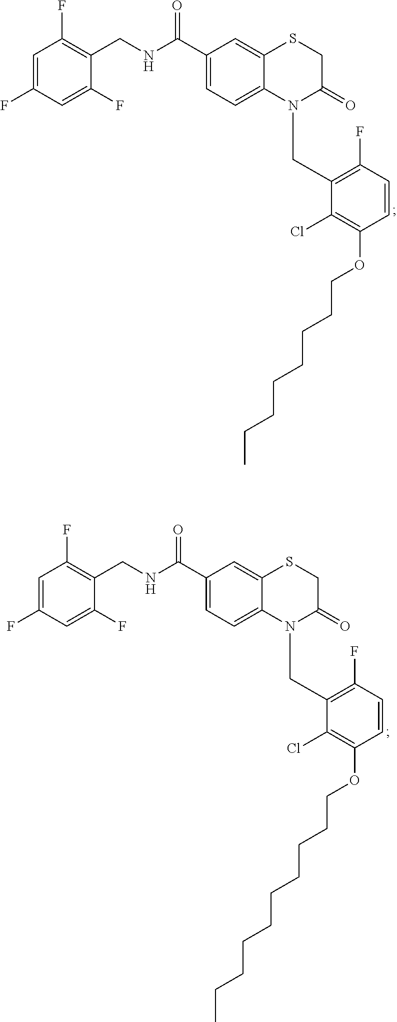 [SMILES: CCCCCCCCOc1ccc(F)c(CN2C(=O)CSc3cc(C(=O)NCc4c(F)cc(F)cc4F)ccc32)c1Cl ; CCCCCCCCCCOc1ccc(F)c(CN2C(=O)CSc3cc(C(=O)NCc4c(F)cc(F)cc4F)ccc32)c1Cl]

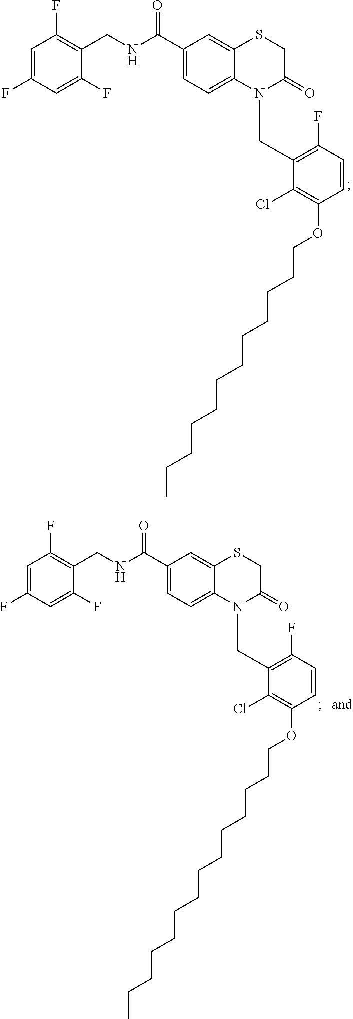

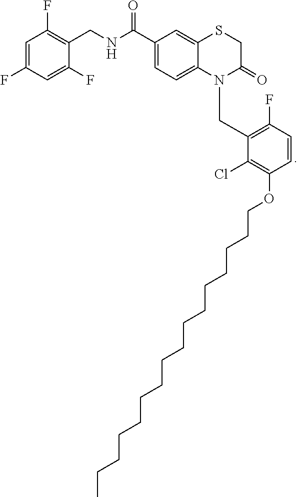 [SMILES: CCCCCCCCCCCCCCCCOc1ccc(F)c(CN2C(=O)CSc3cc(C(=O)NCc4c(F)cc(F)cc4F)ccc32)c1Cl]

In certain embodiments of the compound of formula (Ia-ii). R₁ is C₁-C₄ alkyl. In such embodiments, the compound of formula (Ia-ii) is selected from:

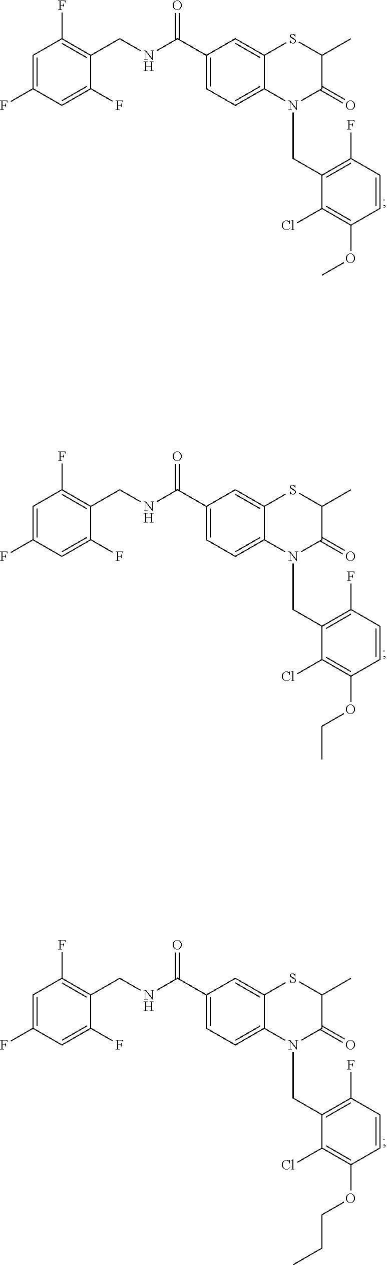 [SMILES: COc1ccc(F)c(CN2C(=O)C(C)Sc3cc(C(=O)NCc4c(F)cc(F)cc4F)ccc32)c1Cl ; CCOc1ccc(F)c(CN2C(=O)C(C)Sc3cc(C(=O)NCc4c(F)cc(F)cc4F)ccc32)c1Cl ; CCCOc1ccc(F)c(CN2C(=O)C(C)Sc3cc(C(=O)NCc4c(F)cc(F)cc4F)ccc32)c1Cl]

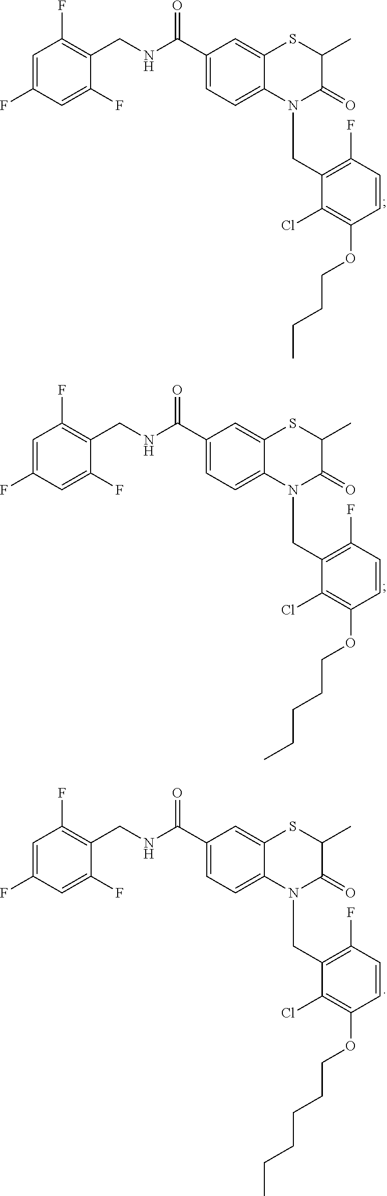 [SMILES: CCCCOc1ccc(F)c(CN2C(=O)C(C)Sc3cc(C(=O)NCc4c(F)cc(F)cc4F)ccc32)c1Cl ; CCCCCOc1ccc(F)c(CN2C(=O)C(C)Sc3cc(C(=O)NCc4c(F)cc(F)cc4F)ccc32)c1Cl ; CCCCCCOc1ccc(F)c(CN2C(=O)C(C)Sc3cc(C(=O)NCc4c(F)cc(F)cc4F)ccc32)c1Cl]

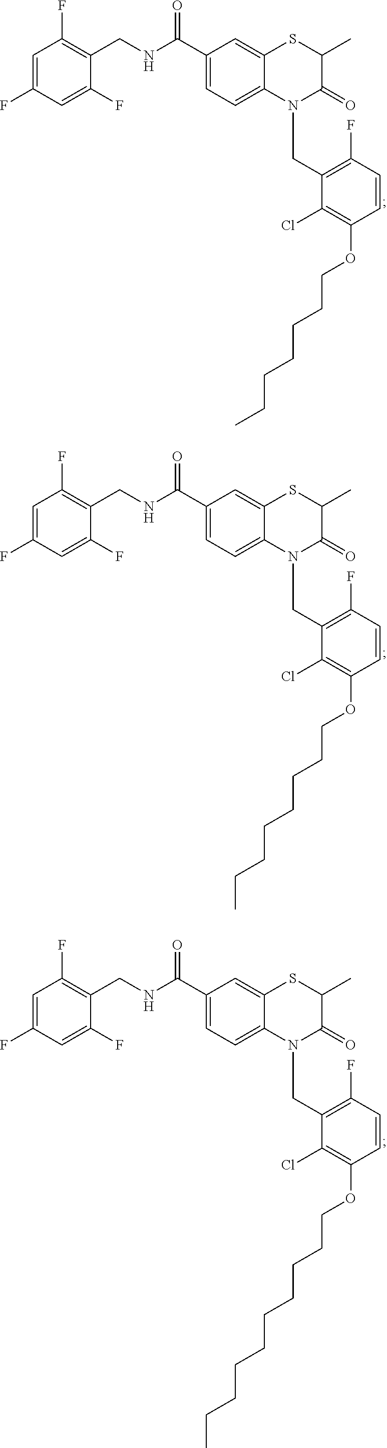 [SMILES: CCCCCCCOc1ccc(F)c(CN2C(=O)C(C)Sc3cc(C(=O)NCc4c(F)cc(F)cc4F)ccc32)c1Cl ; CCCCCCCCOc1ccc(F)c(CN2C(=O)C(C)Sc3cc(C(=O)NCc4c(F)cc(F)cc4F)ccc32)c1Cl ; CCCCCCCCCCOc1ccc(F)c(CN2C(=O)C(C)Sc3cc(C(=O)NCc4c(F)cc(F)cc4F)ccc32)c1Cl]

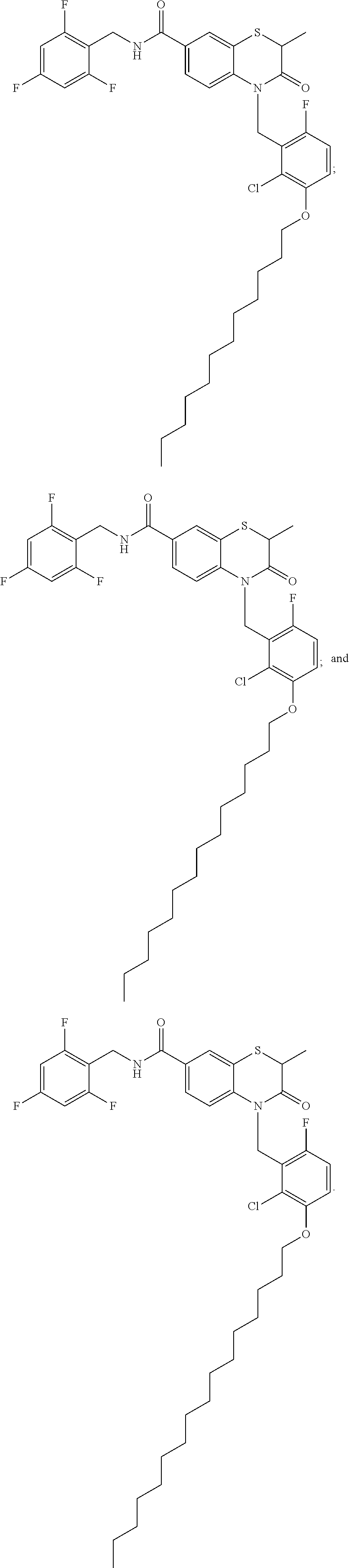

In certain embodiments, the compound of formula (Ia) is a compound of formula (Ia-iii):

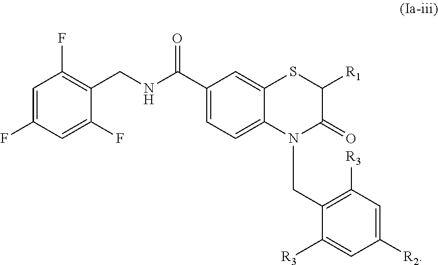

In particular embodiments of a compound of formula (Ia-iii), R₂ is —OR₄. In certain embodiments. R₁ is H. In such embodiments, the compound of formula (Ia-iii) is selected from:

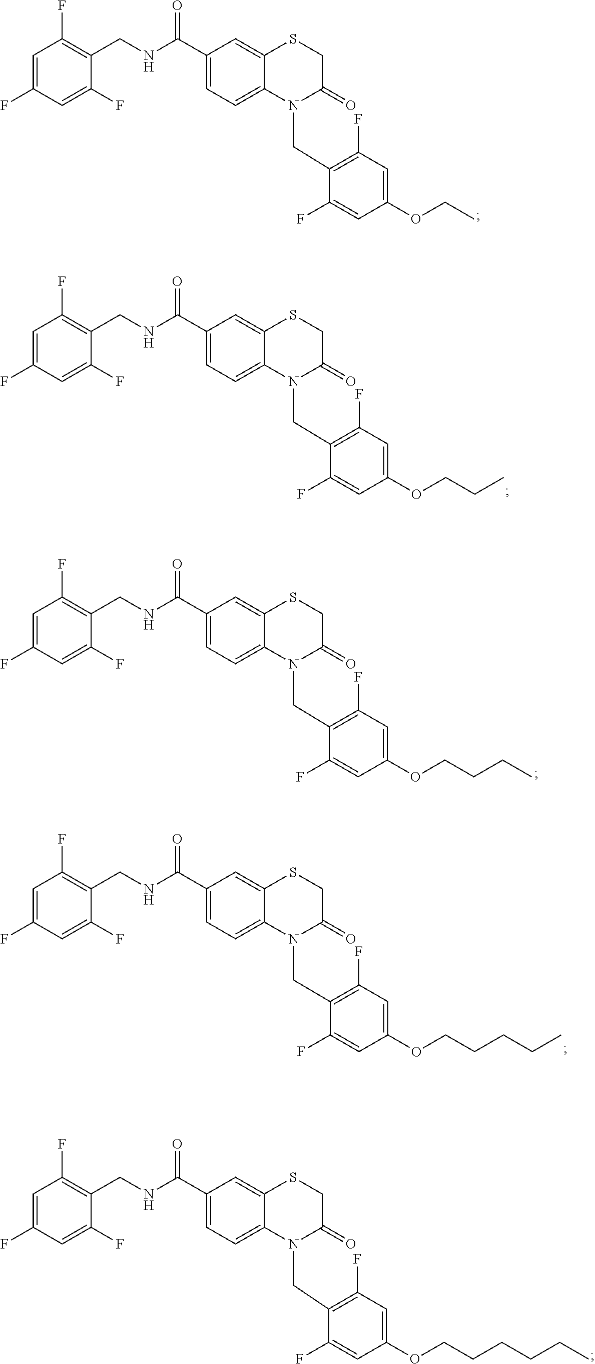 [SMILES: CCOc1cc(F)c(CN2C(=O)CSc3cc(C(=O)NCc4c(F)cc(F)cc4F)ccc32)c(F)c1 ; CCCOc1cc(F)c(CN2C(=O)CSc3cc(C(=O)NCc4c(F)cc(F)cc4F)ccc32)c(F)c1 ; CCCCOc1cc(F)c(CN2C(=O)CSc3cc(C(=O)NCc4c(F)cc(F)cc4F)ccc32)c(F)c1 ; CCCCCOc1cc(F)c(CN2C(=O)CSc3cc(C(=O)NCc4c(F)cc(F)cc4F)ccc32)c(F)c1 ; CCCCCCOc1cc(F)c(CN2C(=O)CSc3cc(C(=O)NCc4c(F)cc(F)cc4F)ccc32)c(F)c1]

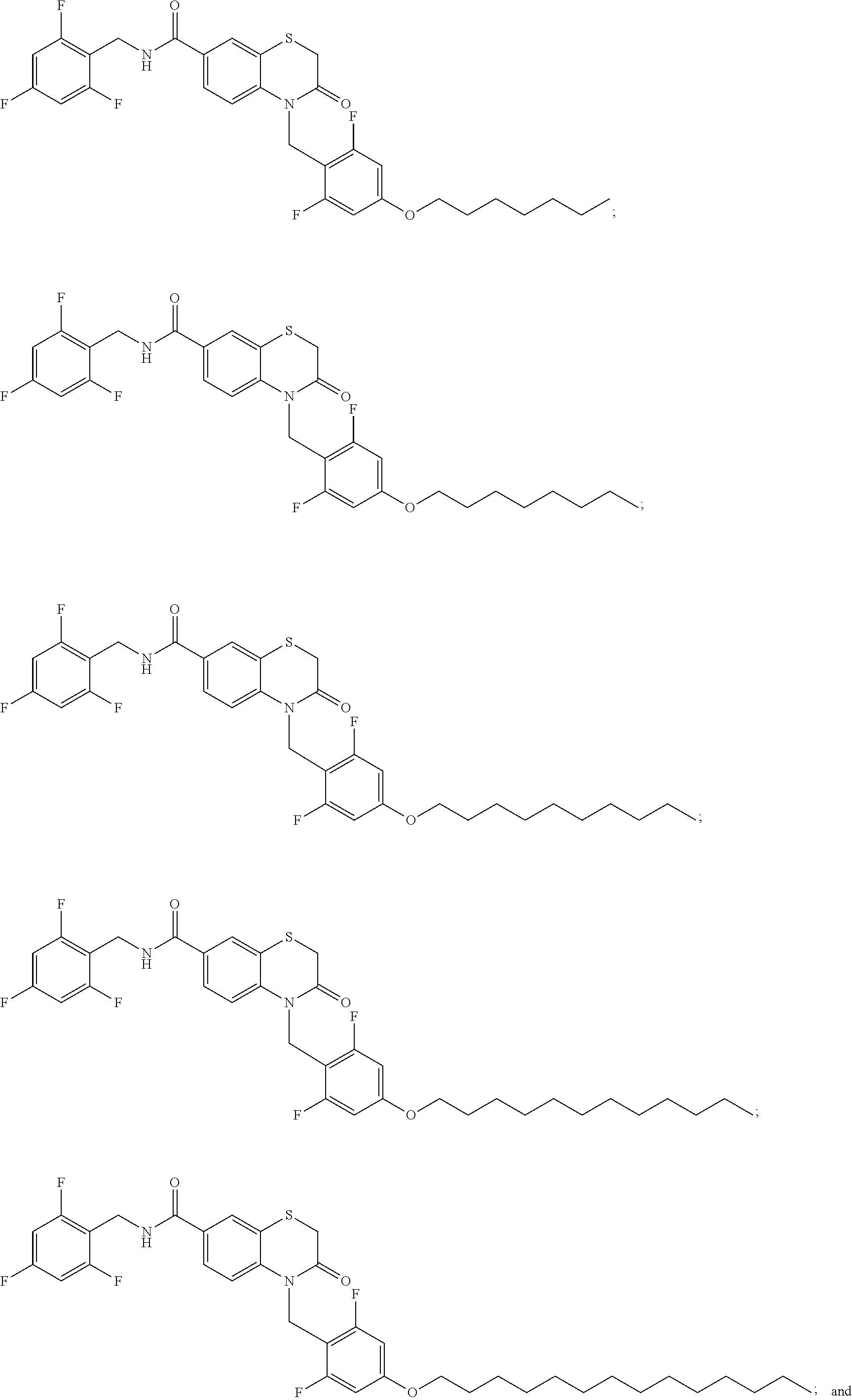 [SMILES: CCCCCCCOc1cc(F)c(CN2C(=O)CSc3cc(C(=O)NCc4c(F)cc(F)cc4F)ccc32)c(F)c1 ; CCCCCCCCOc1cc(F)c(CN2C(=O)CSc3cc(C(=O)NCc4c(F)cc(F)cc4F)ccc32)c(F)c1 ; CCCCCCCCCCOc1cc(F)c(CN2C(=O)CSc3cc(C(=O)NCc4c(F)cc(F)cc4F)ccc32)c(F)c1 ; CCCCCCCCCCCCOc1cc(F)c(CN2C(=O)CSc3cc(C(=O)NCc4c(F)cc(F)cc4F)ccc32)c(F)c1 ; CCCCCCCCCCCCCCOc1cc(F)c(CN2C(=O)CSc3cc(C(=O)NCc4c(F)cc(F)cc4F)ccc32)c(F)c1]

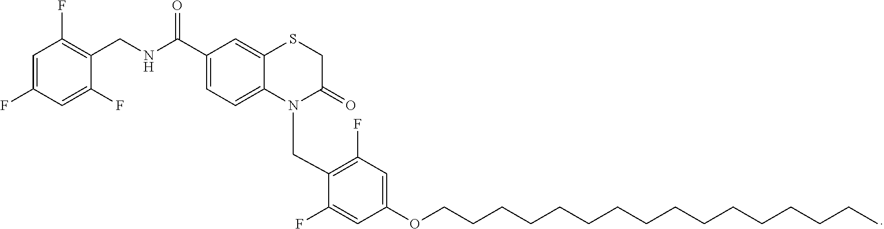 [SMILES: CCCCCCCCCCCCCCCCOc1cc(F)c(CN2C(=O)CSc3cc(C(=O)NCc4c(F)cc(F)cc4F)ccc32)c(F)c1]

In particular embodiments of the compound of formula (Ia-iii), R₁ is C₁-C₄ alkyl. In such embodiments, the compound of formula (Ia-iii) is selected from:

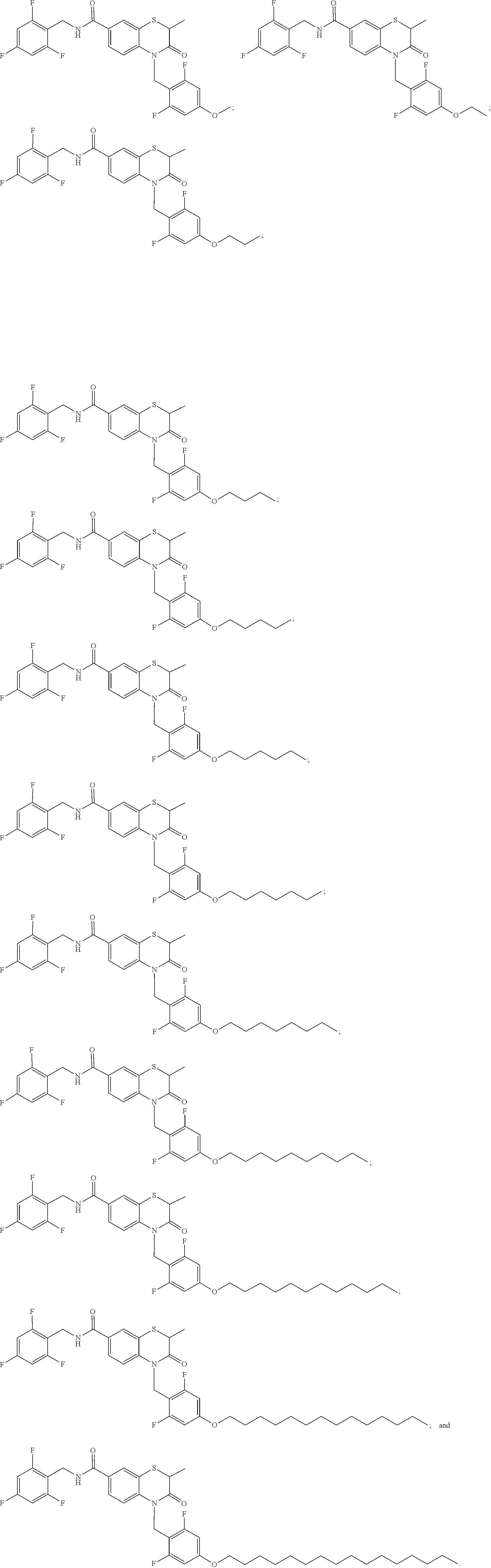

In certain embodiments of the compound of formula (I), R₂ is —NR₅R₆ or —NO₂. In certain embodiments of the compound of formula (I-a), R₂ is —NR₅R₆ or —NO₂. In such embodiments, the compound of formula (I-a) is selected from:

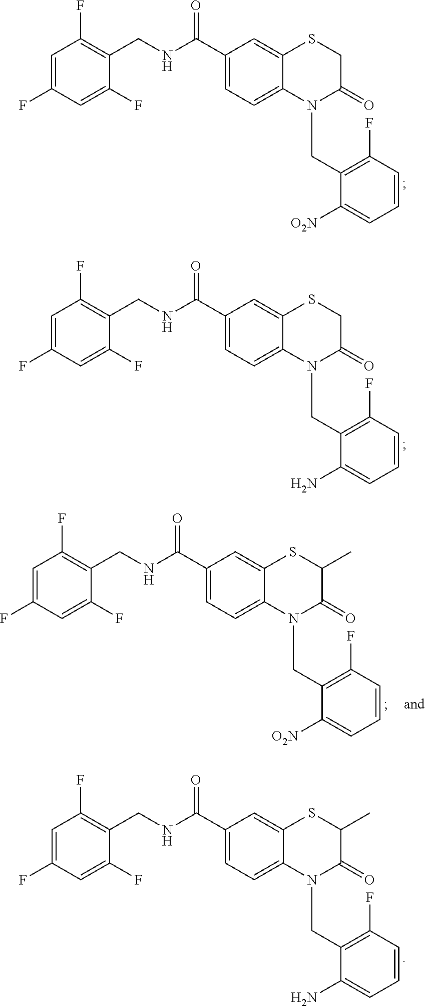 [SMILES: O=C(NCc1c(F)cc(F)cc1F)c1ccc2c(c1)SCC(=O)N2Cc1c(F)cccc1[N+](=O)[O-] ; Nc1cccc(F)c1CN1C(=O)CSc2cc(C(=O)NCc3c(F)cc(F)cc3F)ccc21 ; CC1Sc2cc(C(=O)NCc3c(F)cc(F)cc3F)ccc2N(Cc2c(F)cccc2[N+](=O)[O-])C1=O ; CC1Sc2cc(C(=O)NCc3c(F)cc(F)cc3F)ccc2N(Cc2c(N)cccc2F)C1=O]

In certain embodiments, the compound of formula (I) is a compound of formula (Ib):

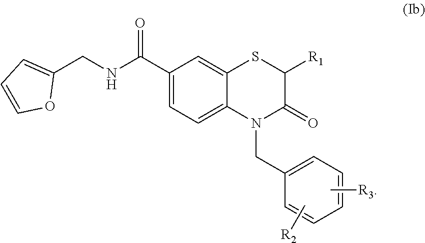

In particular embodiments, the compound of formula (Ib) is a compound of formula (Ib-i):

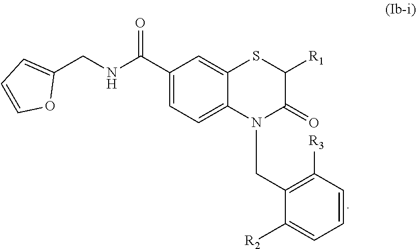

In certain embodiments of the compound of formula (Ib-i), R₂ is —OR₄. In certain embodiments of the compound of formula (Ib-i), R₁ is H. In such embodiments, the compound of formula (Ib-i) is selected from:

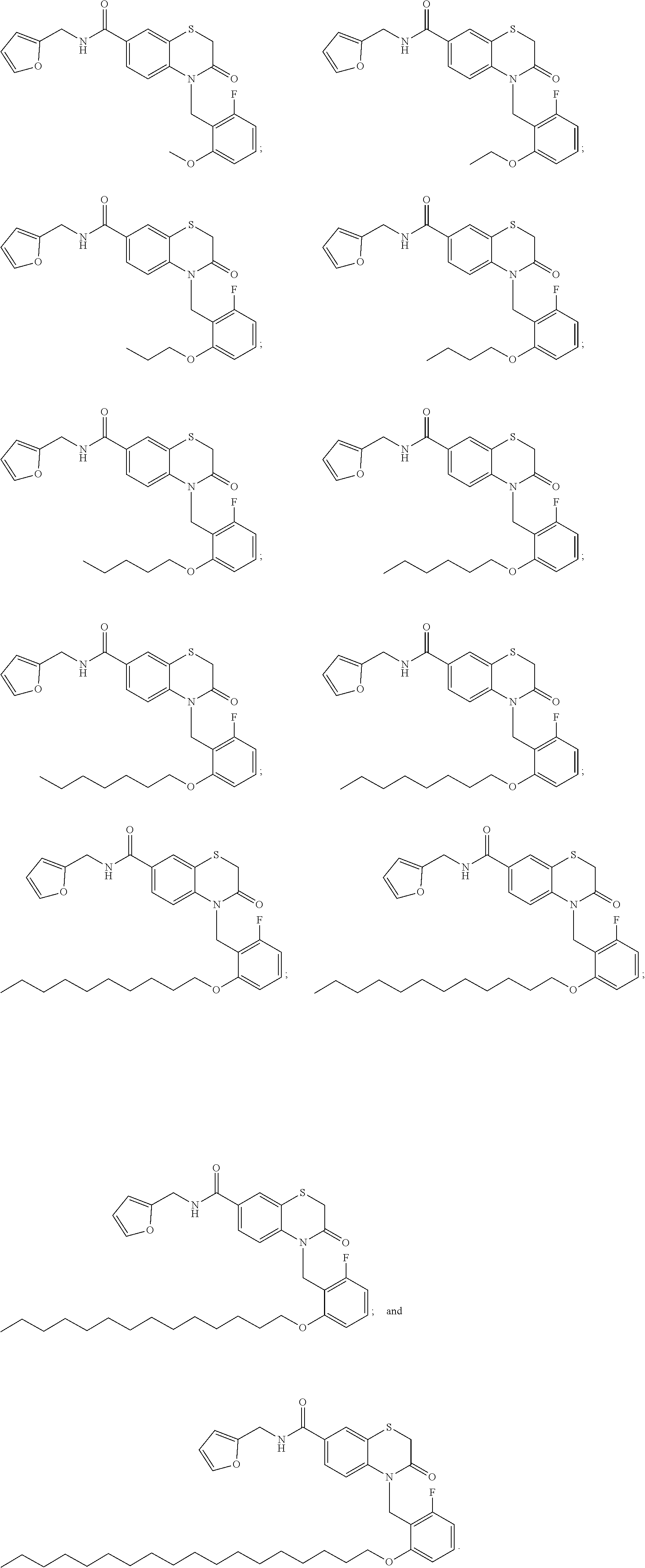

In certain embodiments of the compound of formula (Ib-i). R₁ is C₁-C₄ alkyl. In such embodiments, the compound of formula (Ib-i) is selected from:

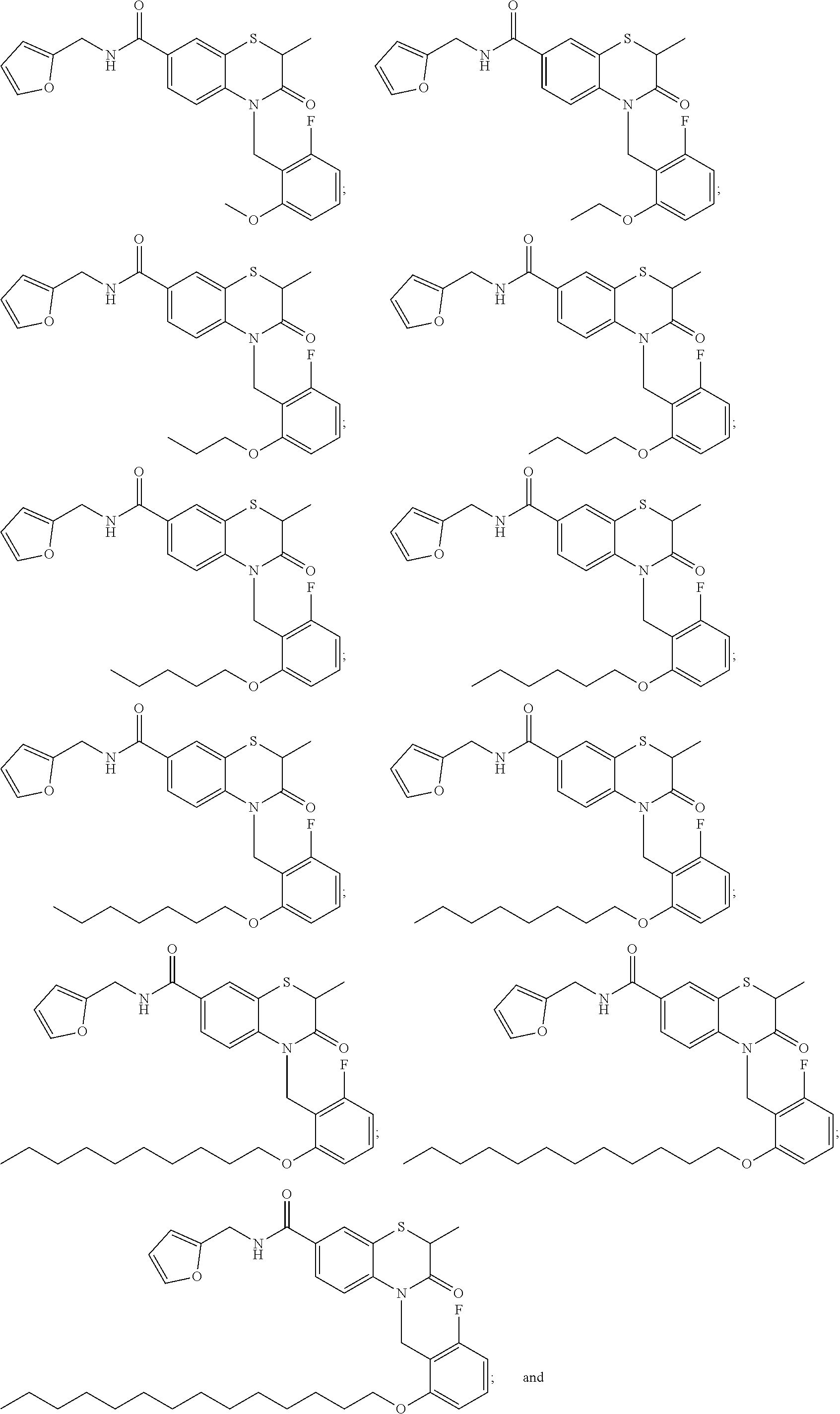

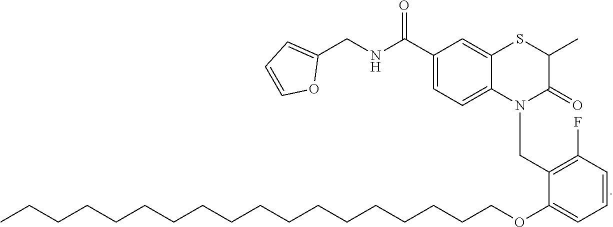 [SMILES: CCCCCCCCCCCCCCCCCCOc1cccc(F)c1CN1C(=O)C(C)Sc2cc(C(=O)NCc3ccco3)ccc21]

In certain embodiments, the compound of formula (Ib) is a compound of formula (Ib-ii):

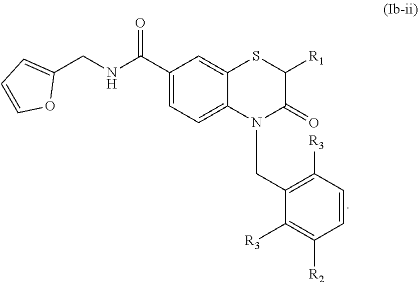

In certain embodiments of the compound of formula (Ib-ii). R₂ is —OR₄. In certain embodiments of the compound of formula (Ib-ii). R₁ is H. In such embodiments, the compound of formula (Ib-ii) is selected from:

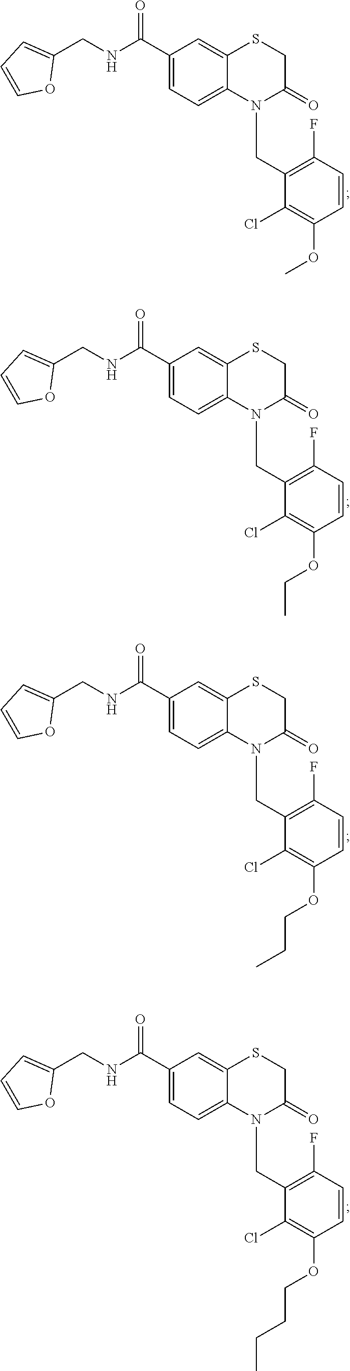 [SMILES: COc1ccc(F)c(CN2C(=O)CSc3cc(C(=O)NCc4ccco4)ccc32)c1Cl ; CCOc1ccc(F)c(CN2C(=O)CSc3cc(C(=O)NCc4ccco4)ccc32)c1Cl ; CCCOc1ccc(F)c(CN2C(=O)CSc3cc(C(=O)NCc4ccco4)ccc32)c1Cl ; CCCCOc1ccc(F)c(CN2C(=O)CSc3cc(C(=O)NCc4ccco4)ccc32)c1Cl]

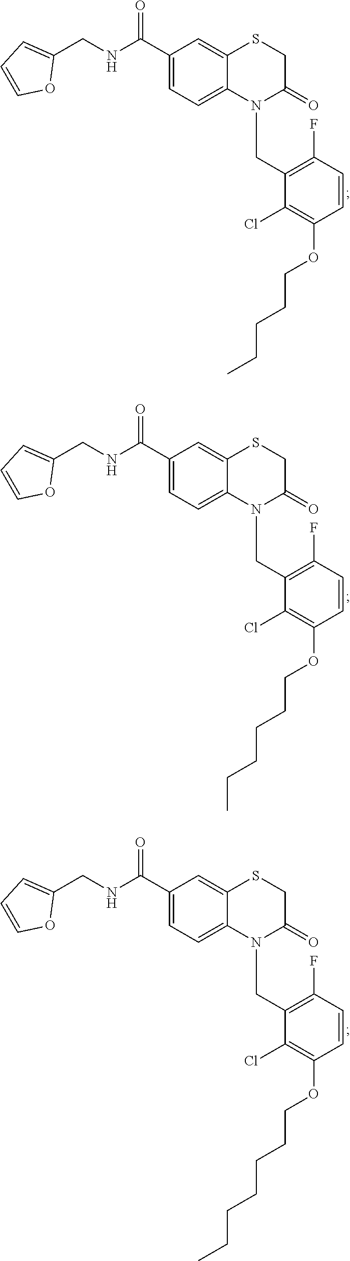 [SMILES: CCCCCOc1ccc(F)c(CN2C(=O)CSc3cc(C(=O)NCc4ccco4)ccc32)c1Cl ; CCCCCCOc1ccc(F)c(CN2C(=O)CSc3cc(C(=O)NCc4ccco4)ccc32)c1Cl ; CCCCCCCOc1ccc(F)c(CN2C(=O)CSc3cc(C(=O)NCc4ccco4)ccc32)c1Cl]

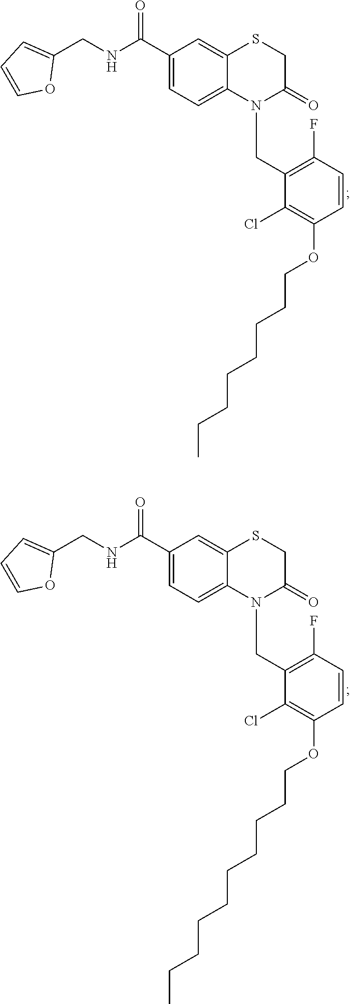 [SMILES: CCCCCCCCOc1ccc(F)c(CN2C(=O)CSc3cc(C(=O)NCc4ccco4)ccc32)c1Cl ; CCCCCCCCCCOc1ccc(F)c(CN2C(=O)CSc3cc(C(=O)NCc4ccco4)ccc32)c1Cl]

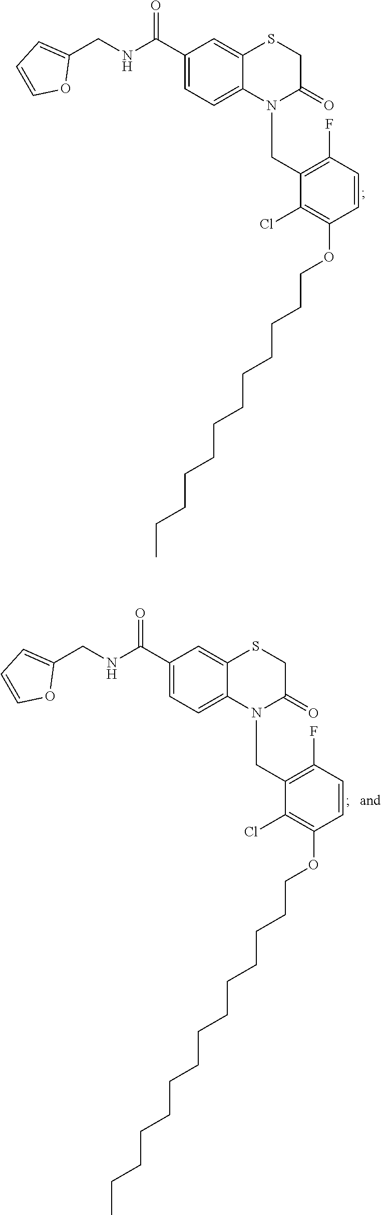 [SMILES: CCCCCCCCCCCCOc1ccc(F)c(CN2C(=O)CSc3cc(C(=O)NCc4ccco4)ccc32)c1Cl ; CCCCCCCCCCCCCCOc1ccc(F)c(CN2C(=O)CSc3cc(C(=O)NCc4ccco4)ccc32)c1Cl]

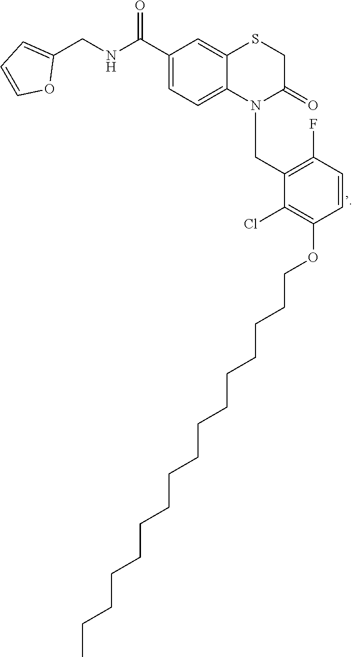 [SMILES: CCCCCCCCCCCCCCCCOc1ccc(F)c(CN2C(=O)CSc3cc(C(=O)NCc4ccco4)ccc32)c1Cl]

In certain embodiments of the compound of formula (Ib-ii). R₁ is C₁-C₄ alkyl. In such embodiments, the compound of formula (Ib-ii) is selected from:

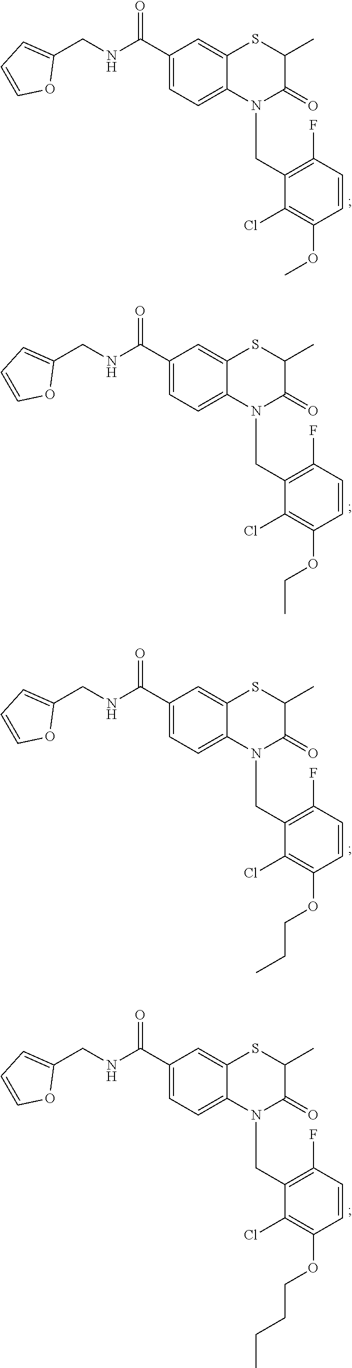 [SMILES: COc1ccc(F)c(CN2C(=O)C(C)Sc3cc(C(=O)NCc4ccco4)ccc32)c1Cl ; CCOc1ccc(F)c(CN2C(=O)C(C)Sc3cc(C(=O)NCc4ccco4)ccc32)c1Cl ; CCCOc1ccc(F)c(CN2C(=O)C(C)Sc3cc(C(=O)NCc4ccco4)ccc32)c1Cl ; CCCCOc1ccc(F)c(CN2C(=O)C(C)Sc3cc(C(=O)NCc4ccco4)ccc32)c1Cl]

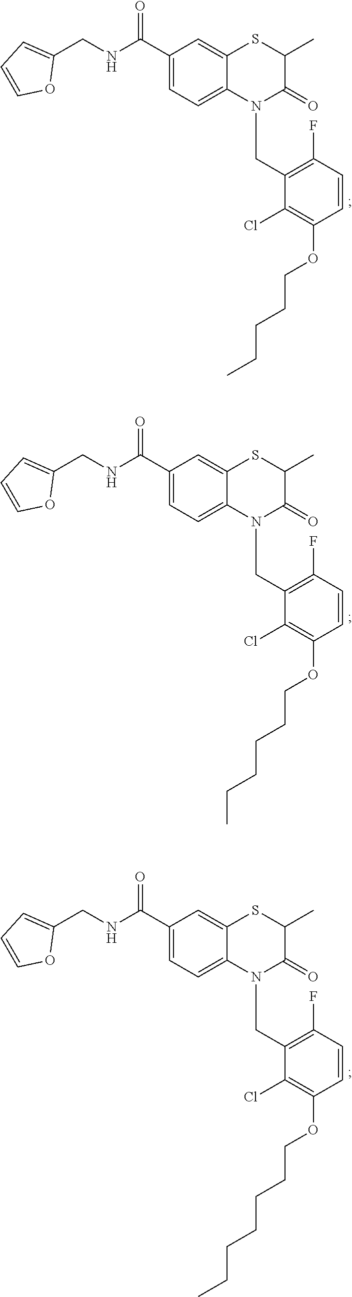 [SMILES: CCCCCOc1ccc(F)c(CN2C(=O)C(C)Sc3cc(C(=O)NCc4ccco4)ccc32)c1Cl ; CCCCCCOc1ccc(F)c(CN2C(=O)C(C)Sc3cc(C(=O)NCc4ccco4)ccc32)c1Cl ; CCCCCCCOc1ccc(F)c(CN2C(=O)C(C)Sc3cc(C(=O)NCc4ccco4)ccc32)c1Cl]

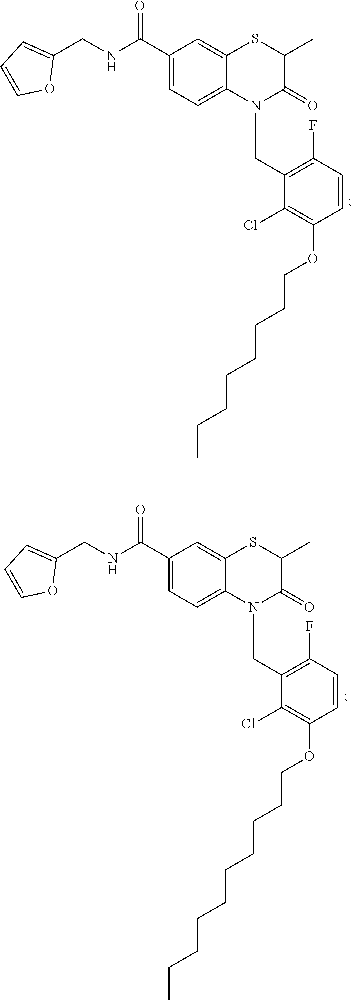 [SMILES: CCCCCCCCOc1ccc(F)c(CN2C(=O)C(C)Sc3cc(C(=O)NCc4ccco4)ccc32)c1Cl ; CCCCCCCCCCOc1ccc(F)c(CN2C(=O)C(C)Sc3cc(C(=O)NCc4ccco4)ccc32)c1Cl]

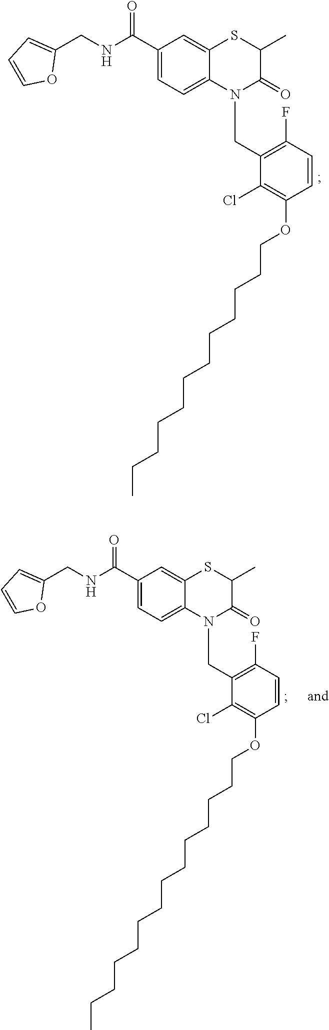 [SMILES: CCCCCCCCCCCCOc1ccc(F)c(CN2C(=O)C(C)Sc3cc(C(=O)NCc4ccco4)ccc32)c1Cl ; CCCCCCCCCCCCCCOc1ccc(F)c(CN2C(=O)C(C)Sc3cc(C(=O)NCc4ccco4)ccc32)c1Cl]

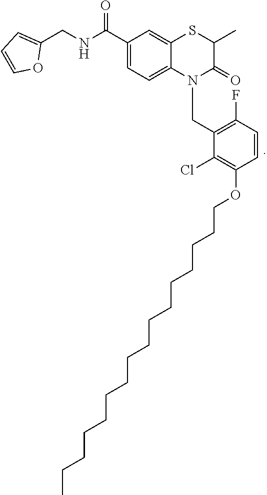 [SMILES: CCCCCCCCCCCCCCCCOc1ccc(F)c(CN2C(=O)C(C)Sc3cc(C(=O)NCc4ccco4)ccc32)c1Cl]

In certain embodiments, the compound of formula (Ib) is a compound of formula (Ia-iii):

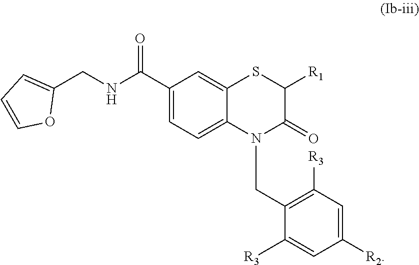

In certain embodiments of the compound of formula (Ib-iii), R₂ is —OR₄. In certain embodiments of the compound of formula (Ib-iii), R₁ is H. In such embodiments, the compound of formula (Ib-iii) is selected from:

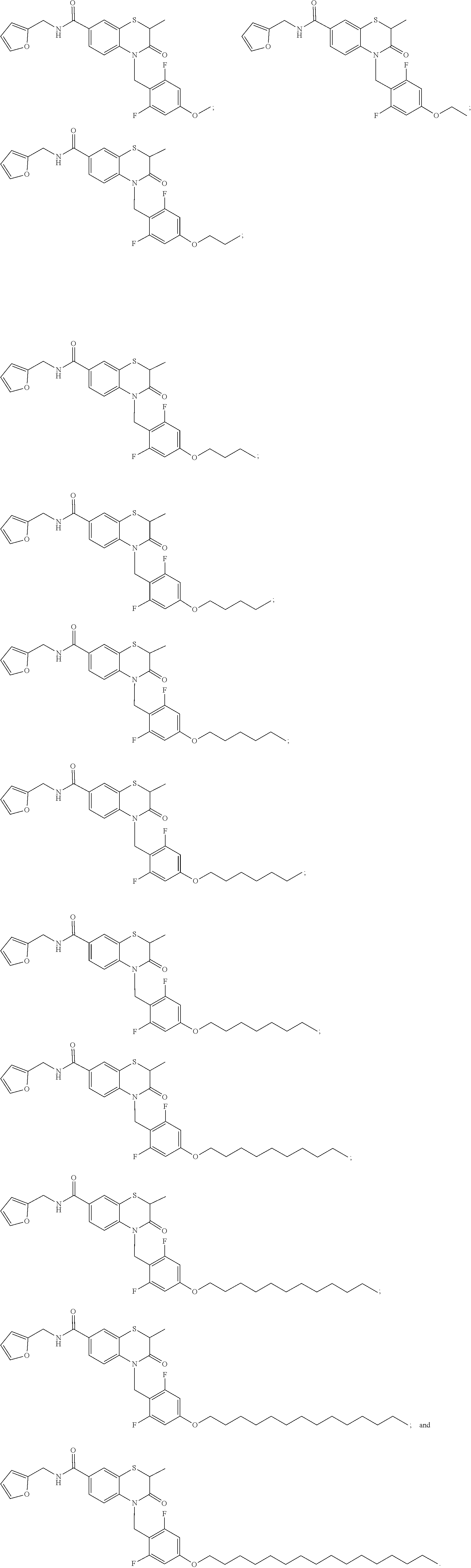

In certain embodiments of the compound of formula (Ib-iii), R₁ is C₁-C₄ alkyl. In such embodiments, the compound of formula (Ib-iii) is selected from:

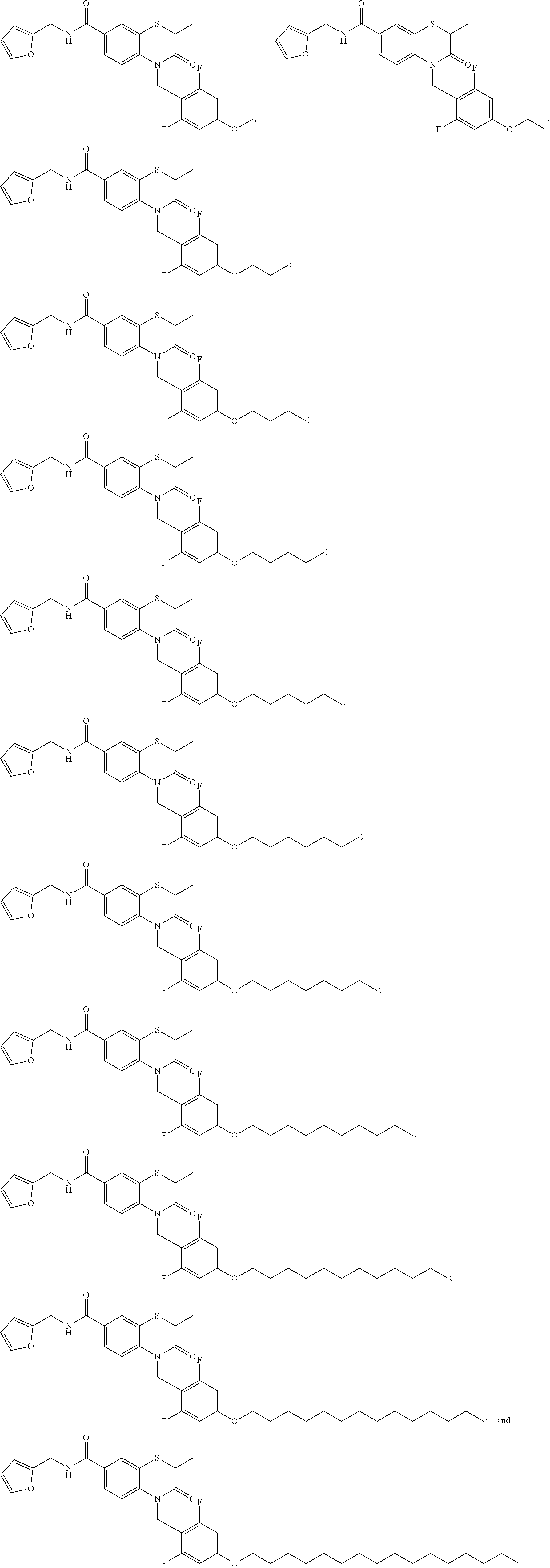

In certain embodiments of the compound of formula (I-b). R₂ is —NR₅R₆ or —NO₂. In certain embodiments of the compound of formula (I-b). R₂ is —NR₅R₆ or —NO₂. In such embodiments, the compound of formula (I-b) is selected from:

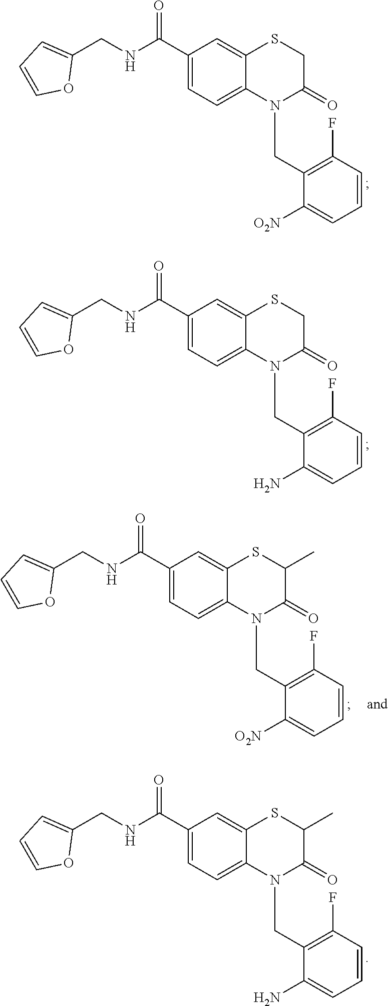 [SMILES: O=C(NCc1ccco1)c1ccc2c(c1)SCC(=O)N2Cc1c(F)cccc1[N+](=O)[O-] ; Nc1cccc(F)c1CN1C(=O)CSc2cc(C(=O)NCc3ccco3)ccc21 ; CC1Sc2cc(C(=O)NCc3ccco3)ccc2N(Cc2c(F)cccc2[N+](=O)[O-])C1=O ; CC1Sc2cc(C(=O)NCc3ccco3)ccc2N(Cc2c(N)cccc2F)C1=O]

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (1251) or carbon-14 (14° C.). All isotopic variations of the compounds of the present disclosure, whether radioactive or not, are encompassed within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

The term “protecting group” refers to chemical moieties that block some or all reactive moieties of a compound and prevent such moieties from participating in chemical reactions until the protective group is removed, for example, those moieties listed and described in T. W. Greene, P. G. M. Wuts, Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and tert-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as tert-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium (O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

Typical blocking/protecting groups include, but are not limited to the following moieties:

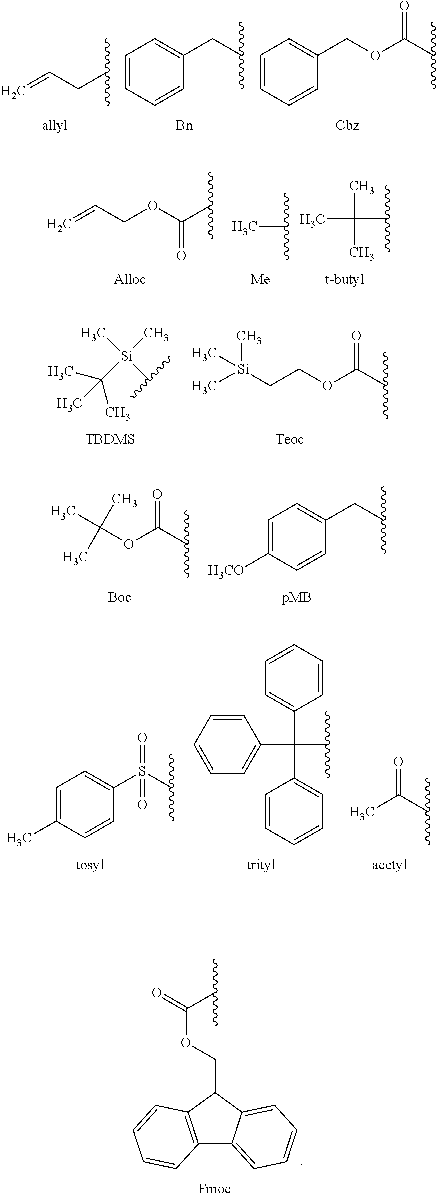

B. Methods for Treating a Disease, Disorder, or Condition Associated with STING

In some embodiments, the presently disclosed subject matter provides a method for modulating Stimulator of Interferon Genes (STING), the method comprising contacting a cell with a compound of formula (I), formula (I-a), formula (I-ai-iii), formula (I-b) and formula (I-bi-iii).

As used herein, the term “contacting” refers to placement under conditions in which direct physical association occurs, including contacting of a solid with a solid, a liquid with a liquid, a liquid with a solid, or either a liquid or a solid with a cell or tissue, whether in vitro or in vivo. Contacting can occur in vitro with isolated cells or tissue or in vivo by administering to a subject.

In some embodiments, the presently disclosed subject matter provides a method for activating or agonizing STING, the method comprising contacting a cell with a compound of formula (I), formula (I-a), formula (I-ai-iii), formula (I-b) and formula (I-bi-iii).

In some embodiments, the presently disclosed compounds can be “STING modulators.” As used herein, the term “STING modulator” refers to an agent that is capable of either activating or downregulating the STING pathway. A “STING modulator” can be an agonist or an antagonist.

As used herein, the term “agonist” and grammatical variations thereof refer to an agent, such as a small molecule or protein, that binds to a protein and causes, enhances, or augments (to a statistically significant degree) a particular biological effect of the protein. In certain embodiments, an agonist activates a receptor. The activation can be full, partial, or inverse. A full agonist has high efficacy, producing a full response while occupying a relatively low proportion of receptors. A partial agonist has lower efficacy than a full agonist. It produces sub-maximal activation even when occupying the total receptor population, therefore cannot produce the maximal response, irrespective of the concentration applied. An inverse agonist produces an effect opposite to that of an agonist, yet binds to the same receptor binding-site as an agonist. Agonists can be naturally occurring or artificially synthesized compounds.

For example, an agonist of a protein in the STING pathway is a compound that augments the natural activity of a protein in the STING pathway (either upstream or downstream). In particular, in some embodiments, the presently disclosed compounds activate STING-mediated signaling in human cells.

In some embodiments, the presently disclosed subject matter provides a “STING antagonist.” As used herein, the term “STING antagonist” refers to a presently disclosed compound that inhibits the STING pathway. In some embodiments, the STING antagonist interacts directly with the STING protein. In some embodiments, the STING antagonist interacts with a downstream component of the STING pathway, for example, cyclic GMP-AMP synthase (cGAS), TBK1, IRF3 or IFN-β. In some embodiments, the STING antagonist reduces the level or activity of one or more components of the STING pathway, e.g. STING, cyclic GMP-AMP synthase (cGAS), TBK1, IRF3 and/or IFN-β.

In some embodiments, the presently disclosed subject matter provides a method for treating a disease, disorder, or condition associated with STING, the method comprising administering a therapeutically effective amount of a compound of formula (I), formula (I-a), formula (I-ai-iii), formula (I-b) and formula (I-bi-iii) to a subject in need of treatment thereof.

In certain embodiments, the disease, disorder, or condition is selected from cancer, a bacterial infection, a viral infection, a fungal infection, a parasitic infection, an immune-mediated disorder, a central nervous system disease, a peripheral nervous system disease, a neurodegenerative disease, a mood disorder, a sleep disorder, a cerebrovascular disease, a peripheral artery disease, and a cardiovascular disease.

In particular embodiments, the disease, disorder, or condition is cancer. In more particular embodiments, the cancer is selected from colorectal cancer, aero-digestive squamous cancer, lung cancer, brain cancer, liver cancer, stomach cancer, sarcoma, leukemia, lymphoma, multiple myeloma, ovarian cancer, uterine cancer, breast cancer, melanoma, prostate cancer, bladder cancer, pancreatic carcinoma, and renal carcinoma.

In some embodiments, the presently disclosed method further comprises administering a second therapeutic agent. In certain embodiments, the second therapeutic agent is selected from an antiviral agent, an anti-inflammation agent, a chemotherapeutic agent, an anti-cancer vaccine, and hormonal therapy.

In particular embodiments, the second therapeutic agent is selected from a B7 costimulatory molecule, interleukin-2, interferon-g, GM-CSF, a CTLA-4 antagonist, an IDO inhibitor or IDO/TDO inhibitor, a PD-1 inhibitor, a PD-L1 inhibitor, an OX-40 ligand, a LAG3 inhibitor, a CD40 ligand, a 41BB/CD137 ligand, a CD27 ligand, Bacille Calmette-Guerin (BCG), liposomes, alum, Freund's complete or incomplete adjuvant, a TLR agonist, and a detoxified endotoxin.

In more particular embodiments, the CTLA-4 antagonist is ipilimumab or tremilimumab.

In more particular embodiments, the IDO inhibitor or IDO/TDO inhibitor is epacadostat or GDC-0919.

In more particular embodiments, the PD-1 inhibitor is selected from nivolumab, pembrolizumab, pidilizumab, AMP-224, and MDX-1106.

In more particular embodiments, the PD-L1 inhibitor is selected from durvalumab, avelumab and atezolizumab.

In more particular embodiments, the TLR agonist is selected from Poly I: C, MPL, LPS, bacterial flagellin, imiquimod, resiquimod, loxoribine and a CpG dinucleotide.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a compound described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the compounds described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of a compound described herein and at least one additional therapeutic agent can be varied so long as the beneficial effects of the combination of these agents are achieved. Accordingly, the phrase “in combination with” refers to the administration of a compound described herein and at least one additional therapeutic agent either simultaneously, sequentially, or a combination thereof. Therefore, a subject administered a combination of a compound described herein and at least one additional therapeutic agent can receive a compound and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the compound described herein and at least one additional therapeutic agent are administered simultaneously, they can be administered to the subject as separate pharmaceutical compositions, each comprising either a compound or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of a compound described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( SI )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### C. Pharmaceutical Compositions and Administration

In another aspect, the present disclosure provides a pharmaceutical composition including one compound described herein alone or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, trifluoroacetic acid (TFA), and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

In particular embodiments, the compound of formulae (I-III) is administered intranasally in a form selected from the group consisting of a nasal spray, a nasal drop, a powder, a granule, a cachet, a tablet, an aerosol, a paste, a cream, a gel, an ointment, a salve, a foam, a paste, a lotion, a cream, an oil suspension, an emulsion, a solution, a patch, and a stick.

As used herein, the term administrating via an “intranasal route” refers to administering by way of the nasal structures. It has been found that the presently disclosed small molecule compounds of formula (I) are much more effective at penetrating the brain and peripheral nervous system when administered intranasally.

As used herein, the term “peripheral nervous system” includes the part of the nervous system comprising the nerves and ganglia on the outside of the brain and spinal cord. The peripheral nervous system connects the central nervous system to the limbs and organs and acts as a communication relay between the brain and the extremities. The presently disclosed small molecule compounds of formula (I) can access the peripheral nervous system through the blood.

Intranasal administration generally allows the active agent to bypass first pass metabolism, thereby enhancing the bioavailability of the active agent. Such delivery can offer several advantages over other modes of drug delivery, including, but not limited to, increasing the onset of action, lowering the required dosage, enhancing the efficacy, and improving the safety profile of the active agent. For example, tablet dosage forms enter the bloodstream through the gastrointestinal tract, which subjects the drug to degradation from stomach acid, bile, digestive enzymes, and other first pass metabolism effects. As a result, tablet formulations often require higher doses and generally have a delayed onset of action. Nasal administration of a drug also can facilitate compliance, especially for pediatric patients, geriatric patients, patients suffering from a neurodegenerative disease, or other patients for which swallowing is difficult, e.g., patients suffering from nausea, such as patients undergoing chemotherapy, or patients with a swallowing disorder.

Intranasal (“i.n.” or “IN”) delivery of an agent to a subject can facilitate delivery of the agent to the brain and/or peripheral nervous system. Such administration is non-invasive and offers several advantages including avoidance of hepatic first pass clearance, rapid onset of action, frequent self-administration and easy dose adjustments. Small molecules have an added advantage of being absorbed paracellularly through the nasal epithelium after which, these molecules can then directly enter the CNS through the olfactory or the trigeminal nerve associated pathway and can be directly transported to the brain upon intranasal administration.

For intranasal delivery, in addition to the active ingredients, pharmaceutical compositions may contain suitable pharmaceutically acceptable carriers comprising excipients and auxiliaries which facilitate processing of the active compounds into preparations which can be used pharmaceutically. The agents of the disclosure may be formulated by methods known to those of skill in the art, and may include, for example, but not limited to, examples of solubilizing, diluting, or dispersing substances, such as saline, preservatives, such as benzyl alcohol, absorption promoters, and fluorocarbons. Optimized formulations for intranasal delivery may include addition of permeability enhancers (mucoadhesives, nanoparticles, and the like) as well as combined use with an intranasal drug delivery device (for example, one that provides controlled particle dispersion with particles aerosolized to target the upper nasal cavity).

In particular, polymer-based nanoparticles, including chitosan, maltodextrin, polyethylene glycol (PEG), polylactic acid (PLA), polylactic-co-glycolic acid (PLGA), and PAMAM dendrimer; gels, including poloxamer; and lipid-based formulations, including glycerol monocaprate (Capmul™), mixtures of mono-, di-, and triglycerides and mono- and di-fatty esters of PEG (Labrafil™), palmitate, glycerol monostearate, and phospholipids can be used to administer the presently disclosed compounds of formula (I) intranasally.

The presently disclosed compounds of formula (I) also can be administered intranasally via mucoadhesive agents. Mucoadhesion is commonly defined as the adhesion between two materials, at least one of which is a mucosal surface. More particularly, mucoadhesion is the interaction between a mucin surface and a synthetic or natural polymer. Mucoadhesive dosage forms can be designed to enable prolonged retention at the site of application, providing a controlled rate of drug release for improved therapeutic outcome. Application of dosage forms to mucosal surfaces may be of benefit to drug molecules not amenable to the oral route, such as those that undergo acid degradation or extensive first-pass metabolism. Mucoadhesive materials suitable for use with nasal administration of the presently disclosed compounds of formula (I) include, but are not limited to, soluble cellulose derivatives, such as hydroxypropyl methylcellulose (HPMC), hydroxypropyl cellulose (HPC), methylcellulose (MC), and carboxymethyl cellulose (CMC), and insoluble cellulose derivatives, such as ethylcellulose and microcrystalline cellulose (MCC), starch (e.g., Amioca®), polyacrylates, such as poly(acrylic acid) (e.g., Carbopol® 974P), functionalized mucoadhesive polymers, such as polycarbophil, hyaluronan, and amberlite resin, and chitosan (2-amino-2-deoxy-(1→4)-β-d-glucopyranan) formulations and derivatives thereof.

In some embodiments, the formulation also includes a permeability enhancer. As used herein, the term “permeability enhancer” refers to a substance that facilitates the delivery of a drug across mucosal tissue. The term encompasses chemical enhancers that, when applied to the mucosal tissue, render the tissue more permeable to the drug.

Permeability enhancers include, but are not limited to, dimethyl sulfoxide (DMSO), hydrogen peroxide (H₂O₂), propylene glycol, oleic acid, cetyl alcohol, benzalkonium chloride, sodium lauryl sulphate, isopropyl myristate, Tween 80, dimethyl formamide, dimethyl acetamide, sodium lauroylsarcosinate, sorbitan monolaurate, methylsulfonylmethane, Azone, terpenes, phosphatidylcholine dependent phospholipase C, triacyl glycerol hydrolase, acid phosphatase, phospholipase A2, concentrated saline solutions (e.g., PBS and NaCl), polysorbate 80, polysorbate 20, sodium dodecanoate (C12), sodium caprate (CIO) and/or sodium palmitate (CI 6), tert-butyl cyclohexanol (TBCH), and alpha-terpinol.

In some embodiments, the intranasal administration is accomplished via a ViaNase™ device (Kurve Technology, Inc.).

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Synthesis of Compounds of Formula (I)

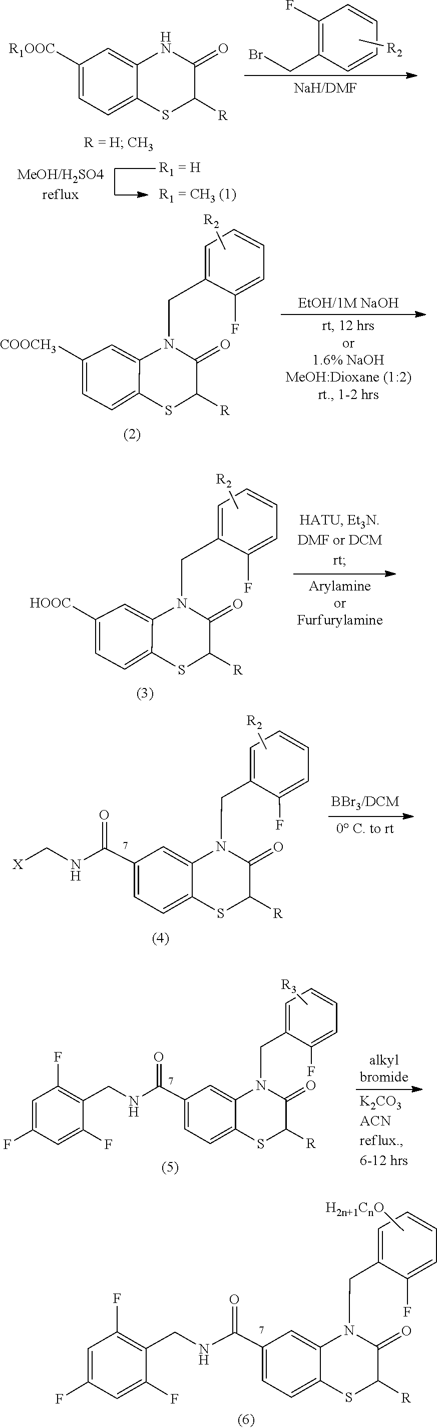

### Methylation (Synthesis of Compound 1):

To a stirred solution of a core molecule (1.0 eq.) in MeOH (10 mL) was added two or three drops of H₂SO₄ and the mixture was allowed to reflux for 3 hours. The reaction was monitored by TLC. After completion of the reaction, the reaction mixture was concentrated and then diluted with water and extracted with DCM, and the organic layers were washed with brine and dried over anhydrous MgSO₄. The organics were evaporated under reduced pressure to obtain the crude product which was used for next steps without further purification.

### Arylation (Synthesis of Compound 2):

To a stirred solution of a compound 1 (1.0 eq.) in DMF (10 mL) was added NaH (2.0 eq) followed by addition of substituted aryl bromide (1.2 eq.) and the mixture allowed to stir at room temperature for 0.5-1 hours. The reaction was monitored by TLC. After completion of the reaction, the reaction mixture was diluted with water, extracted with EtOAc, and the organic layers were washed with brine and dried over anhydrous MgSO₄. The organics were evaporated under reduced pressure to obtain the crude product which was used for next steps without further purification.

### Hydrolysis of Ester (Synthesis of Compound 3):

Method 1: To a stirred solution of compound 2 (1 mmol) in a mixture of MeOH and Dioxane (1:2; 9 mL) was added 1.6% NaOH (3 mL) at room temperature. The reaction was allowed to stir for 2 hours. Upon completion of the reaction, water was added. The aqueous solution was acidified with 7% HCl and then extracted with EtOAc. The organic layer was dried and evaporated to dryness to obtain the crude product which was used for next steps without further purification.

Method 2: To a stirred solution of compound 2 (1 mmol) in EtOH (20 mL) was added 1M NaOH (10 mL) at room temperature. The reaction was allowed to stir for 2 hours. Upon completion of the reaction, water was added. The aqueous solution was acidified with 1M HCl and then extracted with EtOAc. The organic layer was dried and evaporated to dryness to obtain the crude product which was used for next steps without further purification.

### Amidation (Synthesis of Compound 4):

To a stirred solution of a carboxylic acid 3 (1 eq.) in a suitable solvent, such as DCM or DMF (10 mL) was added amine (1.3 eq.) and a coupling Reagent i-e HATU (2.0 cq.) followed by addition of TEA (2.0 eq.) drop wise to the solution and the mixture allowed to stir at room temperature for 2-3 hours. When TLC showed completion of the reaction, the reaction mixture was diluted with ice-cooled water and extracted with EtOAc. The combined organic layers were washed with water and then dried over anhydrous MgSO₄. The solvent was evaporated under reduced pressure to obtain the crude material which was purified by column chromatography using mixtures of EtOAc in heptane as eluent to afford a compound (70-80% yield). Some of the compounds on addition of the crude product into ice-cooled water precipitated out. Collect the precipitates using filtration technique. Washed it with water 3 times and then purified the product using column chromatography.

### Deprotection Reaction (Synthesis of 5):

The corresponding compound 4 (1 eq.) was dissolved in dry dichloromethane using a Pheat-dried three neck round bottom flask equipped with a magnetic stirrer. This solution was cooled down using ice and acetone, to about −20° C.; then a solution of boron tribromide (1M in CH₂Cl₂; 3 eq.) was added dropwise. After adding the deprotecting agent, the ice bath was withdrawn to allow the reaction mixture to warm up to room temperature and it was stirred for 1-4 hour. The reaction was quenched by adding ice-cooled water (60 mL; very slowly), and the product was extracted with dichloromethane (20 mL). The combined organic phases were dried using sodium sulfate, and the solvent was evaporated under vacuum. Finally, the product was purified by column chromatography using a mixture of ethyl acetate/heptane to afford the corresponding hydroxylated compound.

### Alkylation (Synthesis of 6):

To a stirred solution of a compound 5 (1 eq.) in ACN (10 mL) was added K₂CO₃ (5.0 eq.) followed by addition of alkyl bromide (1.2 eq.) and the mixture allowed to reflux for 2-3 hour. When TLC showed completion of the reaction, the reaction mixture was diluted with water and extracted with EtOAc. The combined organic layers were washed with water and then dried over anhydrous MgSO₄. The solvent was evaporated under reduced pressure to obtain the crude material, which was purified by column chromatography using mixtures of EtOAc in heptane as eluent to afford a compound.

Representative compounds of formula (I) are provided in Table 1.

TABLE 1
Representative Compounds of Formula (I)

| Compound | Structure |
|---|---|
| INI-3067 | 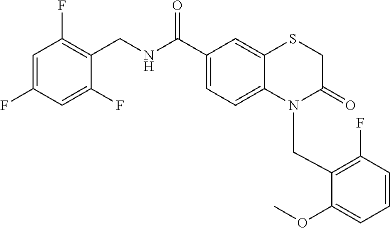 [SMILES: COc1cccc(F)c1CN1C(=O)CSc2cc(C(=O)NCc3c(F)cc(F)cc3F)ccc21] |
| INI-3069 | 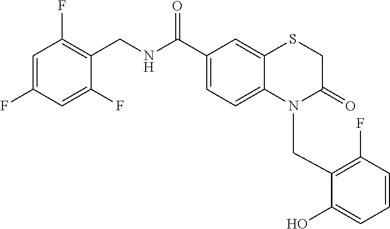 [SMILES: O=C(NCc1c(F)cc(F)cc1F)c1ccc2c(c1)SCC(=O)N2Cc1c(O)cccc1F] |
| INI-3070 | 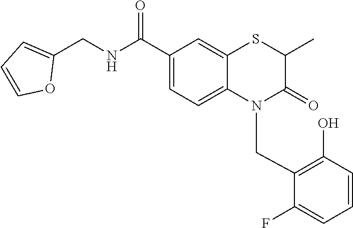 [SMILES: CC1Sc2cc(C(=O)NCc3ccco3)ccc2N(Cc2c(O)cccc2F)C1=O] |
| INI-3071 | 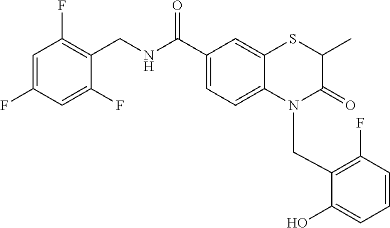 [SMILES: CC1Sc2cc(C(=O)NCc3c(F)cc(F)cc3F)ccc2N(Cc2c(O)cccc2F)C1=O] |
| INI-3077 | 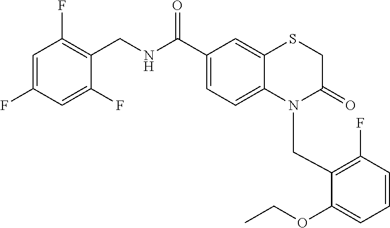 [SMILES: CCOc1cccc(F)c1CN1C(=O)CSc2cc(C(=O)NCc3c(F)cc(F)cc3F)ccc21] |
| INI3078 | 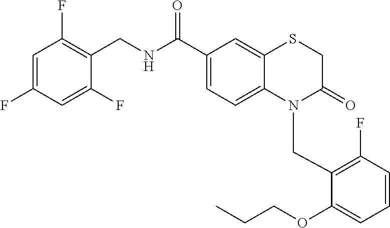 [SMILES: CCCOc1cccc(F)c1CN1C(=O)CSc2cc(C(=O)NCc3c(F)cc(F)cc3F)ccc21] |
| INI-3079 | 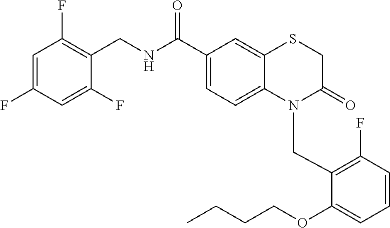 [SMILES: CCCCOc1cccc(F)c1CN1C(=O)CSc2cc(C(=O)NCc3c(F)cc(F)cc3F)ccc21] |
| INI-3080 | 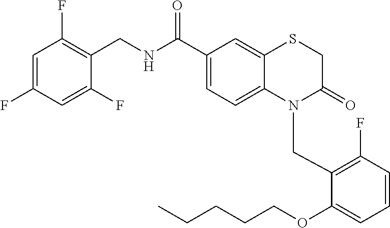 [SMILES: CCCCCOc1cccc(F)c1CN1C(=O)CSc2cc(C(=O)NCc3c(F)cc(F)cc3F)ccc21] |
| INI-3081 | 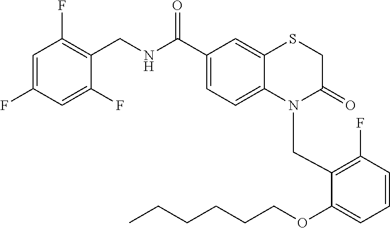 [SMILES: CCCCCCOc1cccc(F)c1CN1C(=O)CSc2cc(C(=O)NCc3c(F)cc(F)cc3F)ccc21] |
| INI-3110 | 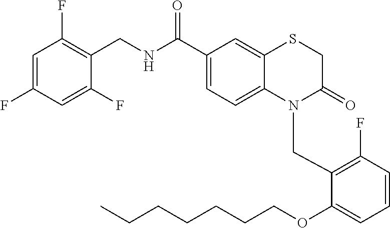 [SMILES: CCCCCCCOc1cccc(F)c1CN1C(=O)CSc2cc(C(=O)NCc3c(F)cc(F)cc3F)ccc21] |
| INI-3111 | 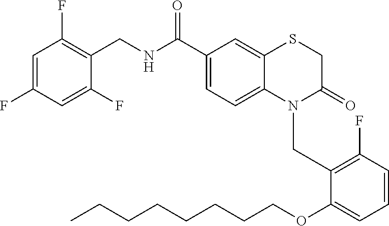 [SMILES: CCCCCCCCOc1cccc(F)c1CN1C(=O)CSc2cc(C(=O)NCc3c(F)cc(F)cc3F)ccc21] |
| INI-3112 | 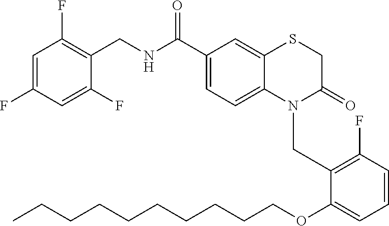 [SMILES: CCCCCCCCCCOc1cccc(F)c1CN1C(=O)CSc2cc(C(=O)NCc3c(F)cc(F)cc3F)ccc21] |
| INI-3113 | 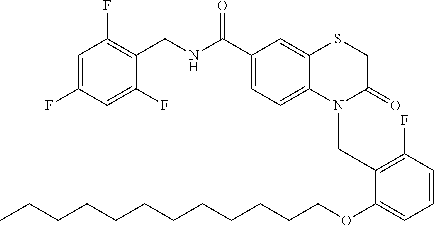 [SMILES: CCCCCCCCCCCCOc1cccc(F)c1CN1C(=O)CSc2cc(C(=O)NCc3c(F)cc(F)cc3F)ccc21] |
| INI-3114 | 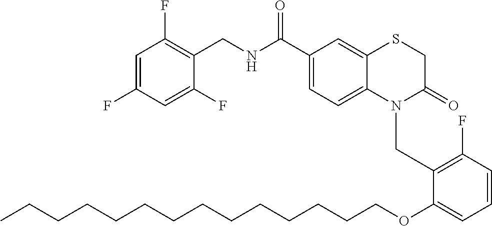 [SMILES: CCCCCCCCCCCCCCOc1cccc(F)c1CN1C(=O)CSc2cc(C(=O)NCc3c(F)cc(F)cc3F)ccc21] |
| INI-3115 | 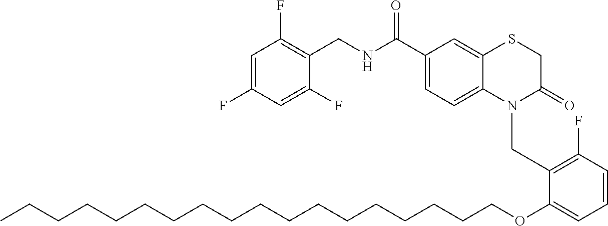 [SMILES: CCCCCCCCCCCCCCCCCCOc1cccc(F)c1CN1C(=O)CSc2cc(C(=O)NCc3c(F)cc(F)cc3F)ccc21] |
| INI-3075 | 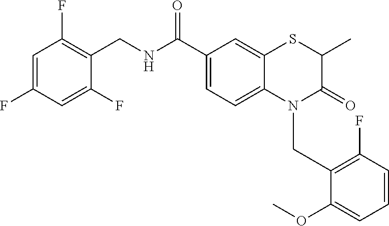 [SMILES: COc1cccc(F)c1CN1C(=O)C(C)Sc2cc(C(=O)NCc3c(F)cc(F)cc3F)ccc21] |
| INI-3105 | 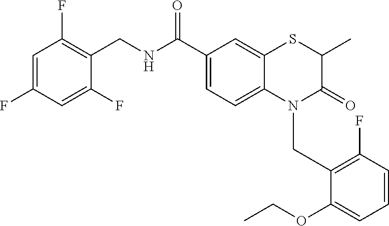 [SMILES: CCOc1cccc(F)c1CN1C(=O)C(C)Sc2cc(C(=O)NCc3c(F)cc(F)cc3F)ccc21] |
| INI-3106 | 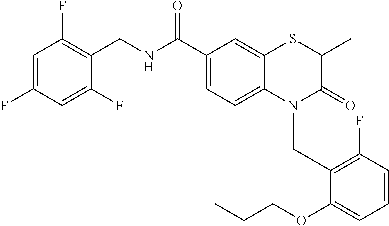 [SMILES: CCCOc1cccc(F)c1CN1C(=O)C(C)Sc2cc(C(=O)NCc3c(F)cc(F)cc3F)ccc21] |
| INI-3107 | 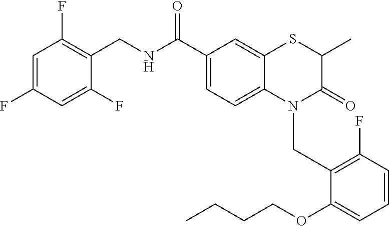 [SMILES: CCCCOc1cccc(F)c1CN1C(=O)C(C)Sc2cc(C(=O)NCc3c(F)cc(F)cc3F)ccc21] |
| INI-3108 | 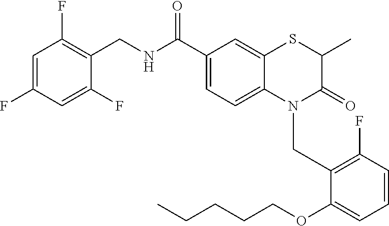 [SMILES: CCCCCOc1cccc(F)c1CN1C(=O)C(C)Sc2cc(C(=O)NCc3c(F)cc(F)cc3F)ccc21] |
| INI-3109 | 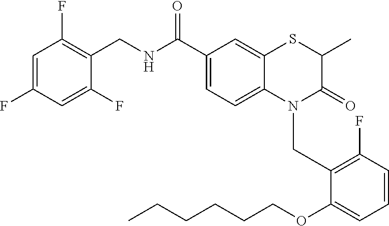 [SMILES: CCCCCCOc1cccc(F)c1CN1C(=O)C(C)Sc2cc(C(=O)NCc3c(F)cc(F)cc3F)ccc21] |
| INI-3074 | 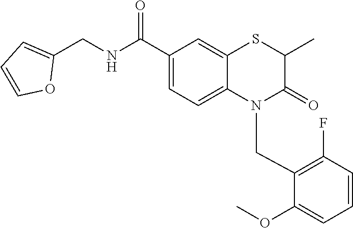 [SMILES: COc1cccc(F)c1CN1C(=O)C(C)Sc2cc(C(=O)NCc3ccco3)ccc21] |
| INI-3076 | 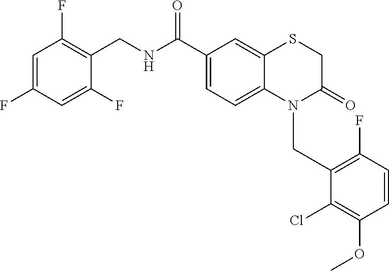 [SMILES: COc1ccc(F)c(CN2C(=O)CSc3cc(C(=O)NCc4c(F)cc(F)cc4F)ccc32)c1Cl] |
| INI-3082 | 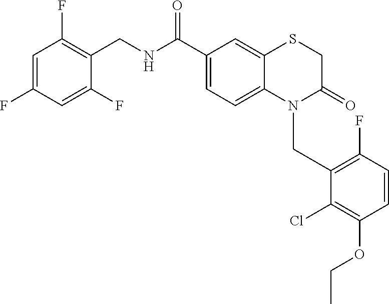 [SMILES: CCOc1ccc(F)c(CN2C(=O)CSc3cc(C(=O)NCc4c(F)cc(F)cc4F)ccc32)c1Cl] |
| INI-3083 | 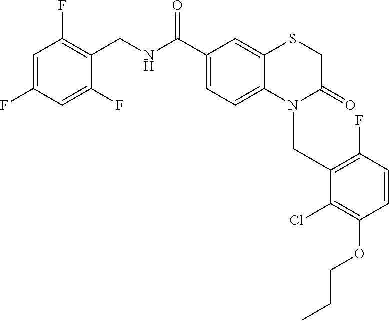 [SMILES: CCCOc1ccc(F)c(CN2C(=O)CSc3cc(C(=O)NCc4c(F)cc(F)cc4F)ccc32)c1Cl] |
| INI-3084 | 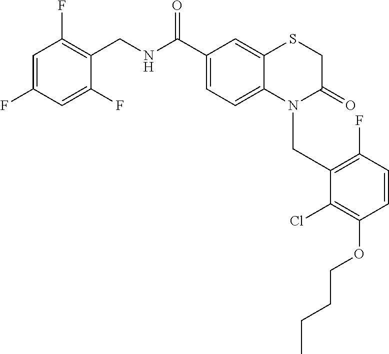 [SMILES: CCCCOc1ccc(F)c(CN2C(=O)CSc3cc(C(=O)NCc4c(F)cc(F)cc4F)ccc32)c1Cl] |
| INI-3085 | 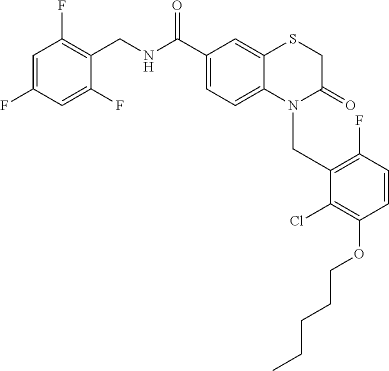 [SMILES: CCCCCOc1ccc(F)c(CN2C(=O)CSc3cc(C(=O)NCc4c(F)cc(F)cc4F)ccc32)c1Cl] |
| INI-3086 | 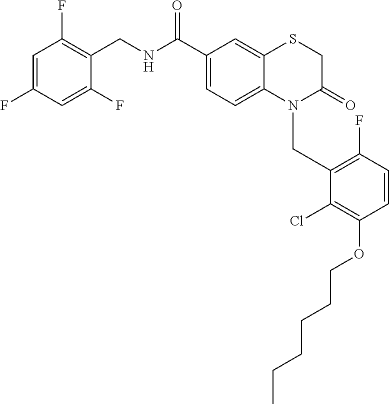 [SMILES: CCCCCCOc1ccc(F)c(CN2C(=O)CSc3cc(C(=O)NCc4c(F)cc(F)cc4F)ccc32)c1Cl] |
| INI-3087 (prior art) | 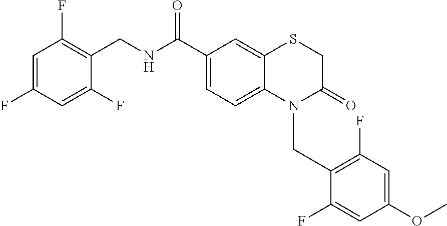 [SMILES: COc1cc(F)c(CN2C(=O)CSc3cc(C(=O)NCc4c(F)cc(F)cc4F)ccc32)c(F)c1] |
| INI-3088 | 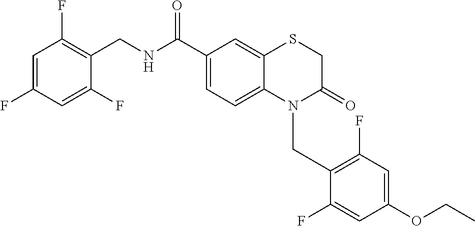 [SMILES: CCOc1cc(F)c(CN2C(=O)CSc3cc(C(=O)NCc4c(F)cc(F)cc4F)ccc32)c(F)c1] |
| INI-3089 | 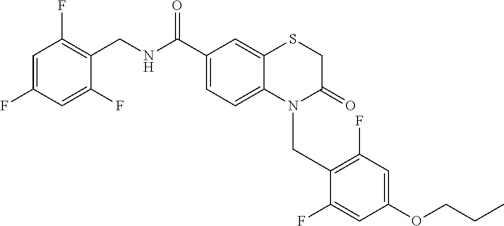 [SMILES: CCCOc1cc(F)c(CN2C(=O)CSc3cc(C(=O)NCc4c(F)cc(F)cc4F)ccc32)c(F)c1] |
| INI-3090 | 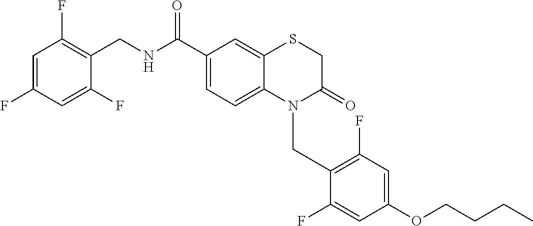 [SMILES: CCCCOc1cc(F)c(CN2C(=O)CSc3cc(C(=O)NCc4c(F)cc(F)cc4F)ccc32)c(F)c1] |
| INI-3091 | 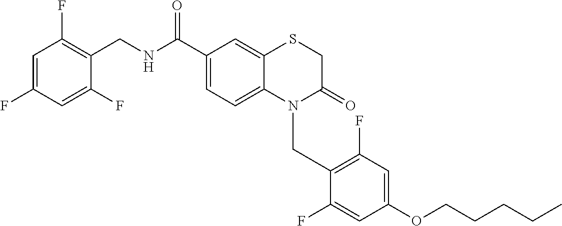 [SMILES: CCCCCOc1cc(F)c(CN2C(=O)CSc3cc(C(=O)NCc4c(F)cc(F)cc4F)ccc32)c(F)c1] |
| INI-3092 | 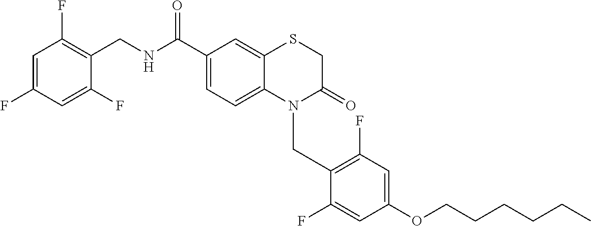 [SMILES: CCCCCCOc1cc(F)c(CN2C(=O)CSc3cc(C(=O)NCc4c(F)cc(F)cc4F)ccc32)c(F)c1] |
| INI-3095 | 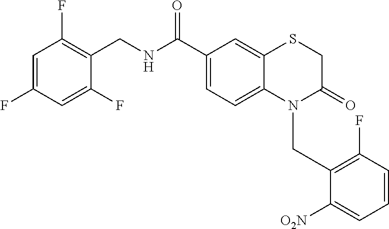 [SMILES: O=C(NCc1c(F)cc(F)cc1F)c1ccc2c(c1)SCC(=O)N2Cc1c(F)cccc1[N+](=O)[O-]] |
| INI-3093 | 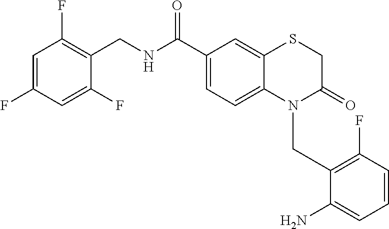 [SMILES: Nc1cccc(F)c1CN1C(=O)CSc2cc(C(=O)NCc3c(F)cc(F)cc3F)ccc21] |
| INI-3103 | 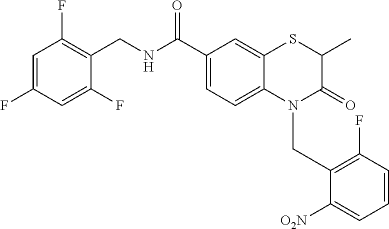 [SMILES: CC1Sc2cc(C(=O)NCc3c(F)cc(F)cc3F)ccc2N(Cc2c(F)cccc2[N+](=O)[O-])C1=O] |
| INI-3104 | 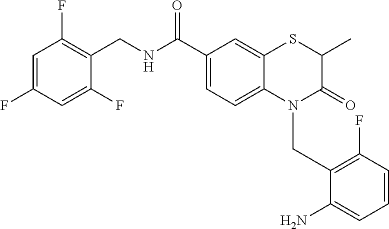 [SMILES: CC1Sc2cc(C(=O)NCc3c(F)cc(F)cc3F)ccc2N(Cc2c(N)cccc2F)C1=O] |
| INI-3094 | 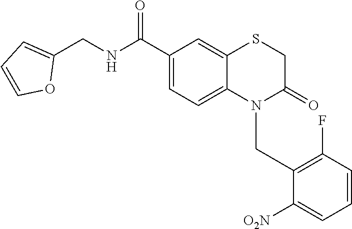 [SMILES: O=C(NCc1ccco1)c1ccc2c(c1)SCC(=O)N2Cc1c(F)cccc1[N+](=O)[O-]] |
| INI-3096 | 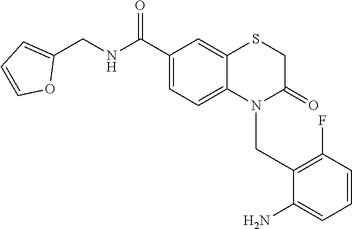 [SMILES: Nc1cccc(F)c1CN1C(=O)CSc2cc(C(=O)NCc3ccco3)ccc21] |

### Example 2

### THF Luciferase Activity

STING molecules were screened to determine human STING pathway specificity. THF-ISRE, THF-ISRE STING knock-out (KO), and THF-ISRE TRIF/MAVS KO cell lines contain an inducible luciferase reporter construct that will signal upon IRF3 activation. THF-ISRE STING KO cell line only has the TRIF/MAVS pathway to activate IRF3. THF-ISRE TRIF/MAVS KO cell line uses only the STING pathway for IRF3 activation. Luciferase activity was determined by measuring luminescence.

The three cell lines were maintained in T-75 flasks with DMEM/High Glucose (Cytiva™, Logan, UT), Pen/Strep/Glutamine (Hyclone™, Logan, UT), and 10% heat inactivated FBS (Hyclone™, Logan, UT).

For primary screening, THF-ISRE cells were plated at a density of 15,000 cells/well in opaque flat bottom 96-well tissue culture plates. Cells incubated for 36 hours and treated with DMSO (Sigma™, St. Louis, MO) formulated compounds. Before treatment, cell media was changed to 2% FBS in DMEM with antibiotics. Compounds were serially diluted 2-fold, starting at 50 μM and ending at 0.391 μM. Luciferase activity was measured after 18 hours by addition of Steady-Glo Luciferase Assay System (Promega™, Madison, WI) at a 1:1 ratio and luminescence read by plate reader. The compounds that demonstrated luciferase activity went through a secondary screening.

The secondary screening used the same procedure as above with THF-ISRE STING KO and THF-ISRE TRIF/MAVS KO cells. If the compound was human STING specific, only THF-ISRE TRIF/MAVS KO cells would have luciferase activity.

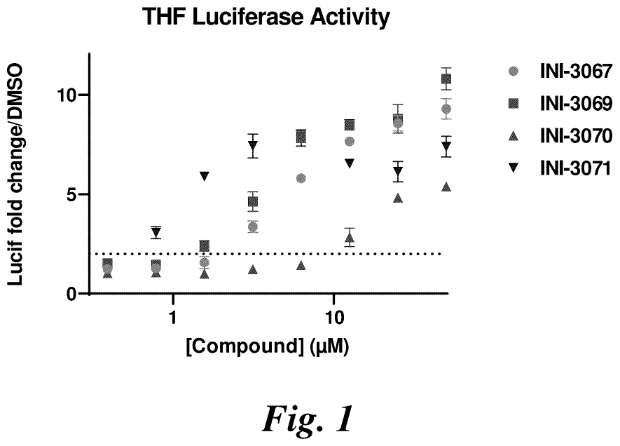

An initial screen demonstrated that representative compounds of formula (I) INI-3067, INI-3069, INI-3070, and INI-3071 exhibited positive luciferase activity. Referring now to FIG. 1, a secondary screen in THE TRIF/MAVS KO cells demonstrated that INI-3067, INI-3069, INI-3070, and INI-3071 use the STING pathway for luciferase activity.

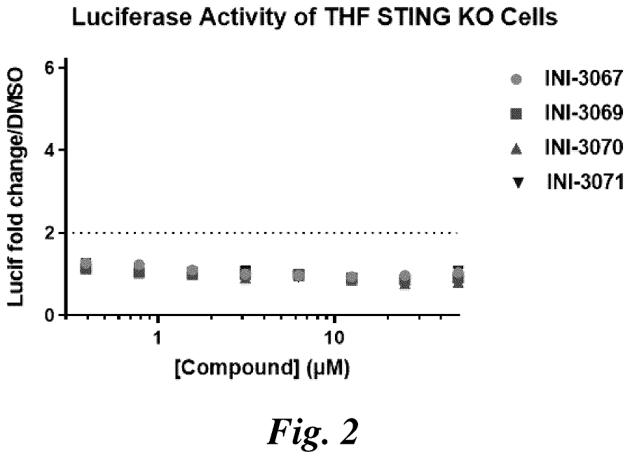

FIG. 2 shows the THF luciferase activity of compounds INI-3077, INI-3078, INI-3079, INI-3080, INI-3067, and INI-3069.

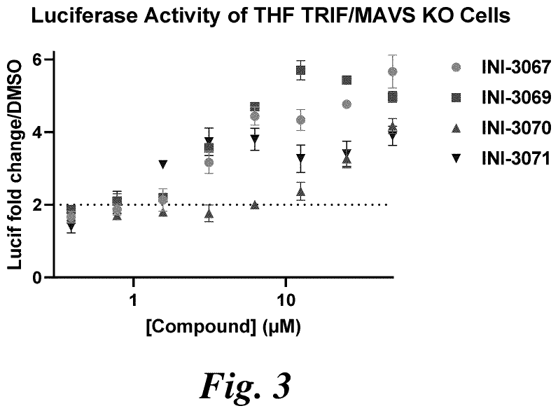

FIG. 3 shows the THF STING only luciferase activity of compounds INI-3077, INI-3078, INI-3079, INI-3080, INI-3081, and INI-3067.

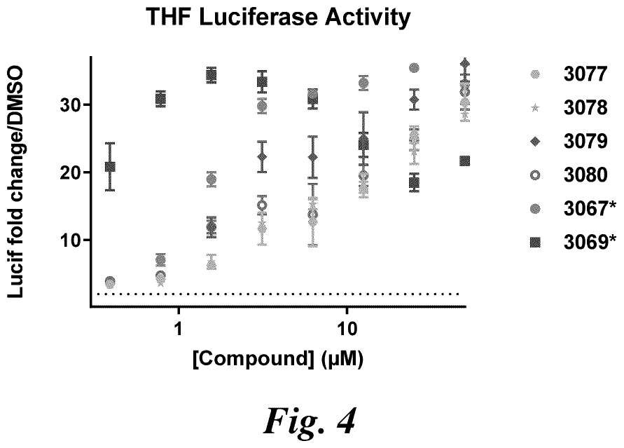

FIG. 4 shows the THF luciferase activity of compounds INI-3067, INI-3111, INI-3112, INI-3069, and INI-3110.

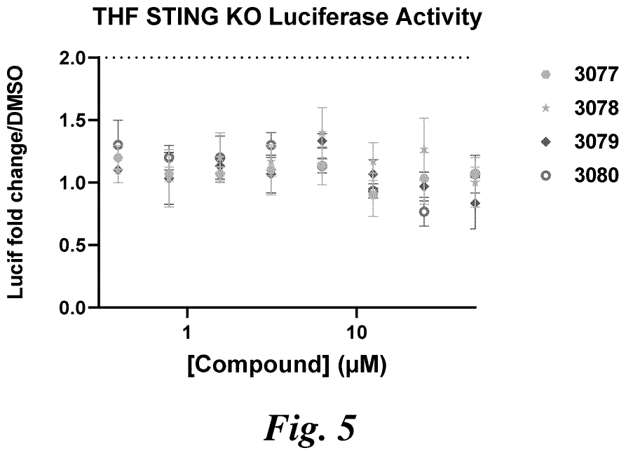

FIG. 5 shows the THF luciferase activity of compounds INI-3071, INI-3105, INI-3106, INI-3107, INI-3108, and INI-3109.

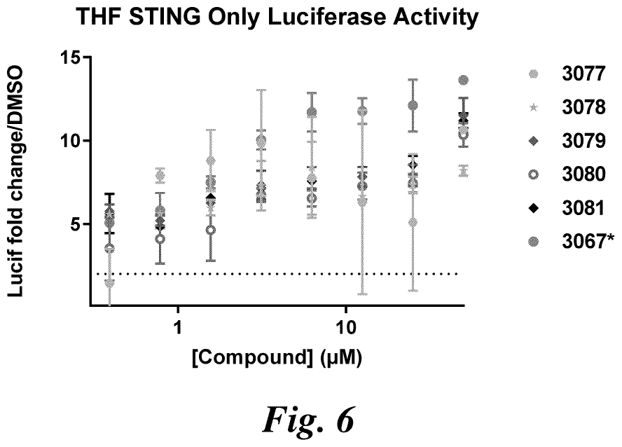

FIG. 6 shows the THF luciferase activity of compounds INI-3070, INI-3074, INI-3071, and INI-3075.

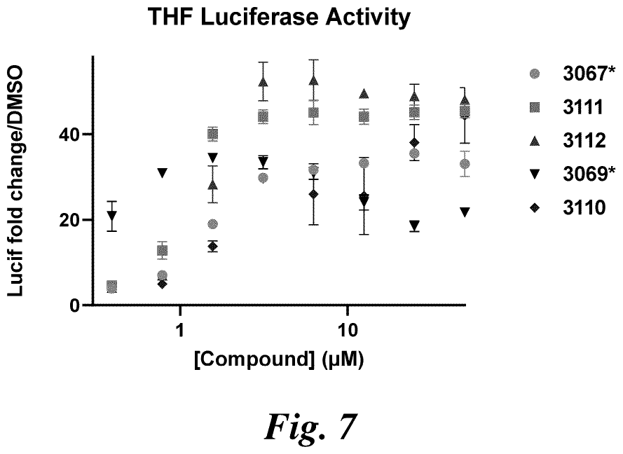

FIG. 7 shows the THF luciferase activity of compounds INI-3076, INI-3082, INI-3083, INI-3084, INI-3085, INI-3086, and INI-3069.

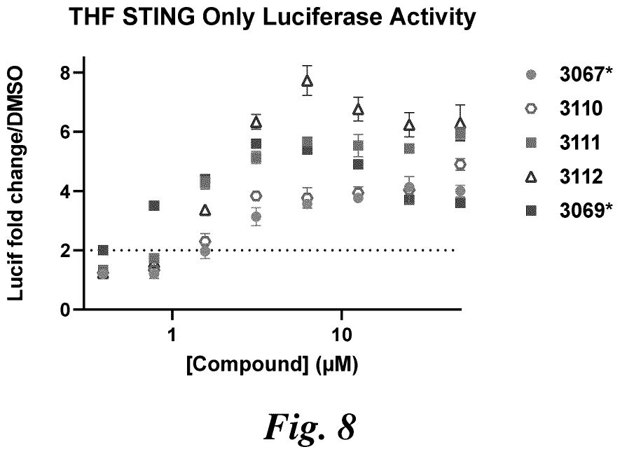

FIG. 8 shows the THF STING luciferase activity of compounds INI-3076, INI-3082, INI-3083, INI-3084, INI-3085, and INI-3086.

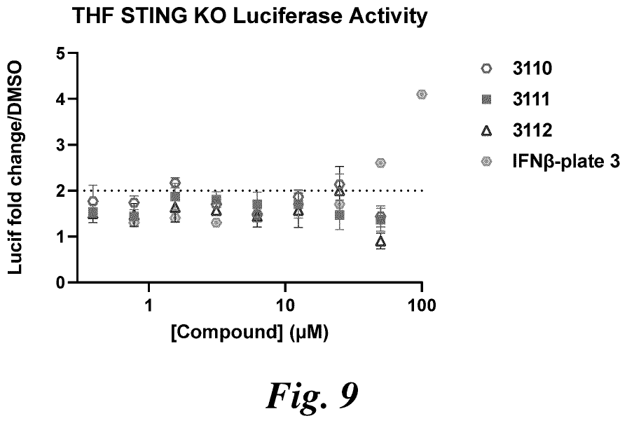

FIG. 9 shows the THF luciferase activity of compounds INI-3087, INI-3088, INI-3089, INI-3090, INI-3091, INI-3092, and INI-3069.

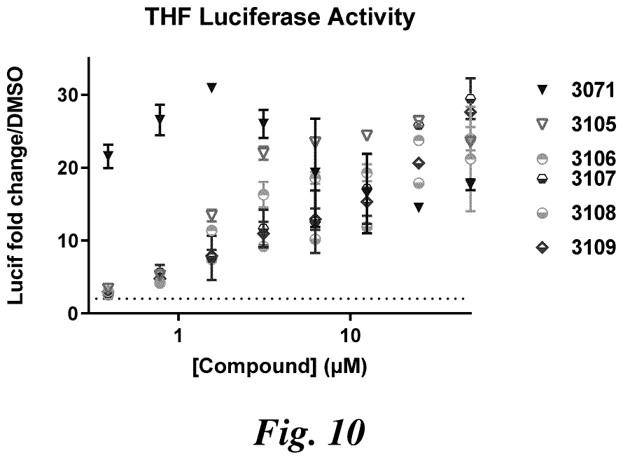

FIG. 10 shows the THF STING only luciferase activity of compounds INI-3087, INI-3088, INI-3089, INI-3090, INI-3091, and INI-3092.

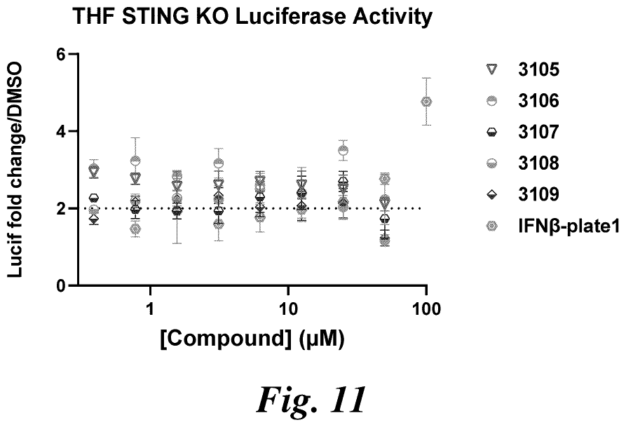

FIG. 11 shows the THF luciferase activity of compounds INI-3093, INI-3094, INI-3095, INI-3096, INI-3103, INI-3104, and INI-3069.

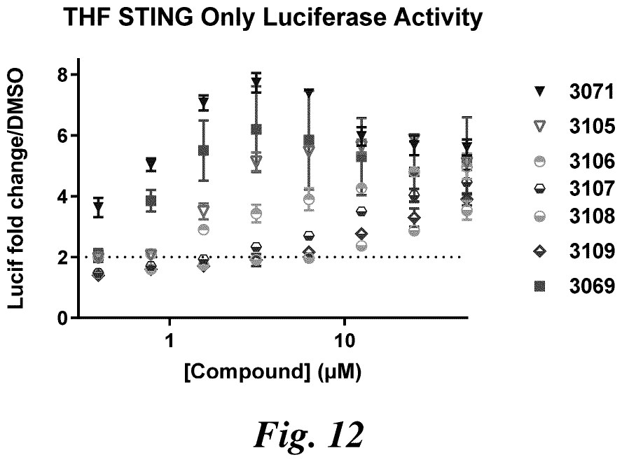

FIG. 12 shows the THF STING only luciferase activity of compounds INI-3093, INI-3094, INI-3095, INI-3096, INI-3103, and INI-3104.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents form part of the common general knowledge in the art.
D. C. Pryde, S. Middya, M. Banerjee, R. Shrivastava, S. Basu, R. Ghosh, D. B. Yadav, A. Surya, The discovery of potent small molecule activators of human STING, Eur. J. Med. Chem., 209, 112869 (2021).
M. Banerjee, S. Middya, R. Shrivastava, S. Basu, R. Ghosh, D. C. Pryde, et al., G10 is a direct activator of human STING, PLOS ONE 15 (9), e0237743 (2020).
U.S. Patent Application Publication No. 2020/0138827 A1 for SMALL MOLECULE MODULATORS OF HUMAN STING, to Banerjee et al., published May 7, 2020.
U.S. Patent Application No. 2017/0146519 A1 for STING AGONISTS AND METHODS OF SELECTED STING AGONISTS, to DeFilippis et al., published May 25, 2017.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A compound of formula (I): 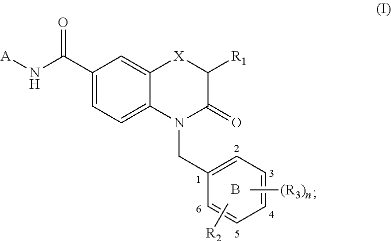
wherein:
n is 1 or 2;
X is selected from S, O, NRₓ, and CRₓ₁Rₓ₂, wherein Rₓ, Rₓ₁, and Rₓ₂ are each independently selected from H and C₁-C₄ alkyl;
A is: 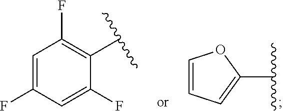 [SMILES: *c1c(F)cc(F)cc1F ; *c1ccco1]
R₁ is H or C₁-C₄ alkyl;
R₂ is selected from —OR₄, —NR₅R₆, and —NO₂, wherein R₄ is H or C₁-C₂₀ alkyl and R₅ and R₆ are each independently H or C₁-C₄ alkyl;
each R₃ can be the same or different and are each halogen;
wherein: (i) if X is S, A is 2,4,6-trifluorophenyl, n is 2, and R₃ is F in the 2 and 6 position of ring B, then R₄ cannot be H or C₁ alkyl; or (ii) if X is S, A is 2,4,6-trifluorophenyl, n is 2, and R₃ is F in the 2 position of ring B, then R₄ cannot be C₁ alkyl; and
pharmaceutically acceptable salts thereof.

2. The compound of claim 1, wherein the compound of formula (I) is a compound of formula (I′):

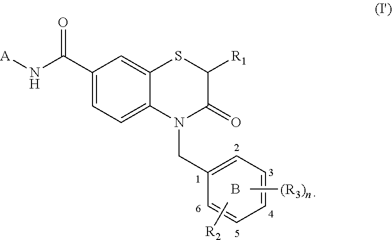

3. The compound of claim 2, wherein the compound of formula (I′) is a compound of formula (Ia) or formula (Ib):

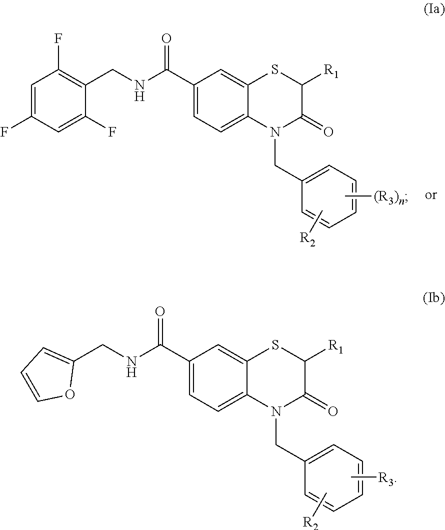

4. The compound of claim 3, wherein the compound of formula (Ia) is a compound selected from a compound of formula (Ia-i), formula (Ia-ii), and formula (Ia-iii):

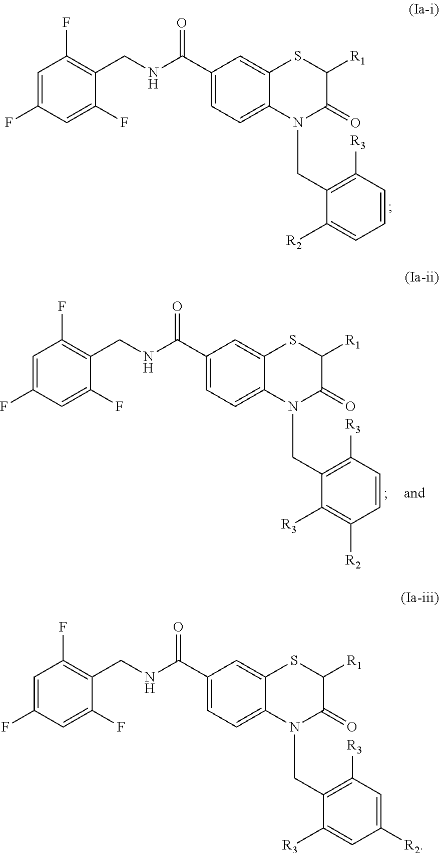

5. The compound of claim 4, wherein R₂ is —OR₄.

6. The compound of claim 5, wherein R₁ is H.

7. The compound of claim 6, wherein the compound of formula (Ia-i) is selected from:

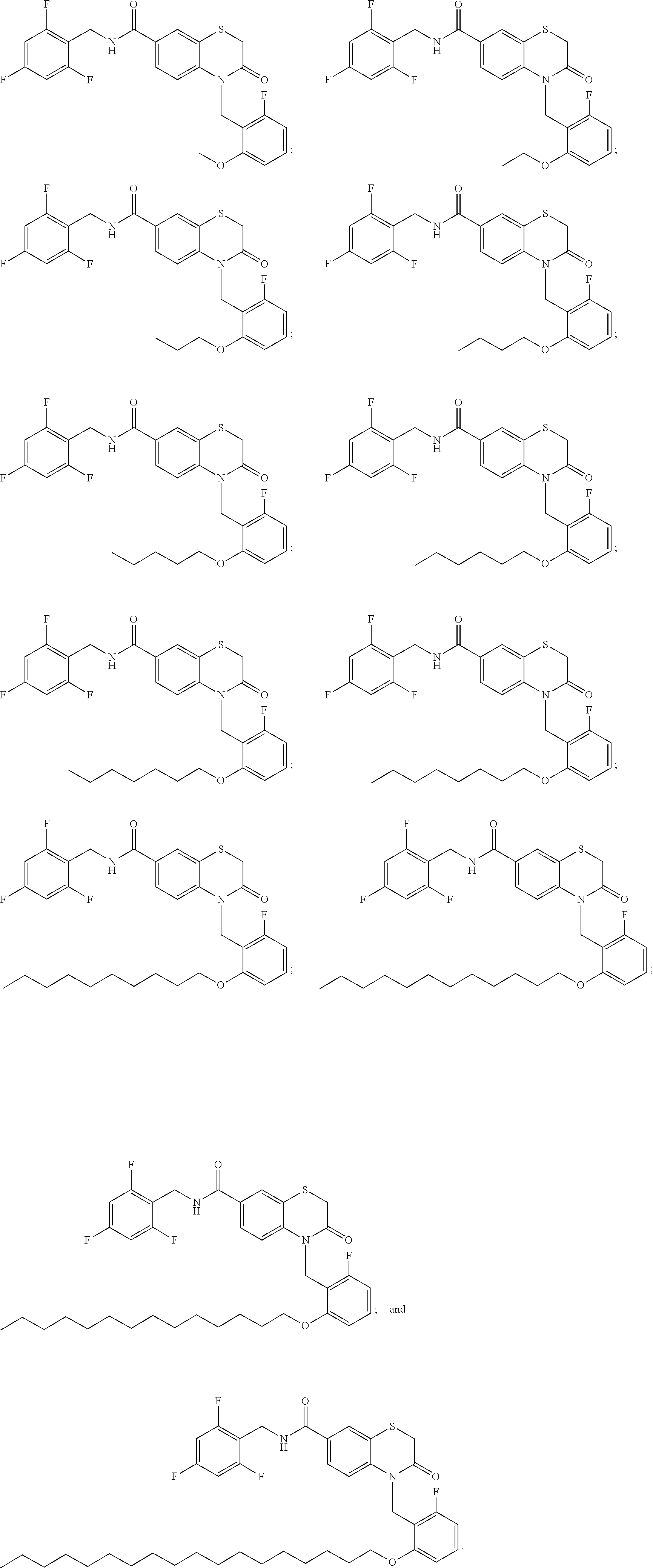

8. The compound of claim 5, wherein R₁ is C₁-C₄ alkyl.

9. The compound of claim 8, wherein the compound of formula (Ia-i) is selected from:

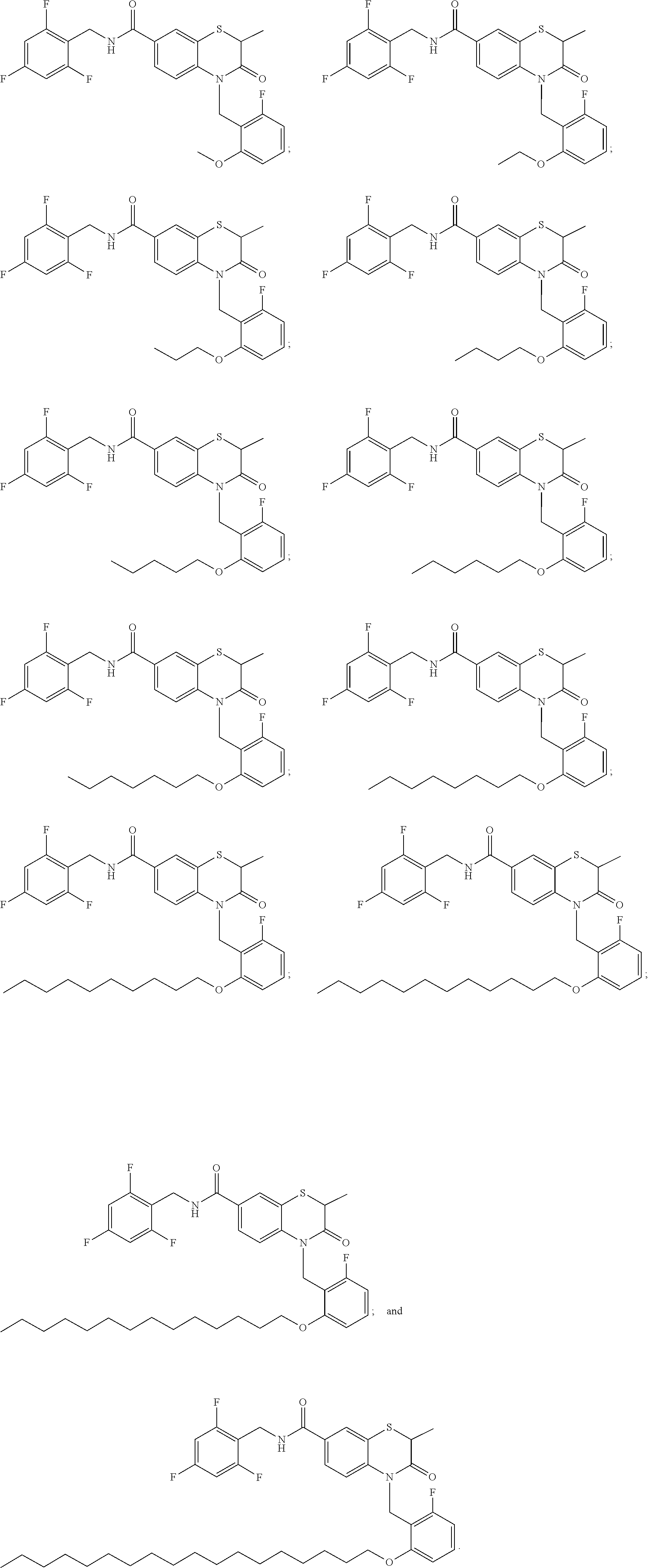

10-12. (canceled)

13. The compound of claim 6, wherein the compound of formula (Ia-ii) is selected from:

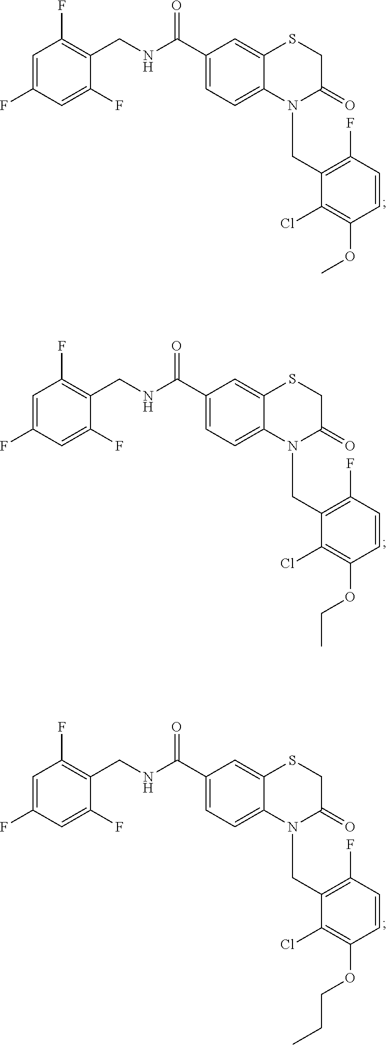 [SMILES: COc1ccc(F)c(CN2C(=O)CSc3cc(C(=O)NCc4c(F)cc(F)cc4F)ccc32)c1Cl ; CCOc1ccc(F)c(CN2C(=O)CSc3cc(C(=O)NCc4c(F)cc(F)cc4F)ccc32)c1Cl ; CCCOc1ccc(F)c(CN2C(=O)CSc3cc(C(=O)NCc4c(F)cc(F)cc4F)ccc32)c1Cl]

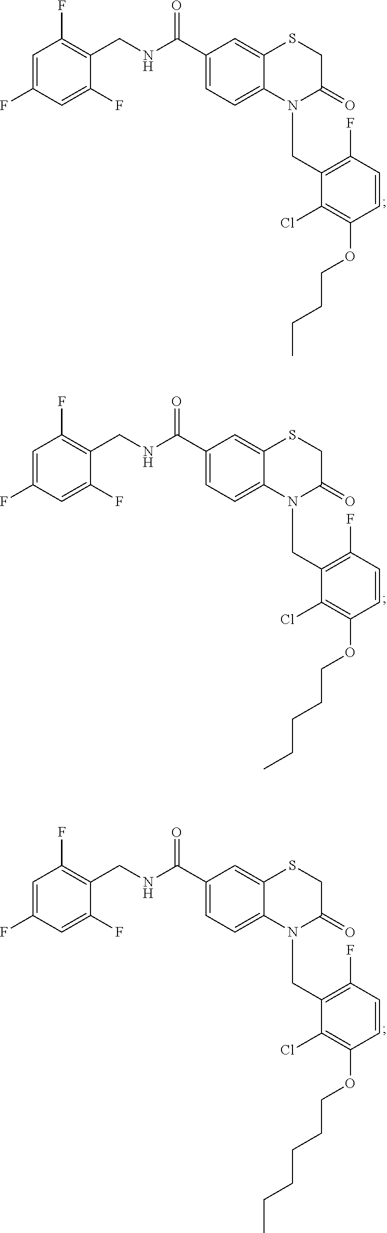 [SMILES: CCCCOc1ccc(F)c(CN2C(=O)CSc3cc(C(=O)NCc4c(F)cc(F)cc4F)ccc32)c1Cl ; CCCCCOc1ccc(F)c(CN2C(=O)CSc3cc(C(=O)NCc4c(F)cc(F)cc4F)ccc32)c1Cl ; CCCCCCOc1ccc(F)c(CN2C(=O)CSc3cc(C(=O)NCc4c(F)cc(F)cc4F)ccc32)c1Cl]

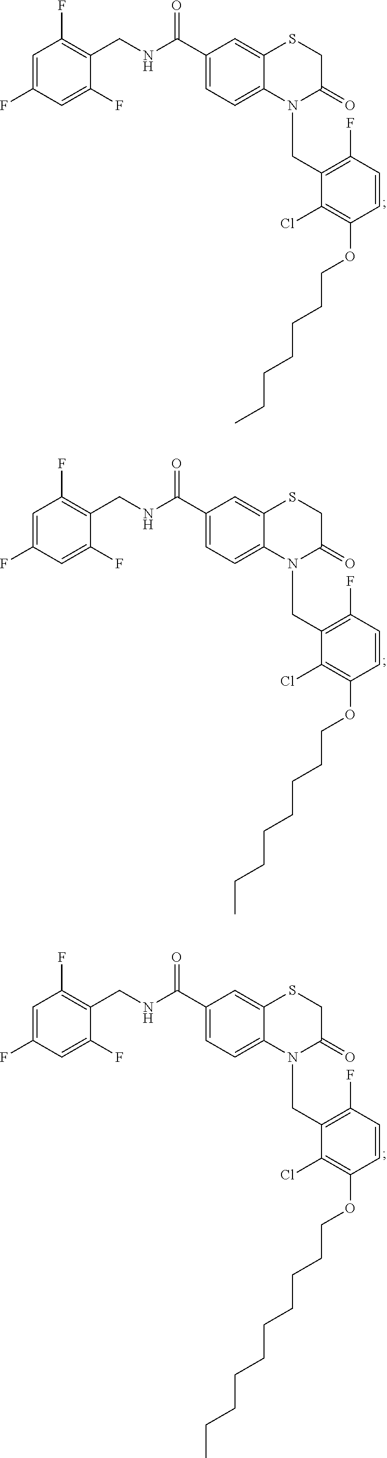 [SMILES: CCCCCCCOc1ccc(F)c(CN2C(=O)CSc3cc(C(=O)NCc4c(F)cc(F)cc4F)ccc32)c1Cl ; CCCCCCCCOc1ccc(F)c(CN2C(=O)CSc3cc(C(=O)NCc4c(F)cc(F)cc4F)ccc32)c1Cl ; CCCCCCCCCCOc1ccc(F)c(CN2C(=O)CSc3cc(C(=O)NCc4c(F)cc(F)cc4F)ccc32)c1Cl]

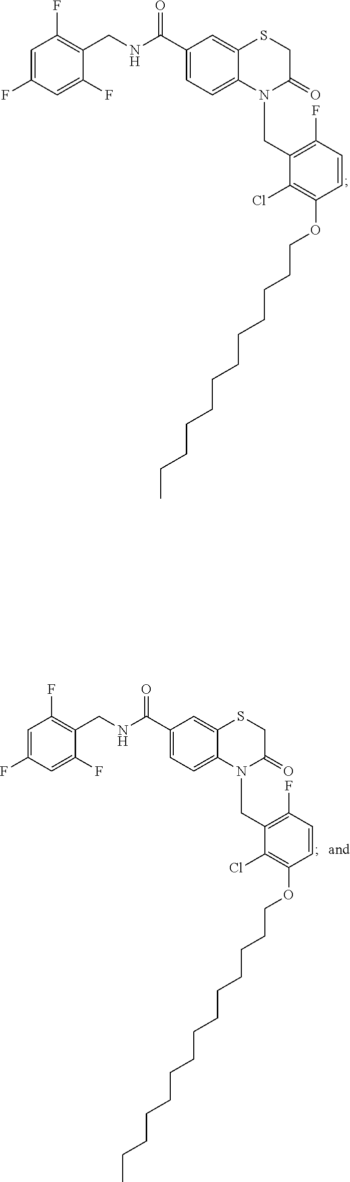 [SMILES: CCCCCCCCCCCCOc1ccc(F)c(CN2C(=O)CSc3cc(C(=O)NCc4c(F)cc(F)cc4F)ccc32)c1Cl ; CCCCCCCCCCCCCCOc1ccc(F)c(CN2C(=O)CSc3cc(C(=O)NCc4c(F)cc(F)cc4F)ccc32)c1Cl]

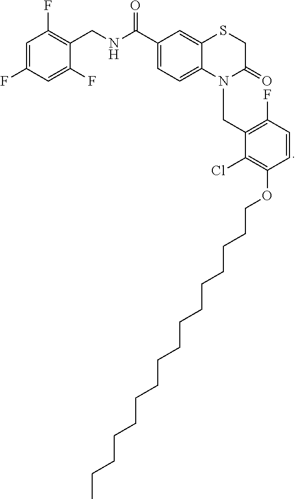 [SMILES: CCCCCCCCCCCCCCCCOc1ccc(F)c(CN2C(=O)CSc3cc(C(=O)NCc4c(F)cc(F)cc4F)ccc32)c1Cl]

14. (canceled)

15. The compound of claim 8, wherein the compound of formula (Ia-ii) is selected from:

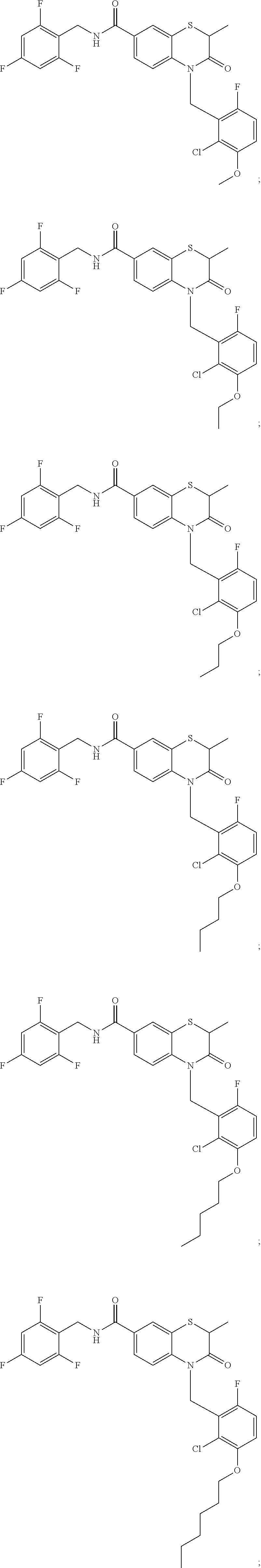 [SMILES: COc1ccc(F)c(CN2C(=O)C(C)Sc3cc(C(=O)NCc4c(F)cc(F)cc4F)ccc32)c1Cl ; CCOc1ccc(F)c(CN2C(=O)C(C)Sc3cc(C(=O)NCc4c(F)cc(F)cc4F)ccc32)c1Cl ; CCCOc1ccc(F)c(CN2C(=O)C(C)Sc3cc(C(=O)NCc4c(F)cc(F)cc4F)ccc32)c1Cl ; CCCCOc1ccc(F)c(CN2C(=O)C(C)Sc3cc(C(=O)NCc4c(F)cc(F)cc4F)ccc32)c1Cl ; CCCCCOc1ccc(F)c(CN2C(=O)C(C)Sc3cc(C(=O)NCc4c(F)cc(F)cc4F)ccc32)c1Cl ; CCCCCCOc1ccc(F)c(CN2C(=O)C(C)Sc3cc(C(=O)NCc4c(F)cc(F)cc4F)ccc32)c1Cl]

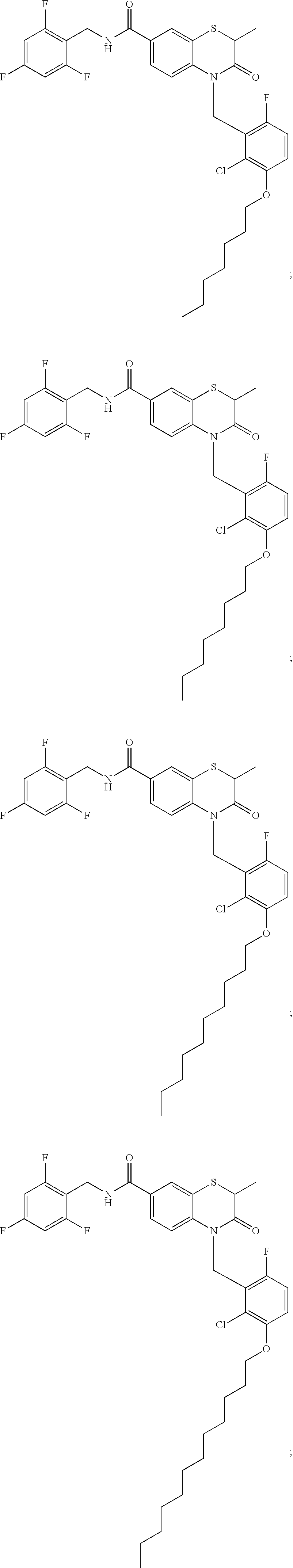 [SMILES: CCCCCCCOc1ccc(F)c(CN2C(=O)C(C)Sc3cc(C(=O)NCc4c(F)cc(F)cc4F)ccc32)c1Cl ; CCCCCCCCOc1ccc(F)c(CN2C(=O)C(C)Sc3cc(C(=O)NCc4c(F)cc(F)cc4F)ccc32)c1Cl ; CCCCCCCCCCOc1ccc(F)c(CN2C(=O)C(C)Sc3cc(C(=O)NCc4c(F)cc(F)cc4F)ccc32)c1Cl ; CCCCCCCCCCCCOc1ccc(F)c(CN2C(=O)C(C)Sc3cc(C(=O)NCc4c(F)cc(F)cc4F)ccc32)c1Cl]

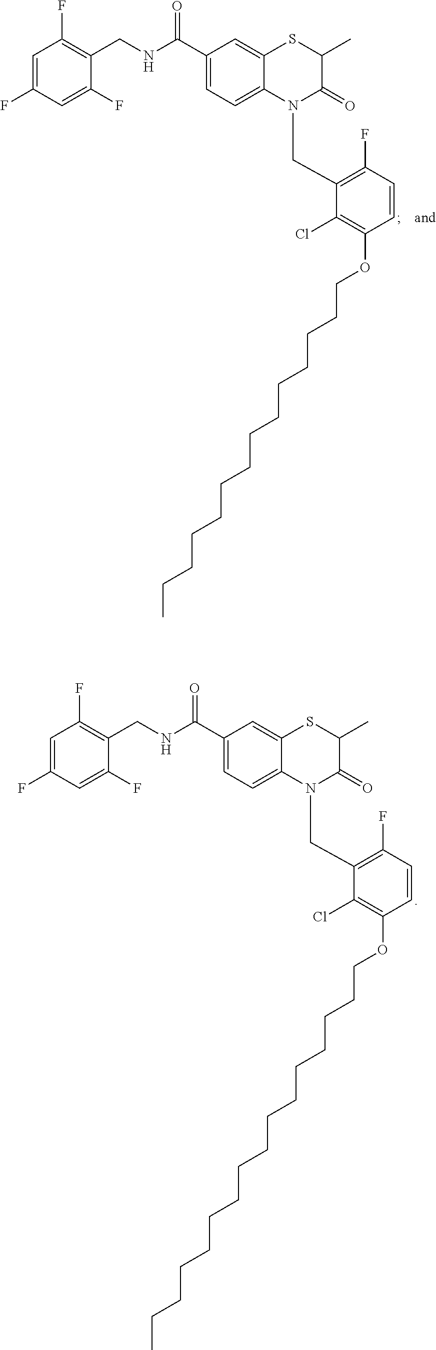 [SMILES: CCCCCCCCCCCCCCOc1ccc(F)c(CN2C(=O)C(C)Sc3cc(C(=O)NCc4c(F)cc(F)cc4F)ccc32)c1Cl ; CCCCCCCCCCCCCCCCOc1ccc(F)c(CN2C(=O)C(C)Sc3cc(C(=O)NCc4c(F)cc(F)cc4F)ccc32)c1Cl]

16-18. (canceled)

19. The compound of claim 6, wherein the compound of formula (Ia-iii) is selected from:

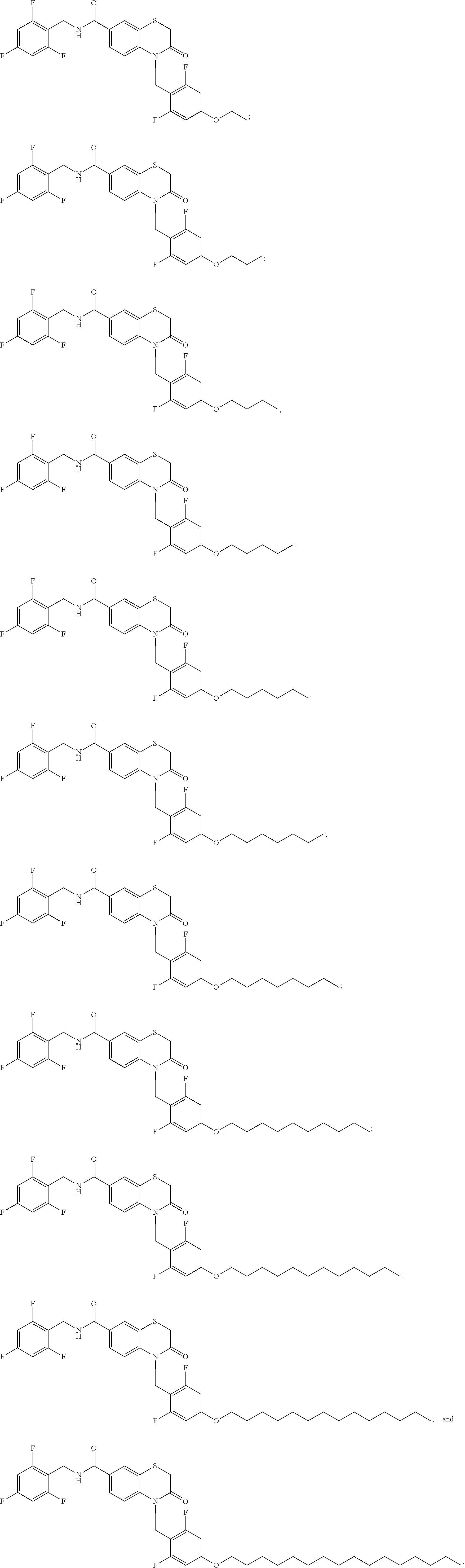

20. (canceled)

21. The compound of claim 8, wherein the compound of formula (Ia-iii) is selected from:

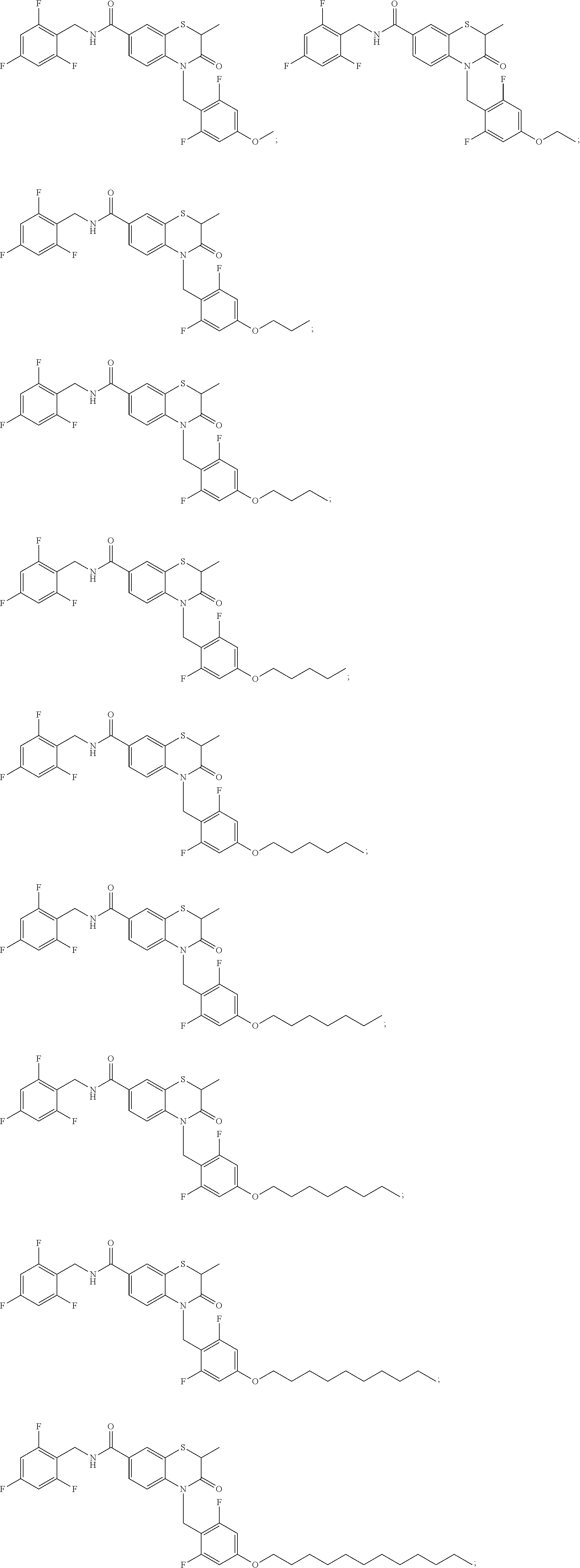

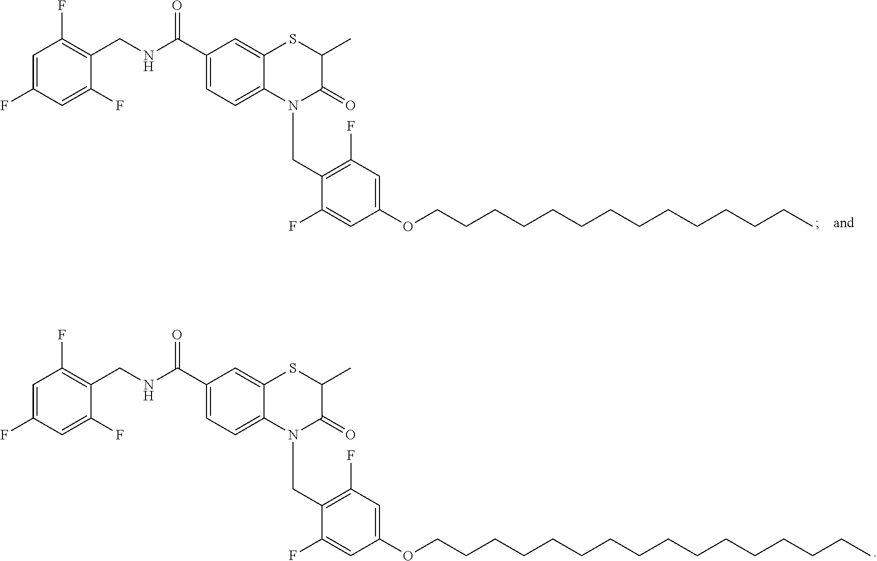 [SMILES: CCCCCCCCCCCCCCOc1cc(F)c(CN2C(=O)C(C)Sc3cc(C(=O)NCc4c(F)cc(F)cc4F)ccc32)c(F)c1 ; CCCCCCCCCCCCCCCCOc1cc(F)c(CN2C(=O)C(C)Sc3cc(C(=O)NCc4c(F)cc(F)cc4F)ccc32)c(F)c1]

22. The compound of claim 3, wherein R₂ is —NR₅R₆ or —NO₂.

23. The compound of claim 4, wherein R₂ is —NR₅R₆ or —NO₂.

24. The compound of claim 23, wherein the compound of formula (I-a) is selected from:

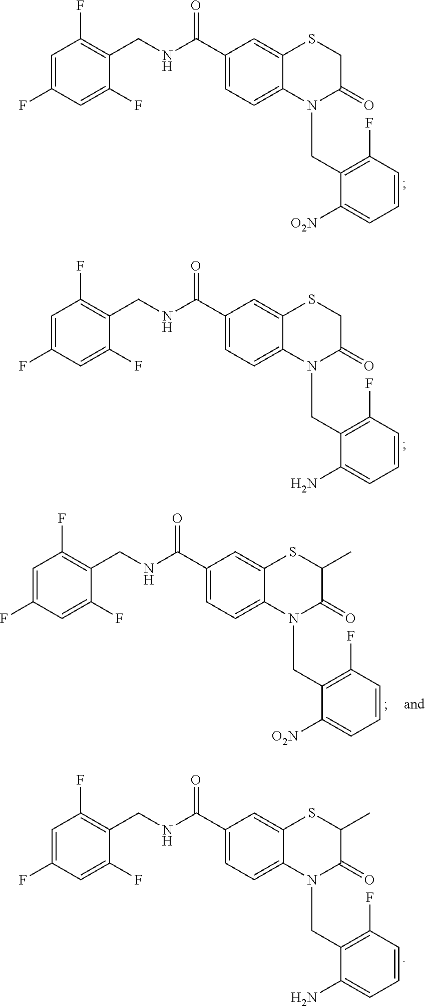 [SMILES: O=C(NCc1c(F)cc(F)cc1F)c1ccc2c(c1)SCC(=O)N2Cc1c(F)cccc1[N+](=O)[O-] ; Nc1cccc(F)c1CN1C(=O)CSc2cc(C(=O)NCc3c(F)cc(F)cc3F)ccc21 ; CC1Sc2cc(C(=O)NCc3c(F)cc(F)cc3F)ccc2N(Cc2c(F)cccc2[N+](=O)[O-])C1=O ; CC1Sc2cc(C(=O)NCc3c(F)cc(F)cc3F)ccc2N(Cc2c(N)cccc2F)C1=O]

25. (canceled)

26. The compound of claim 3, wherein the compound of formula (Ib) is a compound selected from a compound of formula (Ib-i), formula (Ib-ii) and formula (Ib-iii):

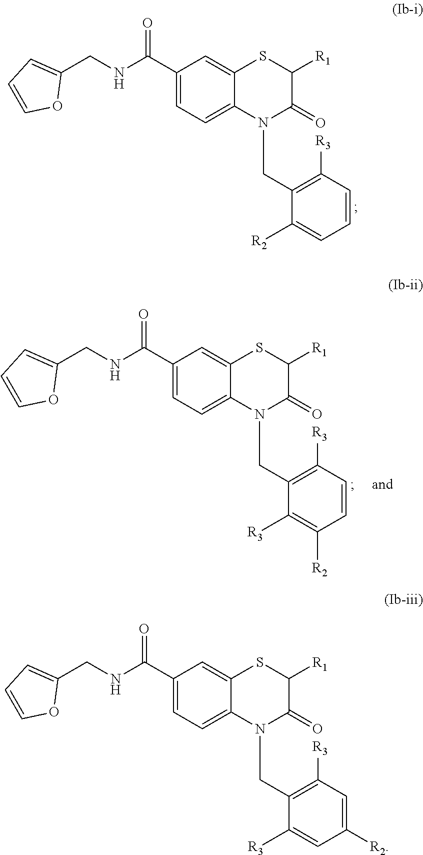

27. The compound of claim 26, wherein R₂ is —OR₄.

28. The compound of claim 27, wherein R₁ is H.

29. The compound of claim 28, wherein the compound of formula (Ib-i) is selected from:

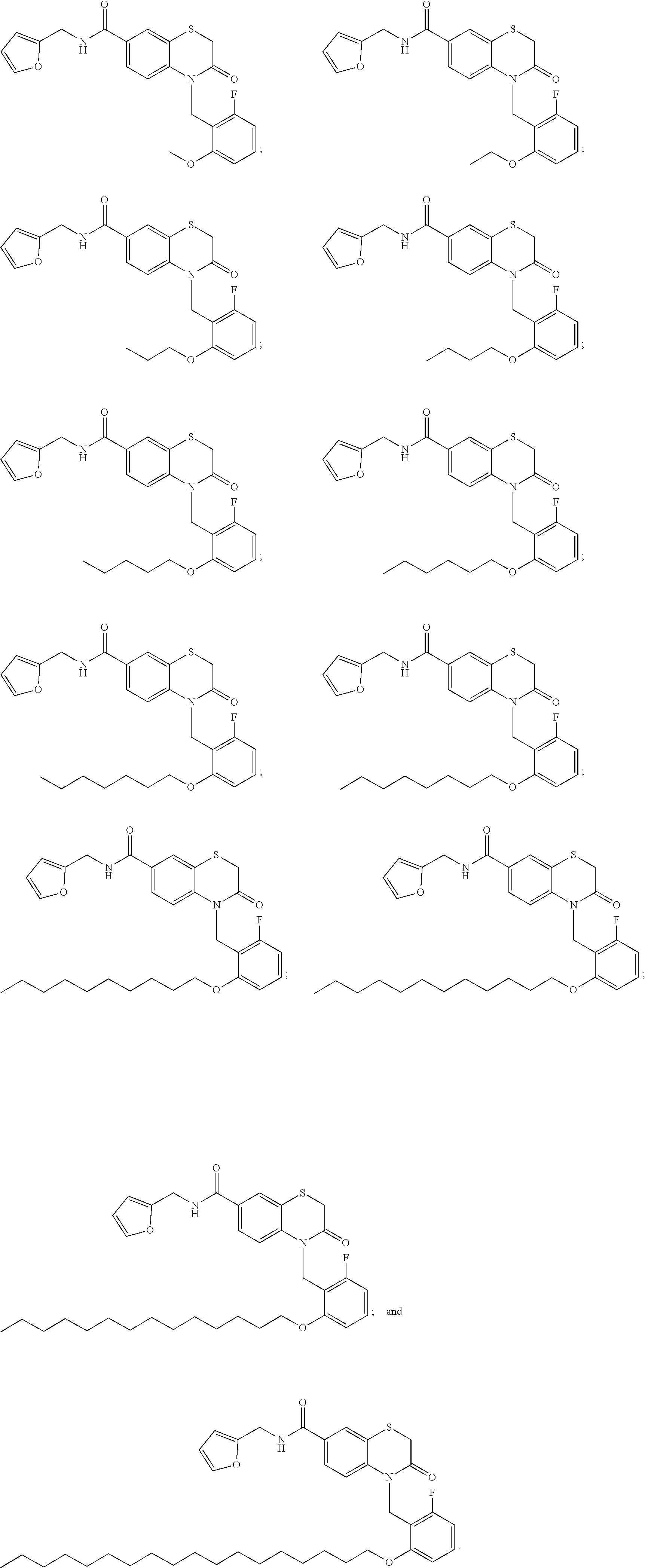

30. The compound of claim 27, wherein R₁ is C₁-C₄ alkyl.

31. The compound of claim 30, wherein the compound of formula (Ib-i) is selected from:

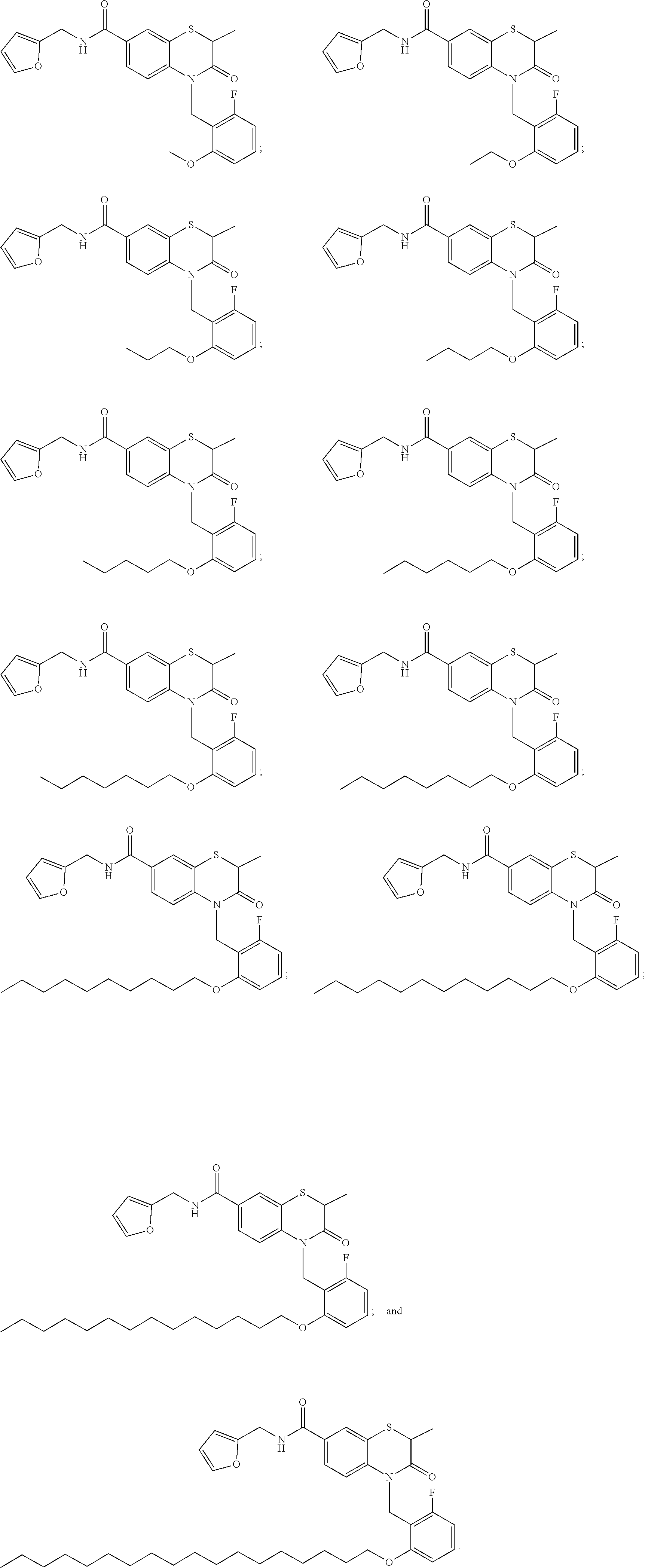

32-34. (canceled)

35. The compound of claim 28, wherein the compound of formula (Ib-ii) is selected from:

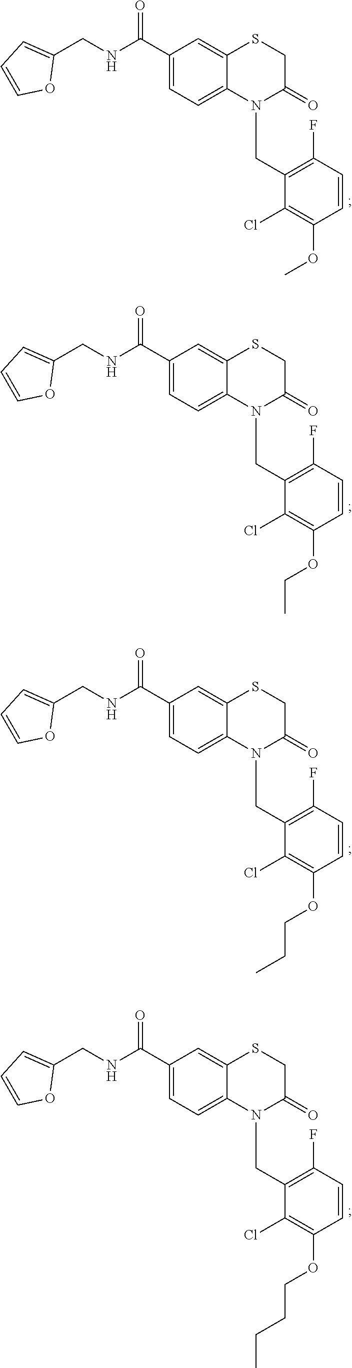 [SMILES: COc1ccc(F)c(CN2C(=O)CSc3cc(C(=O)NCc4ccco4)ccc32)c1Cl ; CCOc1ccc(F)c(CN2C(=O)CSc3cc(C(=O)NCc4ccco4)ccc32)c1Cl ; CCCOc1ccc(F)c(CN2C(=O)CSc3cc(C(=O)NCc4ccco4)ccc32)c1Cl ; CCCCOc1ccc(F)c(CN2C(=O)CSc3cc(C(=O)NCc4ccco4)ccc32)c1Cl]

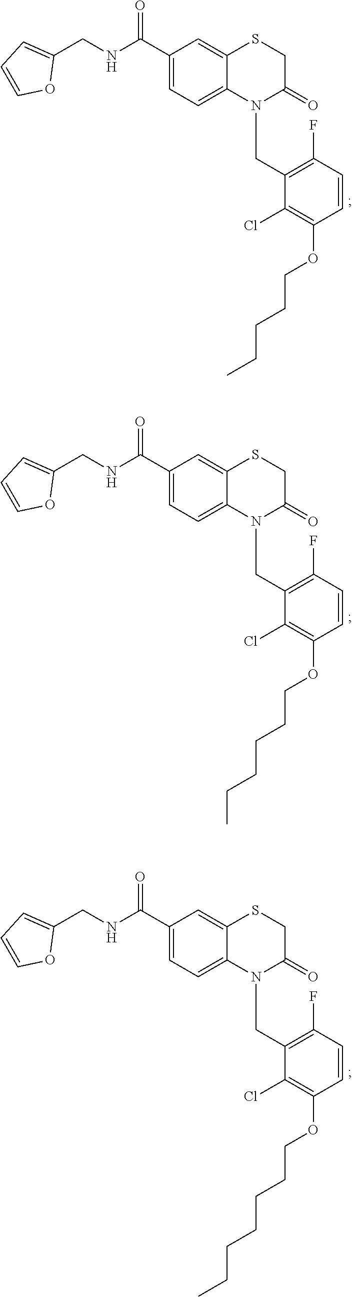 [SMILES: CCCCCOc1ccc(F)c(CN2C(=O)CSc3cc(C(=O)NCc4ccco4)ccc32)c1Cl ; CCCCCCOc1ccc(F)c(CN2C(=O)CSc3cc(C(=O)NCc4ccco4)ccc32)c1Cl ; CCCCCCCOc1ccc(F)c(CN2C(=O)CSc3cc(C(=O)NCc4ccco4)ccc32)c1Cl]

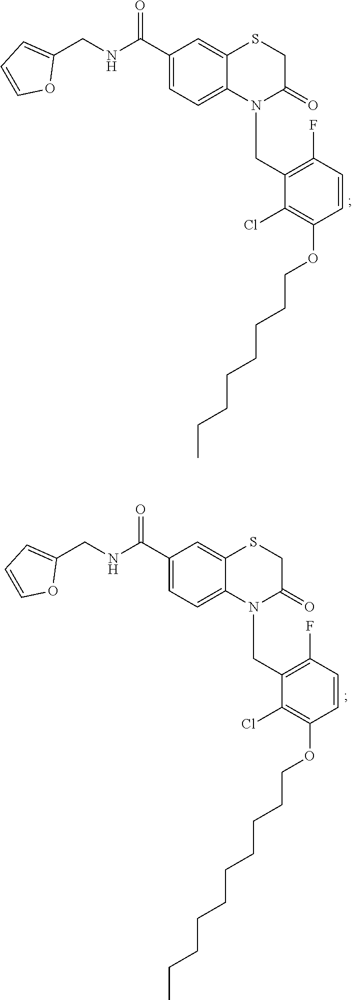 [SMILES: CCCCCCCCOc1ccc(F)c(CN2C(=O)CSc3cc(C(=O)NCc4ccco4)ccc32)c1Cl ; CCCCCCCCCCOc1ccc(F)c(CN2C(=O)CSc3cc(C(=O)NCc4ccco4)ccc32)c1Cl]

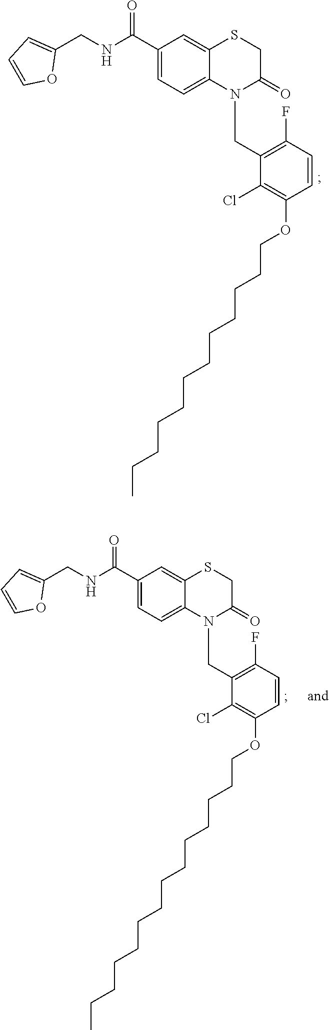 [SMILES: CCCCCCCCCCCCOc1ccc(F)c(CN2C(=O)CSc3cc(C(=O)NCc4ccco4)ccc32)c1Cl ; CCCCCCCCCCCCCCOc1ccc(F)c(CN2C(=O)CSc3cc(C(=O)NCc4ccco4)ccc32)c1Cl]

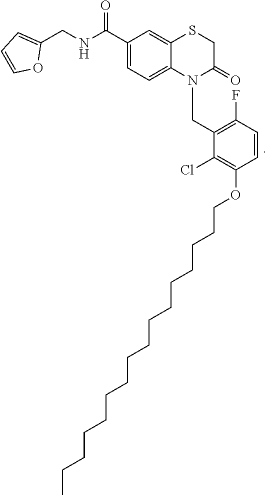 [SMILES: CCCCCCCCCCCCCCCCOc1ccc(F)c(CN2C(=O)CSc3cc(C(=O)NCc4ccco4)ccc32)c1Cl]

36. (canceled)

37. The compound of claim 30, wherein the compound of formula (Ib-ii) is selected from:

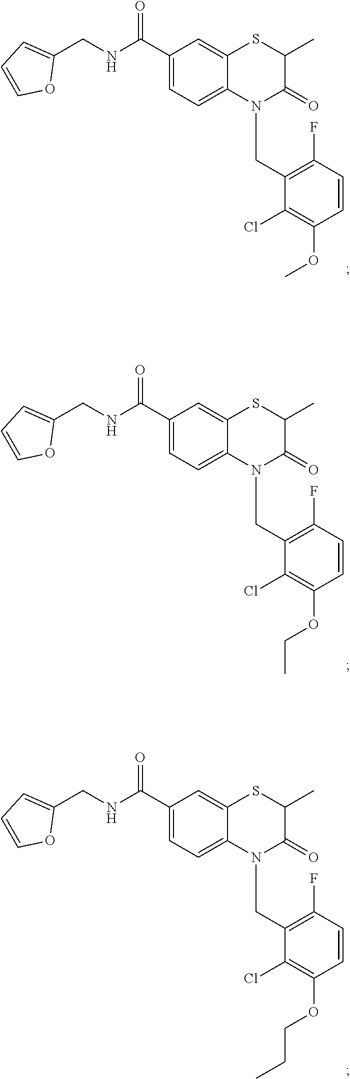 [SMILES: COc1ccc(F)c(CN2C(=O)C(C)Sc3cc(C(=O)NCc4ccco4)ccc32)c1Cl ; CCOc1ccc(F)c(CN2C(=O)C(C)Sc3cc(C(=O)NCc4ccco4)ccc32)c1Cl ; CCCOc1ccc(F)c(CN2C(=O)C(C)Sc3cc(C(=O)NCc4ccco4)ccc32)c1Cl]

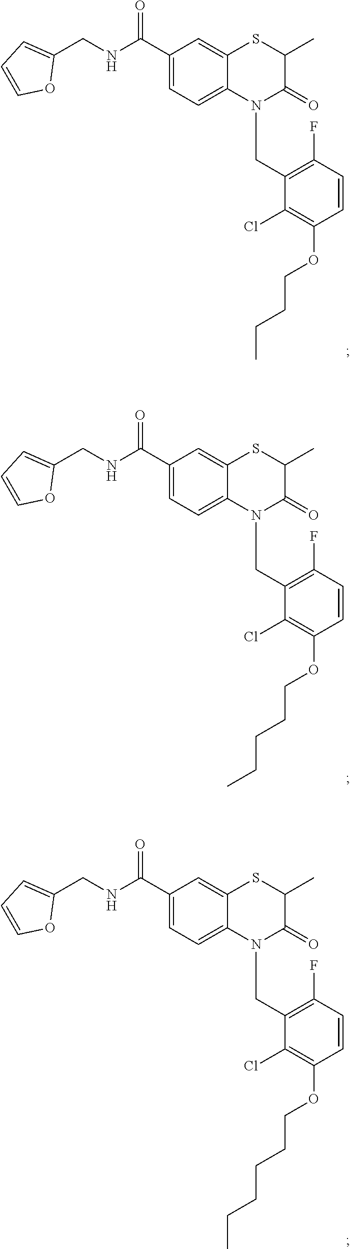 [SMILES: CCCCOc1ccc(F)c(CN2C(=O)C(C)Sc3cc(C(=O)NCc4ccco4)ccc32)c1Cl ; CCCCCOc1ccc(F)c(CN2C(=O)C(C)Sc3cc(C(=O)NCc4ccco4)ccc32)c1Cl ; CCCCCCOc1ccc(F)c(CN2C(=O)C(C)Sc3cc(C(=O)NCc4ccco4)ccc32)c1Cl]

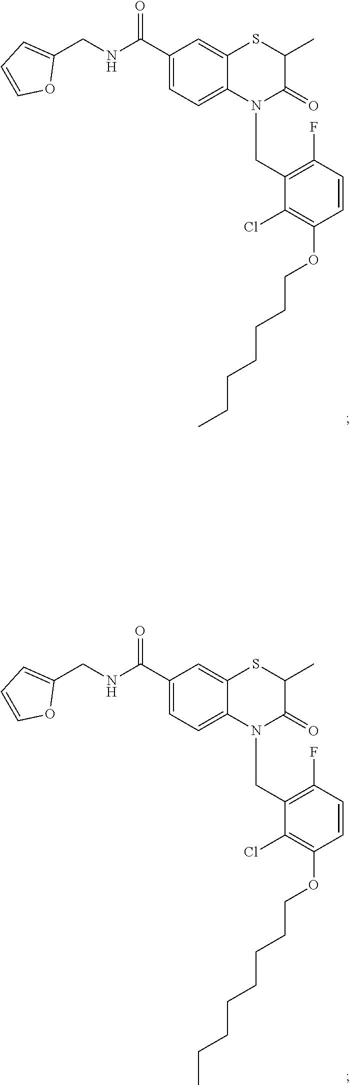 [SMILES: CCCCCCCOc1ccc(F)c(CN2C(=O)C(C)Sc3cc(C(=O)NCc4ccco4)ccc32)c1Cl ; CCCCCCCCOc1ccc(F)c(CN2C(=O)C(C)Sc3cc(C(=O)NCc4ccco4)ccc32)c1Cl]

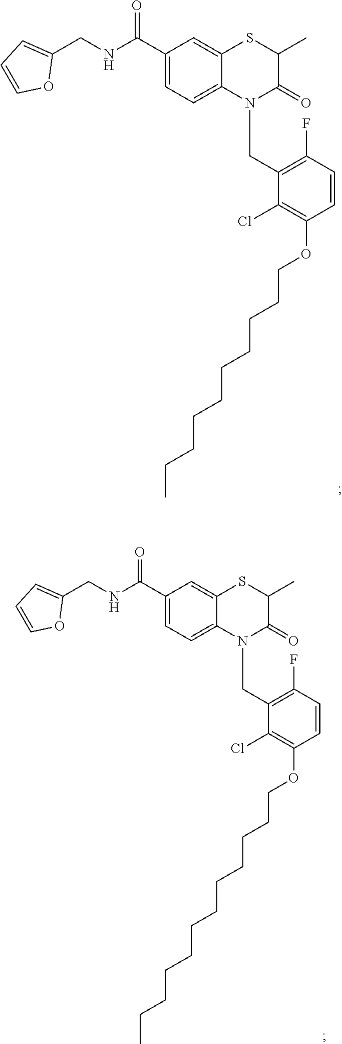 [SMILES: CCCCCCCCCCOc1ccc(F)c(CN2C(=O)C(C)Sc3cc(C(=O)NCc4ccco4)ccc32)c1Cl ; CCCCCCCCCCCCOc1ccc(F)c(CN2C(=O)C(C)Sc3cc(C(=O)NCc4ccco4)ccc32)c1Cl]

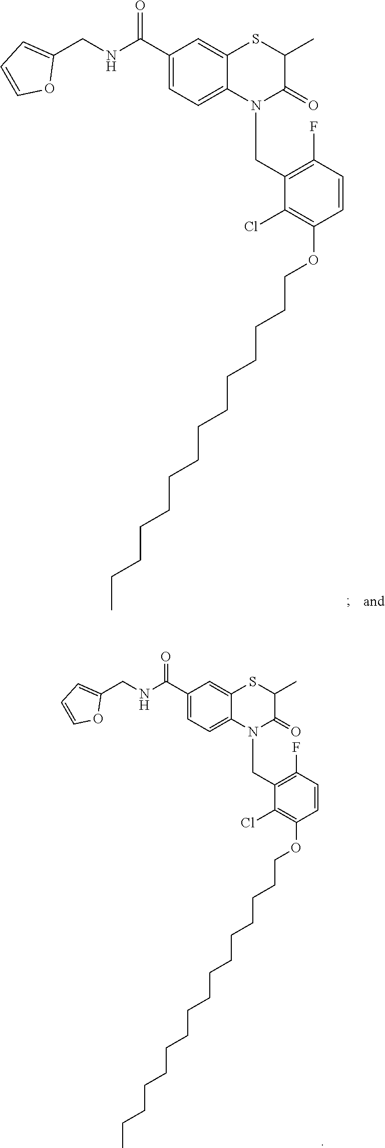 [SMILES: CCCCCCCCCCCCCCOc1ccc(F)c(CN2C(=O)C(C)Sc3cc(C(=O)NCc4ccco4)ccc32)c1Cl ; CCCCCCCCCCCCCCCCOc1ccc(F)c(CN2C(=O)C(C)Sc3cc(C(=O)NCc4ccco4)ccc32)c1Cl]

38-40. (canceled)

41. The compound of claim 28, wherein the compound of formula (Ib-iii) is selected from:

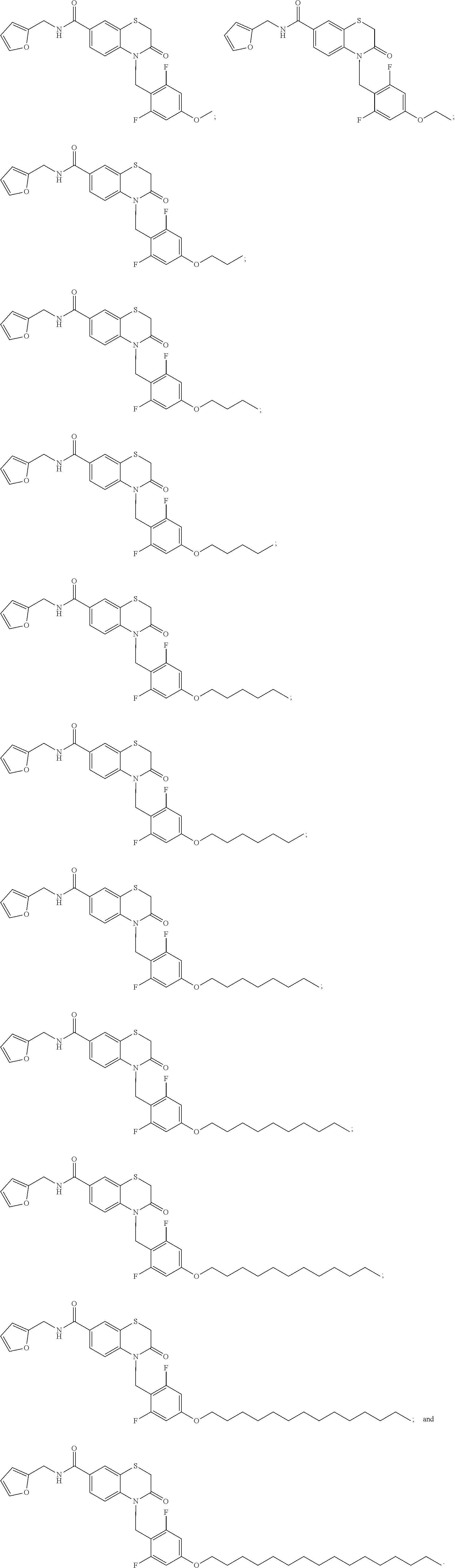

42. (canceled)

43. The compound of claim 30, wherein the compound of formula (Ib-iii) is selected from:

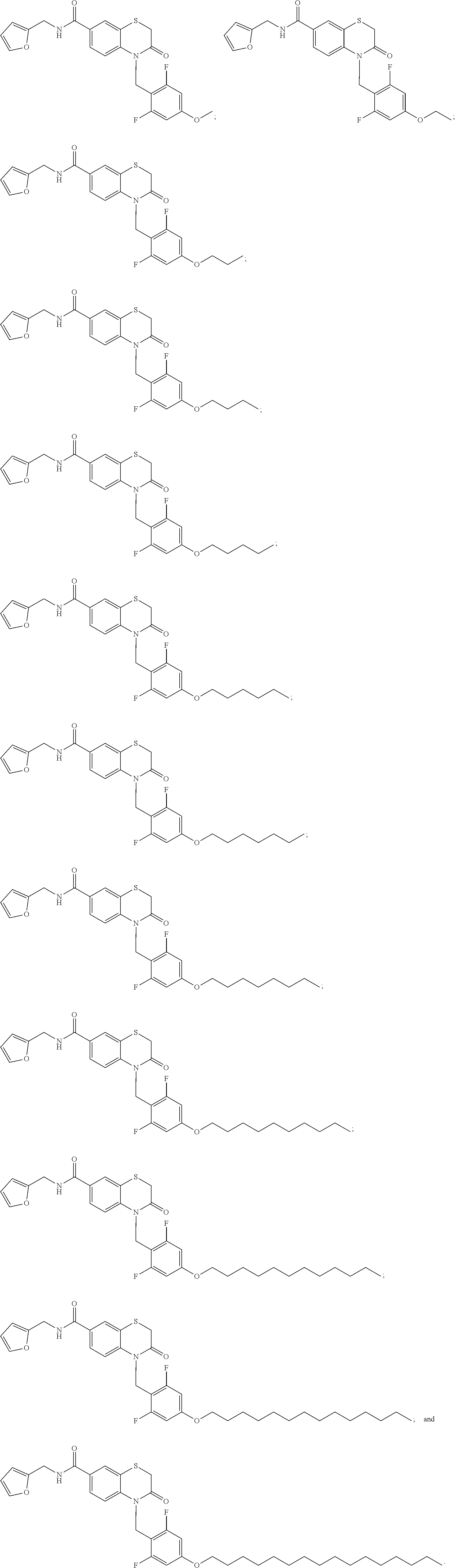

44. (canceled)

45. The compound of claim 26, wherein R₂ is —NR₅R₆ or —NO₂.

46. The compound of claim 45, wherein the compound of formula (I-b) is selected from:

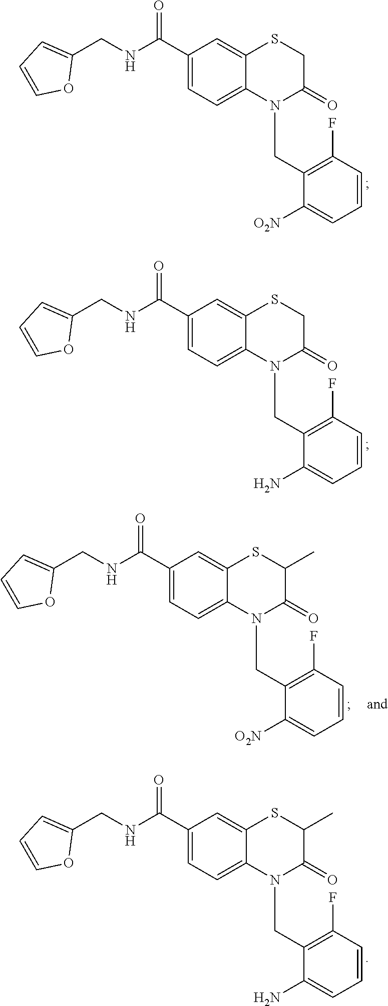 [SMILES: O=C(NCc1ccco1)c1ccc2c(c1)SCC(=O)N2Cc1c(F)cccc1[N+](=O)[O-] ; Nc1cccc(F)c1CN1C(=O)CSc2cc(C(=O)NCc3ccco3)ccc21 ; CC1Sc2cc(C(=O)NCc3ccco3)ccc2N(Cc2c(F)cccc2[N+](=O)[O-])C1=O ; CC1Sc2cc(C(=O)NCc3ccco3)ccc2N(Cc2c(N)cccc2F)C1=O]

47. A method for modulating Stimulator of Interferon Genes (STING), the method comprising contacting a cell with a compound of claim 1.

48. A method for activating or agonizing STING, the method comprising contacting a cell with a compound of claim 1.

49. A method for treating a disease, disorder, or condition associated with STING, the method comprising administering a therapeutically effective amount of a compound of claim 1 to a subject in need of treatment thereof.

50. The method of claim 49, wherein the disease, disorder, or condition is selected from cancer, a bacterial infection, a viral infection, a fungal infection, a parasitic infection, an immune-mediated disorder, a central nervous system disease, a peripheral nervous system disease, a neurodegenerative disease, a mood disorder, a sleep disorder, a cerebrovascular disease, a peripheral artery disease, and a cardiovascular disease.

51. (canceled)

52. The method of claim 50, wherein the cancer is selected from colorectal cancer, aero-digestive squamous cancer, lung cancer, brain cancer, liver cancer, stomach cancer, sarcoma, leukemia, lymphoma, multiple myeloma, ovarian cancer, uterine cancer, breast cancer, melanoma, prostate cancer, bladder cancer, pancreatic carcinoma, and renal carcinoma.

53. The method of claim 49, further comprising administering a second therapeutic agent.

54. The method of claim 53, wherein the second therapeutic agent is selected from an antiviral agent, an anti-inflammation agent, a chemotherapeutic agent, an anti-cancer vaccine, and hormonal therapy.

55. The method of claim 53, wherein the second therapeutic agent is selected from a B7 costimulatory molecule, interleukin-2, interferon-g, GM-CSF, a CTLA-4 antagonist, an IDO inhibitor or IDO/TDO inhibitor, a PD-1 inhibitor, a PD-L1 inhibitor, an OX-40 ligand, a LAG3 inhibitor, a CD40 ligand, a 41BB/CD137 ligand, a CD27 ligand, Bacille Calmette-Guerin (BCG), liposomes, alum, Freund's complete or incomplete adjuvant, a TLR agonist, and a detoxified endotoxin.

56. The method of claim 55, wherein the:
(a) CTLA-4 antagonist is ipilimumab or tremilimumab;
(b) IDO inhibitor or IDO/TDO inhibitor is epacadostat or GDC-0919;
(c) PD-1 inhibitor is selected from nivolumab, pembrolizumab, pidilizumab, AMP-224, and MDX-1106;
(d) PD-L1 inhibitor is selected from durvalumab, avelumab and atezolizumab; and
(e) TLR agonist is selected from Poly I: C, MPL, LPS, bacterial flagellin, imiquimod, resiquimod, loxoribine and a CpG dinucleotide.

57-60. (canceled)
